(19) United States
(12) Patent Application Publication
(10) Pub. No.: US 2026/0042756 A1
(43) Pub. Date: Feb. 12, 2026

(54) Organic Compound And Method For Synthesizing The Same

(71) Applicant: Semiconductor Energy Laboratory Co., Ltd., Kanagawa-ken (JP)

(72) Inventors: Yuko KUBOTA, Atsugi (JP); Kazuki KAJIYAMA, Hadano (JP); Yui YOSHIYASU, Atsugi (JP); Tomohiro KUBOTA, Atsugi (JP); Nobuharu OHSAWA, Zama, (JP); Toshiki SASAKI, Kawasaki (JP); Shinya FUKUZAKI, Atsugi (JP)

(73) Assignee: Semiconductor Energy Laboratory Co., Ltd., Kanagawa-ken (JP)

(21) Appl. No.: 19/289,950

(22) Filed: Aug. 4, 2025

(30) Foreign Application Priority Data
Aug. 9, 2024 (JP) 2024-134263

(51) Int. Cl. C07D 471/04

(52) U.S. Cl. CPC C07D 471/04

(57) ABSTRACT

A 1,10-phenanthroline derivative or a 2,2′-bipyridine derivative having different substituents at symmetrical positions is provided. An organic compound represented by General Formula (G1) or General Formula (G2) below is provided. In General Formula (G1) or (G2) below, any one of X² to X⁵ or any one of X⁶ to X⁹ represents a halogen or a trifluoromethanesulfonyl group, and the others represent hydrogens. Any one of R² to R⁵ or any one of R⁶ to R⁹ represents an aliphatic cyclic amino group. Note that a carbon to which the halogen or the trifluoromethanesulfonyl group is bonded and a carbon to which the aliphatic cyclic amino group is bonded are at line-symmetrical positions in a main skeleton (a 1,10-phenanthroline skeleton or a 2,2′-bipyridine skeleton).

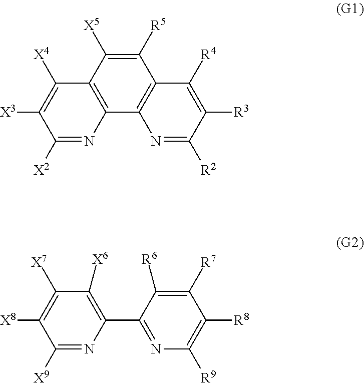

### BACKGROUND OF THE INVENTION

### 1. Field of the Invention

One embodiment of the present invention relates to an organic compound, an organic electronic device, a light-emitting device, an organic EL device, an electronic appliance, and a method for synthesizing an organic compound.

Note that one embodiment of the present invention is not limited to the above technical field. Examples of the technical field of one embodiment of the present invention include a compound, a light-emitting device, an organic EL device, a semiconductor device, a display device, a light-emitting apparatus, a power storage device, a memory device, an electronic appliance, a lighting device, an input device (e.g., a touch sensor), an input/output device (e.g., a touch panel), a method for driving any of them, and a method for manufacturing any of them.

### 2. Description of the Related Art

Recently, display devices have been expected to be applied to a variety of uses. Usage examples of large-sized display devices include a television device for home use (also referred to as TV or television receiver), digital signage, and a public information display (PID). In addition, a smartphone and a tablet terminal each including a touch panel, for example, are being developed as portable information terminals.

At the same time, an increase in the resolution of display devices is also required. Devices for virtual reality (VR), augmented reality (AR), substitutional reality (SR), or mixed reality (MR) are given as examples of devices requiring high-resolution display devices and have been actively developed.

Light-emitting apparatuses including light-emitting devices (also referred to as light-emitting elements) using organic compounds have been developed as display devices. Light-emitting devices utilizing electroluminescence (hereinafter referred to as EL; such devices are also referred to as organic EL devices or light-emitting devices) have features such as ease of reduction in thickness and weight, high-speed response to input signals, and driving with a constant voltage DC power source, and have been used in display devices.

Displays or lighting devices including light-emitting devices are suitable for a variety of electronic appliances, and research and development of materials and devices have progressed to obtain light-emitting devices with more favorable characteristics (see Patent Document 1, for example).

### REFERENCES

[Patent Document 1] Chinese Patent No. 111943949
[Non-Patent Document 1] Anton S. Abel et al., “1,10-Phenanthroline Carboxylic Acids for Preparation of Functionalized Metal-Organic Frameworks”, Asian Journal of Organic Chemistry, 2019, 8(11), pp. 2128-2142

### SUMMARY OF THE INVENTION

An object of one embodiment of the present invention is to provide a synthesis method for easily introducing different substituents to symmetrical positions in a 1,10-phenanthroline derivative or a 2,2′-bipyridine derivative.

An object of another embodiment of the present invention is to provide an organic compound in which different substituents are introduced to symmetrical positions in a 1,10-phenanthroline derivative or a 2,2′-bipyridine derivative. An object of another embodiment of the present invention is to provide an organic compound in which an aliphatic cyclic amino group is introduced to one of symmetrical positions in a 1,10-phenanthroline derivative or a 2,2′-bipyridine derivative and a substituent different from the aliphatic cyclic amino group (such a substituent is hereinafter also referred to as a substituent A) is introduced to the other of the symmetrical positions.

An object of another embodiment of the present invention is to provide a novel phenanthroline derivative or a novel bipyridine derivative. An object of another embodiment of the present invention is to provide a method for synthesizing a novel phenanthroline derivative or a novel bipyridine derivative.

Note that the description of these objects does not preclude the existence of other objects. One embodiment of the present invention does not necessarily achieve all of these objects. Other objects can be derived from the description of the specification, the drawings, and the claims.

One embodiment of the present invention is an organic compound represented by General Formula (G1) or General Formula (G2).

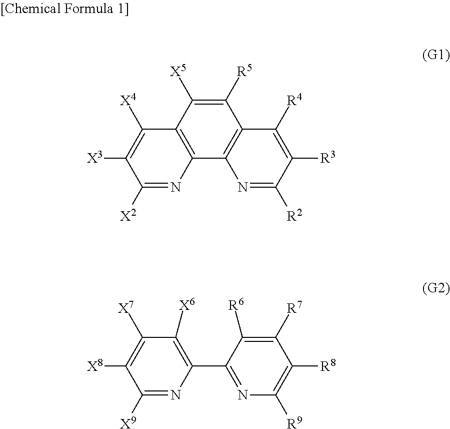

In General Formula (G1) or General Formula (G2) above, any one of X² to X⁵ or any one of X⁶ to X⁹ represents a halogen or a trifluoromethanesulfonyl group, and the others represent hydrogens. Any one of R² to R⁵ or any one of R⁶ to R⁹ represents an aliphatic cyclic amino group represented by General Formula (g1) below, and the others represent hydrogens. Note that a carbon to which the halogen or the trifluoromethanesulfonyl group is bonded and a carbon to which the group represented by General Formula (g1) below is bonded are at line-symmetrical positions in a main skeleton (a 1,10-phenanthroline skeleton or a 2,2′-bipyridine skeleton).

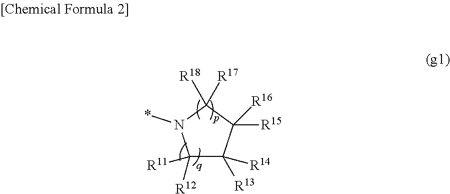

In General Formula (g1) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring.

Another embodiment of the present invention is an organic compound represented by any one of General Formulae (G1-1) to (G1-4) below.

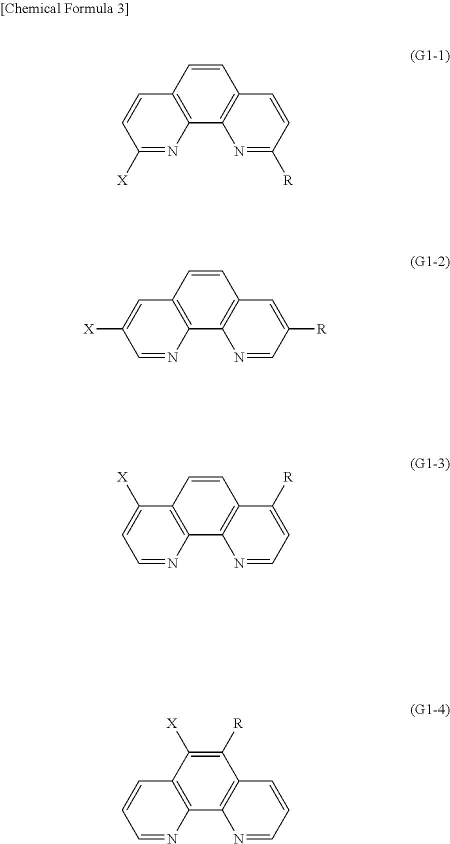

In General Formulae (G1-1) to (G1-4), X represents a halogen or a trifluoromethanesulfonyl group, and R represents an aliphatic cyclic amino group represented by General Formula (g1) below.

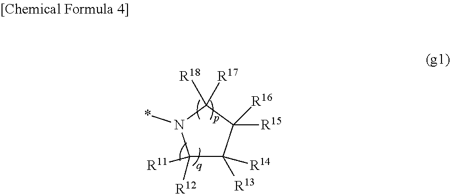

In General Formula (g1) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring.

Another embodiment of the present invention is an organic compound represented by any one of General Formulae (G2-1) to (G2-4) below.

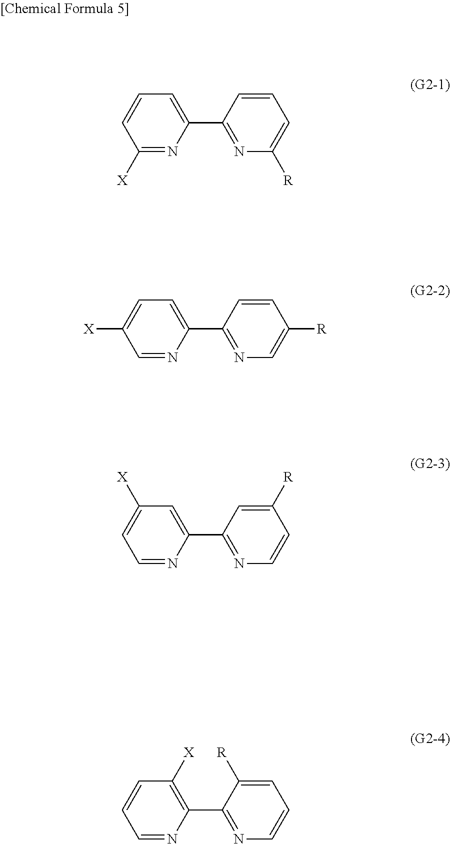

In General Formulae (G2-1) to (G2-4), X represents a halogen or a trifluoromethanesulfonyl group, and R represents an aliphatic cyclic amino group represented by General Formula (g1) below.

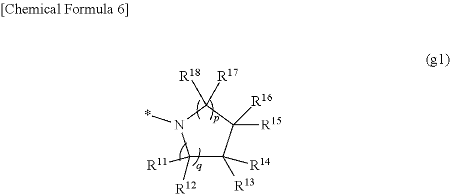

In General Formula (g1) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring.

Another embodiment of the present invention is an organic compound represented by General Formula (G3) below.

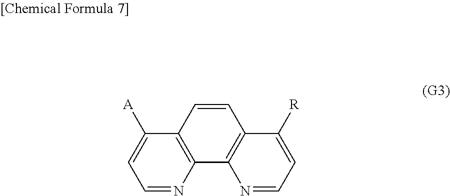

In General Formula (G3) above, R represents a group represented by General Formula (g1) below, and A represents a group represented by General Formula (g2) or General Formula (g3) below. In General Formula (G3), A and R are different substituents.

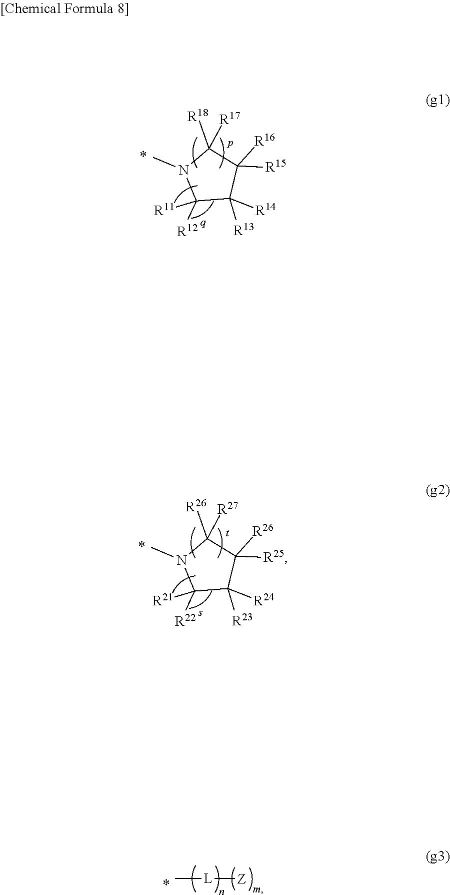

In General Formulae (g1) and (g2) above, R¹¹ to R¹⁸ and R²¹ to R²⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p, q, s, and t each independently represent 0 to 3. Any two of R¹¹ to R¹⁸ may be bonded to each other to form a ring, and any two of R²¹ to R²⁸ may be bonded to each other to form a ring. An aliphatic cyclic amino group represented by General Formula (g2) above may be condensed with an aromatic ring having 6 to 10 carbon atoms. In General Formula (g3) above, Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, a substituted or unsubstituted secondary amino group having 2 to 10 carbon atoms, a cyano group, a halogen, a hydroxy group, an amide group, and a carbonyl group, and m represents an integer of 1 to 3. When m is 2 or more, a plurality of Zs may be the same or different groups. L represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ls may be the same or different groups.

Another embodiment of the present invention is an organic compound represented by General Formula (G3-1) below.

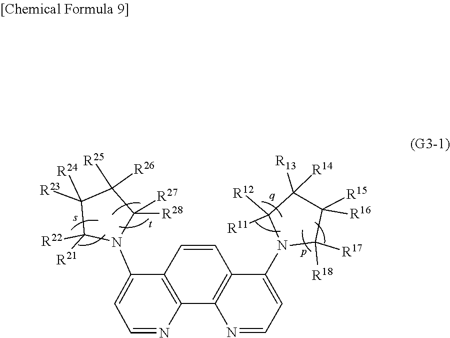

In General Formula (G3-1) above, R¹¹ to R¹⁸ and R²¹ to R²⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p, q, s, and t each independently represent 0 to 3. Any two of R¹¹ to R¹⁸ may be bonded to each other to form a ring, and any two of R²¹ to R²⁸ may be bonded to each other to form a ring. One aliphatic cyclic amino group in General Formula (G3-1) may be condensed with an aromatic ring having 6 to 10 carbon atoms. In General Formula (G3-1), different substituents are bonded to the 4- and 7-positions in a 1,10-phenanthroline skeleton.

Another embodiment of the present invention is an organic compound represented by General Formula (G3-2).

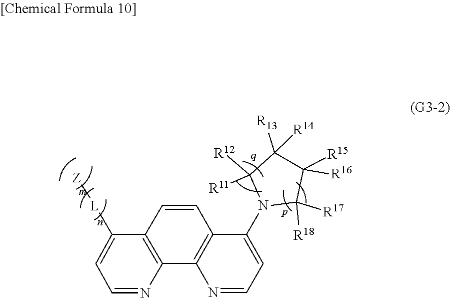

In General Formula (G3-2) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring. Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, a substituted or unsubstituted secondary amino group having 2 to 10 carbon atoms, a cyano group, a halogen, a hydroxy group, an amide group, and a carbonyl group, and m represents an integer of 1 to 3. When m is 2 or more, a plurality of Zs may be the same or different groups. L represents any one of a substituted or unsubstituted alkylene group having 1 to 10 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ls may be the same or different groups.

Another embodiment of the present invention is a method for synthesizing a 1,10-phenanthroline derivative or a 2,2′-bipyridine derivative having two different substituents bonded to respective carbons at symmetrical positions. The method includes the step of reacting, using an inorganic base and a solvent, an aliphatic cyclic amine with a 1,10-phenanthroline derivative or a 2,2′-bipyridine derivative having two halogens or trifluoromethanesulfonyl groups bonded to respective carbons at symmetrical positions.

Another embodiment of the present invention is a method for synthesizing a 1,10-phenanthroline derivative or a 2,2′-bipyridine derivative having two different substituents bonded to respective carbons at symmetrical positions. The method includes the step of heating and reacting, using potassium carbonate or potassium acetate and a solvent, an aliphatic cyclic amine with a 1,10-phenanthroline derivative or a 2,2′-bipyridine derivative having two halogens or triflate groups bonded to respective carbons at symmetrical positions.

Another embodiment of the present invention is a method for synthesizing a 1,10-phenanthroline derivative or a 2,2′-bipyridine derivative having two different substituents bonded to respective carbons at symmetrical positions. The method includes: a first step of reacting, using potassium carbonate or potassium acetate and a solvent, an aliphatic cyclic amine with a 1,10-phenanthroline derivative or a 2,2′-bipyridine derivative having two halogens or triflate groups bonded to respective carbons at symmetrical positions to obtain a 1,10-phenanthroline derivative or a 2,2′-bipyridine derivative having the aliphatic cyclic amine bonded to one of the carbons at the symmetrical positions and having the halogen or triflate group bonded to the other of the carbons at the symmetrical positions; and a second step of introducing another substituent to the other of the carbons at the symmetrical positions.

Another embodiment of the present invention is the above method for synthesizing a 1,10-phenanthroline derivative or a 2,2′-bipyridine derivative having two different substituents bonded to respective carbons at symmetrical positions, in which N-methyl-2-pyrrolidone is used as the solvent.

Another embodiment of the present invention is an organic semiconductor device including any of the organic compounds described above.

Another embodiment of the present invention is a light-emitting device including any of the organic compounds described above.

Another embodiment of the present invention is a light-receiving device including any of the organic compounds described above.

Another embodiment of the present invention is an organic electronic device using any of the organic compounds described above for a cap layer.

Another embodiment of the present invention is an electronic appliance including the above organic electronic device.

One embodiment of the present invention can provide a synthesis method for easily introducing different substituents to symmetrical positions in a 1,10-phenanthroline derivative or a 2,2′-bipyridine derivative.

Another embodiment of the present invention can provide an organic compound in which different substituents are introduced to two carbons at line-symmetrical positions in a 1,10-phenanthroline derivative or a 2,2′-bipyridine derivative. Another embodiment of the present invention can provide an organic compound in which an aliphatic cyclic amino group is introduced to one of two carbons at line-symmetrical positions in a 1,10-phenanthroline derivative or a 2,2′-bipyridine derivative and a substituent different from the aliphatic cyclic amino group (such a substituent is hereinafter also referred to as a substituent A) is introduced to the other carbon.

Another embodiment of the present invention can provide a novel phenanthroline derivative or a novel bipyridine derivative. Another embodiment of the present invention can provide a method for synthesizing a novel phenanthroline derivative or a novel bipyridine derivative.

One embodiment of the present invention can provide a novel light-emitting device, a novel display device, a novel display module, and a novel electronic appliance.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1C illustrate light-emitting devices.

FIGS. 2A and 2B are a top view and a cross-sectional view illustrating a light-emitting apparatus.

FIGS. 3A and 3B are perspective views illustrating a structure example of a display module.

FIGS. 4A and 4B are cross-sectional views illustrating structure examples of display devices.

FIG. 5 is a perspective view illustrating a structure example of a display device.

FIG. 6 is a cross-sectional view illustrating a structure example of a display device.

FIG. 7 is a cross-sectional view illustrating a structure example of a display device.

FIG. 8 is a cross-sectional view illustrating a structure example of a display device.

FIGS. 9A to 9D illustrate examples of electronic appliances.

FIGS. 10A to 10F illustrate examples of electronic appliances.

FIGS. 11A to 11G illustrate examples of electronic appliances.

FIGS. 12A to 12C show ¹H NMR charts of 4Cl7HidPhen.

FIGS. 13A to 13C show ¹H NMR charts of Hid2Phen.

FIGS. 14A to 14C show ¹H NMR charts of 4Cl7PrdPhen.

FIGS. 15A to 15C show ¹H NMR charts of 4Br7HidPhen.

FIGS. 16A to 16C show ¹H NMR charts of 4Br7PrdPhen.

FIGS. 17A to 17C show ¹H NMR charts of 2Cl9HidPhen.

FIGS. 18A to 18C show ¹H NMR charts of 4Cl4′HidBpy.

FIGS. 19A to 19C show ¹H NMR charts of Hid-DPPrdPhen.

FIGS. 20A to 20C show ¹H NMR charts of Hid-αNPrdPhen.

FIGS. 21A to 21C show ¹H NMR charts of Hid-αNPPhen.

FIGS. 22A to 22C show ¹H NMR charts of Hid-ceHBazPhen.

FIG. 23 shows luminance-current density characteristics of light-emitting devices 1-1 and 1-2 and a comparative light-emitting device 1.

FIG. 24 shows current efficiency-luminance characteristics of the light-emitting devices 1-1 and 1-2 and the comparative light-emitting device 1.

FIG. 25 shows luminance-voltage characteristics of the light-emitting devices 1-1 and 1-2 and the comparative light-emitting device 1.

FIG. 26 shows current density-voltage characteristics of the light-emitting devices 1-1 and 1-2 and the comparative light-emitting device 1.

FIG. 27 shows electroluminescence spectra of the light-emitting devices 1-1 and 1-2 and the comparative light-emitting device 1.

FIG. 28 shows luminance-current density characteristics of light-emitting devices 2-1 and 2-2 and a comparative light-emitting device 2.

FIG. 29 shows current efficiency-luminance characteristics of the light-emitting devices 2-1 and 2-2 and the comparative light-emitting device 2.

FIG. 30 shows luminance-voltage characteristics of the light-emitting devices 2-1 and 2-2 and the comparative light-emitting device 2.

FIG. 31 shows current density-voltage characteristics of the light-emitting devices 2-1 and 2-2 and the comparative light-emitting device 2.

FIG. 32 shows electroluminescence spectra of the light-emitting devices 2-1 and 2-2 and the comparative light-emitting device 2.

FIGS. 33A to 33C show ¹H NMR charts of Hid-AcuPhen.

### DETAILED DESCRIPTION OF THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

Ordinal numbers such as “first” and “second” in this specification and the like are used in order to avoid confusion among components and do not denote the priority or the order such as the order of steps or the stacking order. A term without an ordinal number in this specification and the like might be provided with an ordinal number in a claim in order to avoid confusion among components. A term with an ordinal number in this specification and the like might be provided with a different ordinal number in a claim. A term with an ordinal number in this specification and the like might not be provided with an ordinal number in a claim.

Note that in this specification and the like, a photoluminescence (PL) spectrum refers to a spectrum obtained by measuring the intensity of light emission by scanning the wavelength of light emission while fixing an excitation wavelength of excitation light in a fluorometry. Such a spectrum is also referred to as an emission spectrum in some cases. Note that an emission spectrum may include a fluorescence component and a phosphorescence component. In this specification and the like, an emission spectrum including a fluorescence component is particularly referred to as a fluorescence spectrum, and an emission spectrum including a phosphorescence component is particularly referred to as a phosphorescence spectrum in some cases.

### Embodiment 1

A 1,10-phenanthroline derivative and a 2,2′-bipyridine derivative have favorable electron-transport properties and are thus widely used as materials for organic semiconductor devices such as organic EL devices (which can be rephrased as light-emitting devices in this specification). For example, bathophenanthroline (abbreviation: BPhen) and 2,9-di(naphthalen-2-yl)-4,7-diphenyl-1,10-phenanthroline (abbreviation: NBPhen), which are 1,10-phenanthroline derivatives, have high electron-transport properties and have been used for a long time.

A 1,10-phenanthroline derivative having electron-donating groups at the 4- and 7-positions, such as 4,7-di-1-pyrrolidinyl-1,10-phenanthroline (abbreviation: Pyrrd-Phen), can have an increased electron density at nitrogens at the 1- and 10-positions of 1,10-phenanthroline. Thus, a composite material formed by co-evaporation of such a 1,10-phenanthroline derivative and a metal or a metal compound can be suitably used for an electron-injection layer of a light-emitting device and an n-type layer in an intermediate layer of a tandem light-emitting device. It is particularly preferable to use the composite material for a light-emitting device whose EL layer is processed by a photolithography method, in which case an increase in driving voltage can be inhibited.

A conventional 1,10-phenanthroline derivative, particularly a 1,10-phenanthroline derivative having substituents at the 4- and 7-positions as described above or the like, is what is called a C₂-symmetric organic compound, in which a 1,10-phenanthroline skeleton as a main skeleton has a symmetric structure and the substituents at the 4- and 7-positions are identical with each other. In the case where substituents are introduced to two symmetrical carbons (e.g., carbons at the 2- and 9-positions, the 3- and 8-positions, the 4- and 7-positions, or the 5- and 6-positions) of a 1,10-phenanthroline derivative, the synthesis of introducing the substituents can be performed by a nucleophilic substitution reaction or a Buchwald-Hartwig reaction of a 1,10-phenanthroline derivative having the carbons bonded to halogens, triflate groups, or the like. Note that the 1,10-phenanthroline derivative having the carbons bonded to halogens, triflate groups, or the like are readily available.

However, it has been difficult to introduce different substituents to the 2- and 9-positions, the 3- and 8-positions, the 4- and 7-positions, or the 5- and 6-positions by such a method because the 2- and 9-positions, the 3- and 8-positions, the 4- and 7-positions, or the 5- and 6-positions are equivalent in terms of chemical reactivity. Similarly, it has been difficult to introduce different substituents to the symmetrical positions in 2,2′-bipyridine.

In the case of 1,10-phenanthroline, for example, the synthesis of introducing asymmetric substituents as described above may be achieved using the Skraup reaction or the like; however, this method is not practical because it requires a highly hazardous reagent such as concentrated sulfuric acid and has low selectivity of reaction. Even if the synthesis can be achieved, the feasibility of purification is uncertain because a plurality of analogs are generated at the same time; thus, such a method cannot be regarded as suitable for supplying an asymmetric 1,10-phenanthroline derivative.

Similarly, in the case of a 2,2′-bipyridine derivative, the synthesis may be achieved by a cross coupling reaction when two kinds of pyridine derivatives having different substituents at the 4-positions and substituents such as halogens or pinacol borons at the 2-positions are prepared in advance. However, a pyridine compound in which the 2-position of pyridine is borylated is generally known to have low stability and be difficult to prepare and store, and homocoupling occurs as a side reaction in a coupling reaction; thus, a mixture of at least three kinds of reaction products is obtained in principle. For this reason, in order to isolate a desired compound from three kinds of compounds having similar properties and purify the compound, a heavy load is imposed on the purification step. In addition, a cross-coupling reaction is costly in general because of the use of an expensive transition metal catalyst. Although there may an option to use a relatively inexpensive transition metal catalyst such as nickel, this option is unlikely to be used actively because nickel is carcinogenic and requires preparing a safe and healthy work environment in a research facility or a production site. Moreover, preparing an environment in which such materials can be used is costly. In other words, conventional techniques prior to the present invention require at least a long time and a lot of cost to obtain the above-described asymmetric 1,10-phenanthroline or 2,2′-bipyridine derivative, and it is uncertain whether the compound can be synthesized and provided by the conventional techniques.

In this regard, the present inventors have found that, when a 1,10-phenanthroline derivative or a 2,2′-bipyridine derivative having a symmetric structure including two carbons at symmetrical positions to which halogens or trifluoromethanesulfonyl groups are bonded is reacted with an aliphatic cyclic amine using an inorganic base and a solvent, a 1,10-phenanthroline derivative or a 2,2′-bipyridine derivative having an asymmetric structure with the aliphatic cyclic amine bonded to one of the carbons at the symmetrical positions can be obtained.

It can be said that this method of one embodiment of the present invention enables an asymmetric 1,10-phenanthroline or 2,2′-bipyridine derivative to be stably synthesized and provided using a 1,10-phenanthroline or 2,2′-bipyridine derivative having a symmetrical structure, which is commercially available at a relatively low cost.

When the other halogen or trifluoromethanesulfonyl group of the asymmetric 1,10-phenanthroline or 2,2′-bipyridine derivative obtained by the above reaction is substituted with a given substituent, a 1,10-phenanthroline or 2,2′-bipyridine derivative having different substituents at symmetrical positions can be obtained.

As the inorganic base in the above reaction, a salt containing an alkali metal or an alkaline earth metal element, a hydroxide or a hydride of a metal element, or the like can be used, and potassium carbonate or potassium acetate is preferable to achieve high yield. As the solvent, a highly polar solvent such as N-methyl-2-pyrrolidone or ethanol can be used, and N-methyl-2-pyrrolidone is particularly preferable to achieve high yield.

In the above reaction, even when two or more equivalents of the aliphatic cyclic amine are added to the other halogen or trifluoromethanesulfonyl group of the 1,10-phenanthroline or 2,2′-bipyridine derivative, a 1,10-phenanthroline or 2,2′-bipyridine derivative with the aliphatic cyclic amine bonded to one of the carbons at the symmetrical positions can be obtained in high yield. Thus, a stable supply system can be constructed with a wide margin for conditions such as the amounts of raw materials, the reaction temperature, or the reaction time also in the mass production process.

One important factor for selectivity in the reaction for obtaining the asymmetric 1,10-phenanthroline or 2,2′-bipyridine derivative is a combination of a solvent and a base. As disclosed by Non-Patent Document 1, it is known that a symmetric 1,10-disubstituted phenanthroline derivative can be selectively obtained when an amine that dissolves in an organic solvent is used as a base, which will be described in detail below. Thus, if the reaction system maintains strong basicity, it is difficult to obtain asymmetric 1,10-monosubstituted phenanthroline.

Meanwhile, in the synthesis method of one embodiment of the present invention, an inorganic base with low solubility in an organic solvent, such as potassium carbonate, is used. Thus, basicity in the reaction system can be maintained in an optimum pH range for obtaining the asymmetric 1,10-monosubstituted phenanthroline, and this contributes to the selectivity of reaction. Similarly, since an acetate with weak acidity also affects the selectivity, a salt of a polybasic acid such as a phosphate, which can easily control pH and solubility in a solvent, can also be used as a base in the synthesis method of one embodiment of the present invention, and an asymmetric 1,10-phenanthroline or 2,2′-bipyridine derivative can be selectively obtained using such a base.

By the above synthesis method, an organic compound represented by General Formula (G1) below can be obtained.

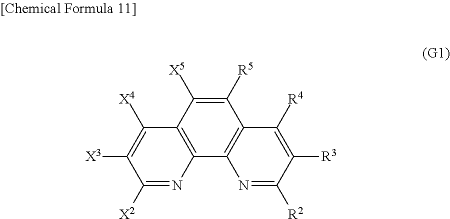

In General Formula (G1) above, any one of X² to X⁵ represents a halogen or a trifluoromethanesulfonyl group, and the others represent hydrogens. Any one of R² to R⁵ represents an aliphatic cyclic amino group represented by General Formula (g1) below, and the others represent hydrogens.

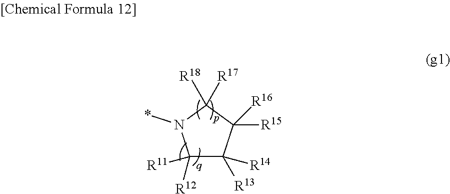

In General Formula (g1) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring.

In the organic compound represented by General Formula (G1) above, a carbon to which the halogen or the trifluoromethanesulfonyl group is bonded and a carbon to which the group represented by General Formula (g1) above is bonded are at symmetrical positions. In other words, in General Formula (G1) above, when X² represents a halogen or a trifluoromethanesulfonyl group, R² represents the aliphatic cyclic amino group represented by General Formula (g1) above. Similarly, when X³ is a halogen or a trifluoromethanesulfonyl group, R³ represents the aliphatic cyclic amino group represented by General Formula (g1) above; when X⁴ is a halogen or a trifluoromethanesulfonyl group, R⁴ represents the aliphatic cyclic amino group represented by General Formula (g1) above; and when X⁵ is a halogen or a trifluoromethanesulfonyl group, R⁵ represents the aliphatic cyclic amino group represented by General Formula (g1) above.

Specifically, one embodiment of the present invention is an organic compound represented by any one of General Formulae (G1-1) to (G1-4) below.

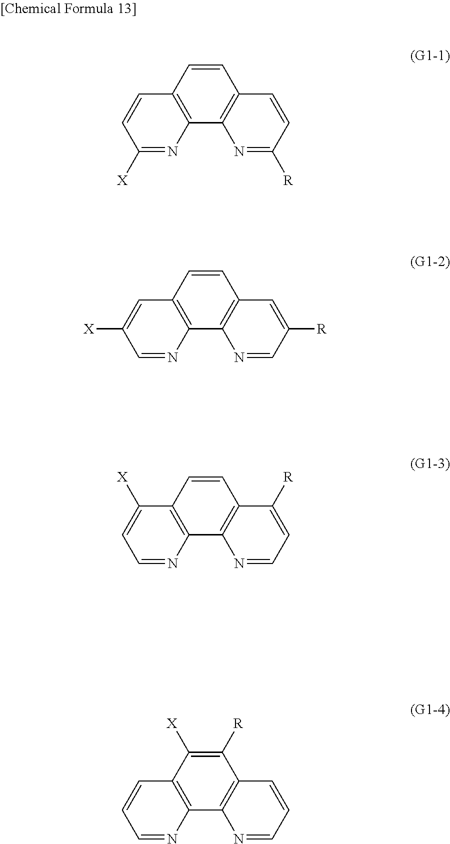

In General Formulae (G1-1) to (G1-4), X represents a halogen or a trifluoromethanesulfonyl group, and R represents the aliphatic cyclic amino group represented by General Formula (g1) above.

Similarly, by the above synthesis method, an organic compound represented by General Formula (G2) below can be obtained.

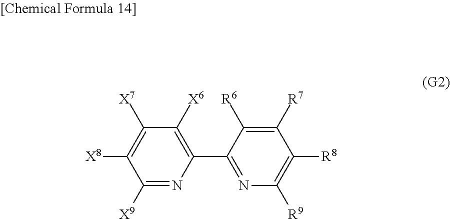

In General Formula (G2) above, any one of X⁶ to X⁹ represents a halogen or a trifluoromethanesulfonyl group, and the others represent hydrogens. Any one of R⁶ to R⁹ represents the aliphatic cyclic amino group represented by General Formula (g1) above, and the others represent hydrogens.

In the organic compound represented by General Formula (G2) above, a carbon to which the halogen or the trifluoromethanesulfonyl group is bonded and a carbon to which the group represented by General Formula (g1) above is bonded are at symmetrical positions. In other words, in General Formula (G2) above, when X⁶ represents a halogen or a trifluoromethanesulfonyl group, R⁶ represents the aliphatic cyclic amino group represented by General Formula (g1) above. Similarly, when X⁷ is a halogen or a trifluoromethanesulfonyl group, R⁷ represents the aliphatic cyclic amino group represented by General Formula (g1) above; when X⁸ is a halogen or a trifluoromethanesulfonyl group, R⁸ represents the aliphatic cyclic amino group represented by General Formula (g1) above; and when X⁹ is a halogen or a trifluoromethanesulfonyl group, R⁹ represents the aliphatic cyclic amino group represented by General Formula (g1) above.

Specifically, one embodiment of the present invention is an organic compound represented by any one of General Formulae (G2-1) to (G2-4) below.

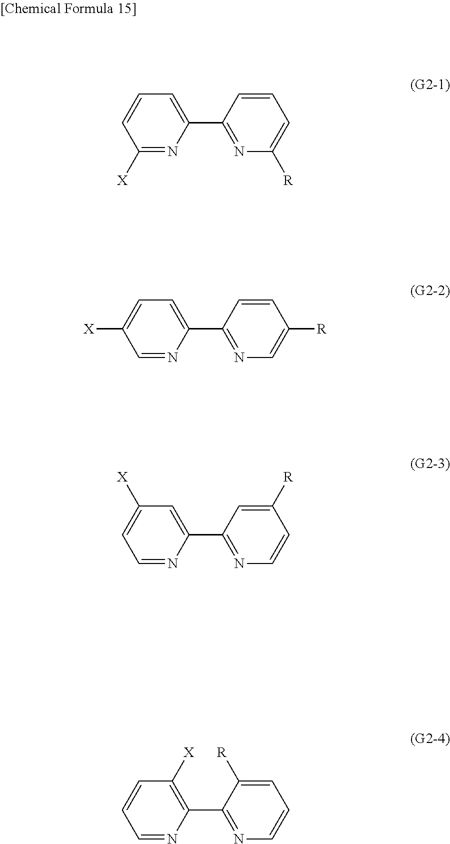

In General Formulae (G2-1) to (G2-4), X represents a halogen or a trifluoromethanesulfonyl group, and R represents the aliphatic cyclic amino group represented by General Formula (g1) above.

In the organic compound represented by any one of General Formula (G1), General Formula (G2), General Formulae (G1-1) to (G1-4), and General Formulae (G2-1) to (G2-4) above, any one of X² to X⁹ or X is a halogen or a trifluoromethanesulfonyl group. Thus, when a nucleophilic substitution reaction is performed using that organic compound, any of a variety of substituents can be introduced to the halogen or the trifluoromethanesulfonyl group. One example of an organic compound that can be synthesized using the organic compound of one embodiment of the present invention is an organic compound represented by General Formula (G3) below.

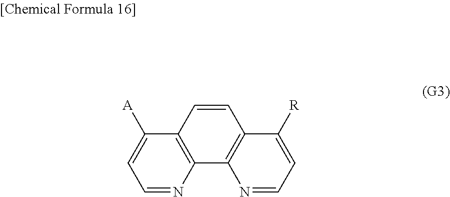

In General Formula (G3) above, R represents a group represented by General Formula (g1) below, and A represents a group represented by General Formula (g2) or General Formula (g3) below. In General Formula (G3), A and R are different substituents.

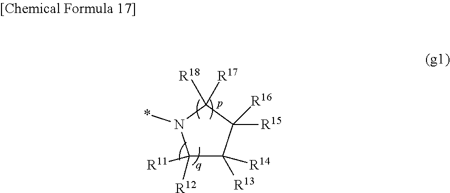

In General Formula (g1) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring.

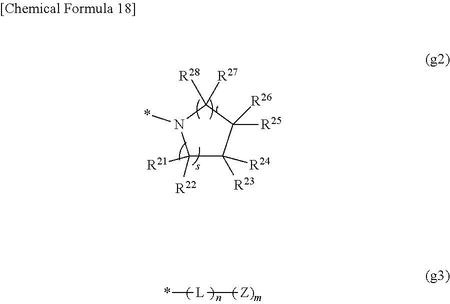

In General Formula (g2) above, R²¹ to R²⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and s and t each independently represent 0 to 3. Any two of R²¹ to R²⁸ may be bonded to each other to form a ring. An aliphatic cyclic amino group represented by General Formula (g2) above may be condensed with an aromatic ring having 6 to 10 carbon atoms. In General Formula (g3) above, Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, a substituted or unsubstituted secondary amino group having 2 to 10 carbon atoms, a cyano group, a halogen, a hydroxy group, an amide group, and a carbonyl group, and m represents an integer of 1 to 3. When m is 2 or more, a plurality of Zs may be the same or different groups. L represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ls may be the same or different groups.

In the organic compound represented by General Formula (G3) above, it is preferable that A be the organic compound represented by General Formula (g2) above, in which case electrons are donated to a 1,10-phenanthroline ring owing to the resonance effect and the electron density is increased in the 1,10-phenanthroline ring portion. Specifically, one embodiment of the present invention is an organic compound represented by General Formula (G3-1) below.

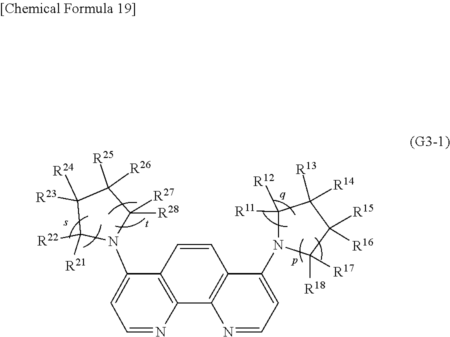

In General Formula (G3-1) above, R¹¹ to R¹⁸ and R²¹ to R²⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p, q, s, and t each independently represent 0 to 3. Any two of R¹¹ to R¹⁸ may be bonded to each other to form a ring, and any two of R²¹ to R²⁸ may be bonded to each other to form a ring. One aliphatic cyclic amino group in General Formula (G3-1) may be condensed with an aromatic ring having 6 to 10 carbon atoms. In General Formula (G3-1), different substituents are bonded to the 4- and 7-positions in a 1,10-phenanthroline skeleton.

In the organic compound represented by General Formula (G3) above, it is preferable that A be the organic compound represented by General Formula (g3) above, in which case 1,10-phenanthroline has different substituents as represented by (g1) and (g3) and thus parameters such as an electron-donating property, an affinity or exclusivity for a solvent including water, and heat resistance can be easily adjusted. Specifically, one embodiment of the present invention is preferably an organic compound represented by General Formula (G3-2) below.

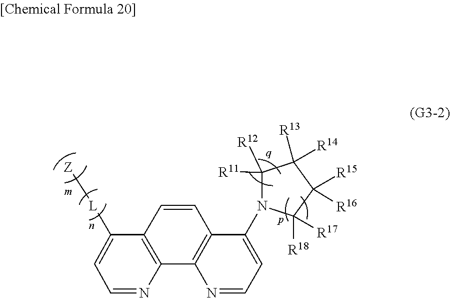

In General Formula (G3-2) above, R¹¹ to R¹⁸ each independently represent any one of a hydrogen (including a deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring. Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, and a substituted or unsubstituted secondary amino group having 2 to 10 carbon atoms, and m represents an integer of 1 to 3. When m is 2 or more, a plurality of Zs may be the same or different groups. L represents any one of a substituted or unsubstituted alkylene group having 1 to 10 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ls may be the same or different groups.

Preferable examples of the group represented by General Formula (g1) or General Formula (g2) above include groups represented by Structural Formulae (Am-1) to (Am-49) below.

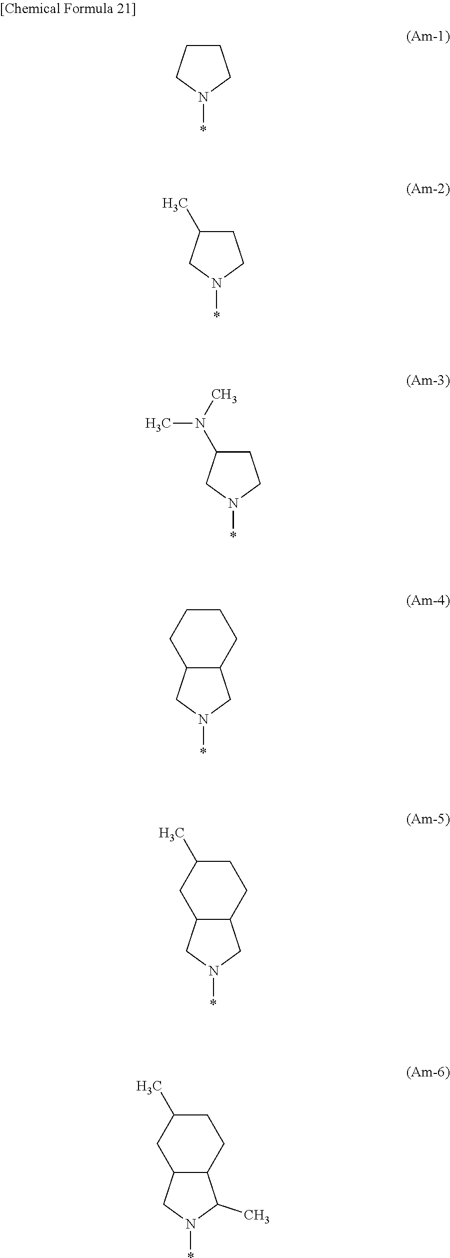

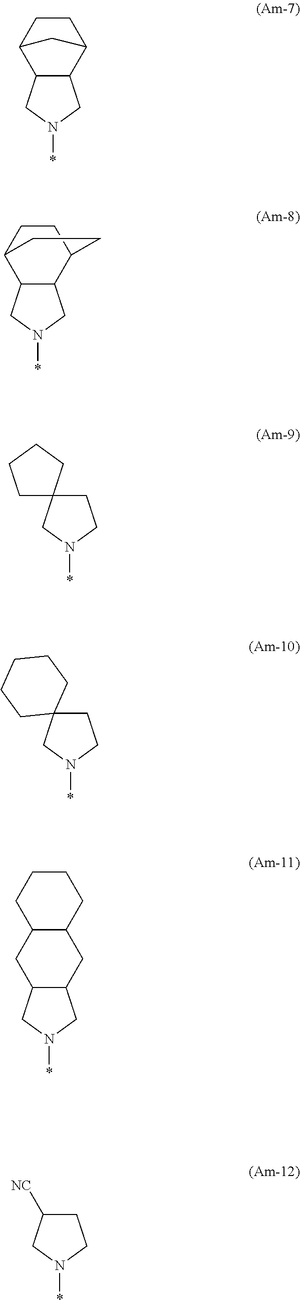

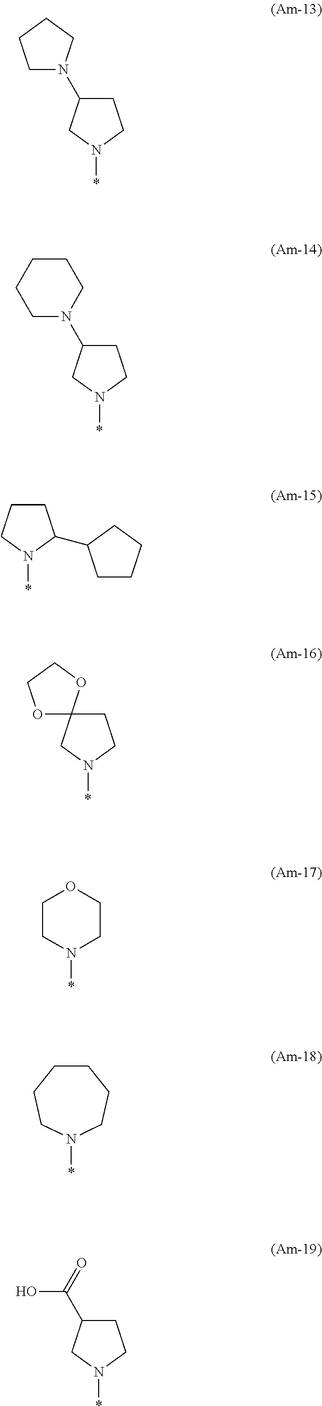

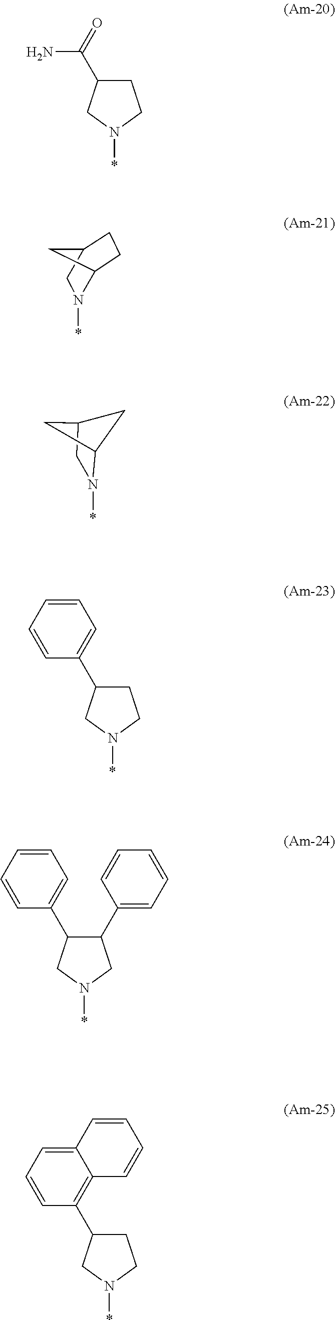

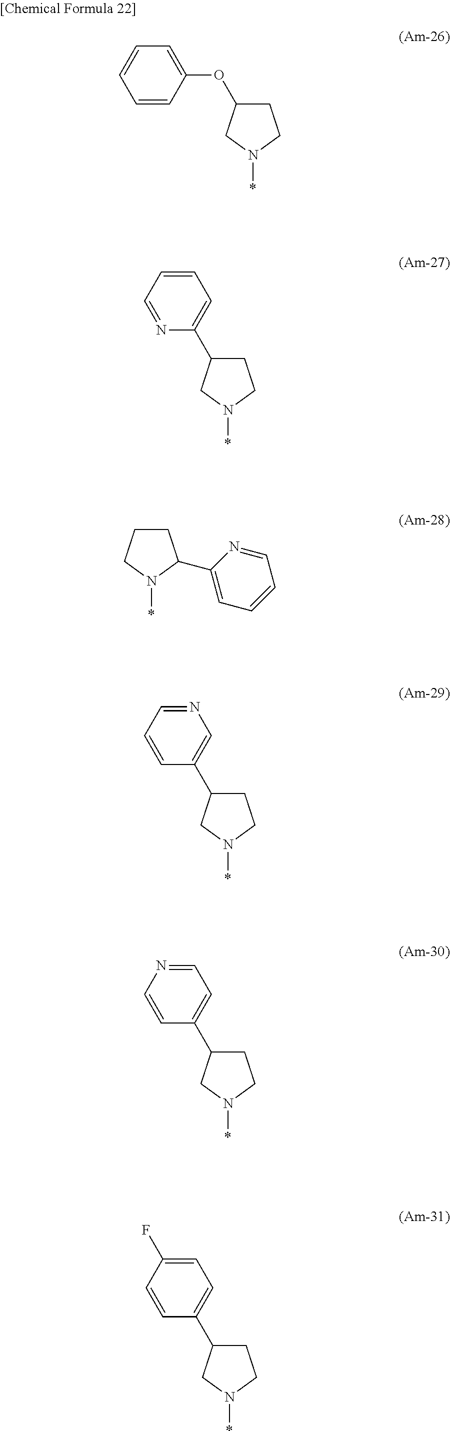

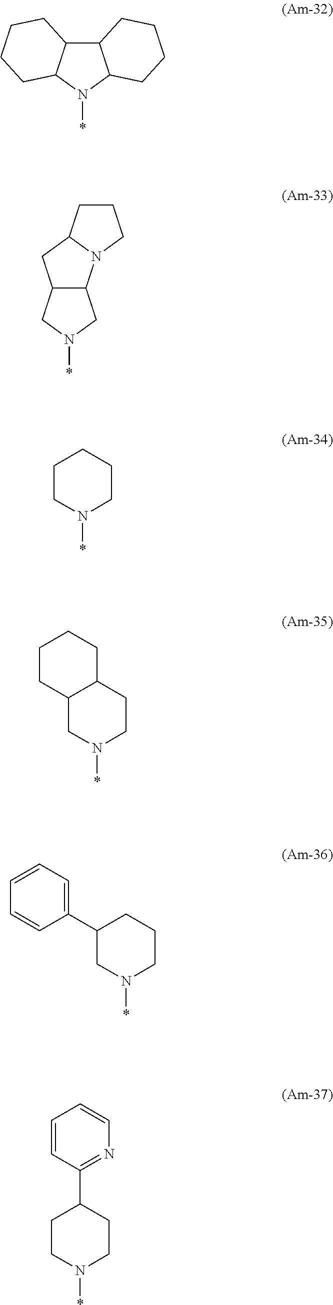

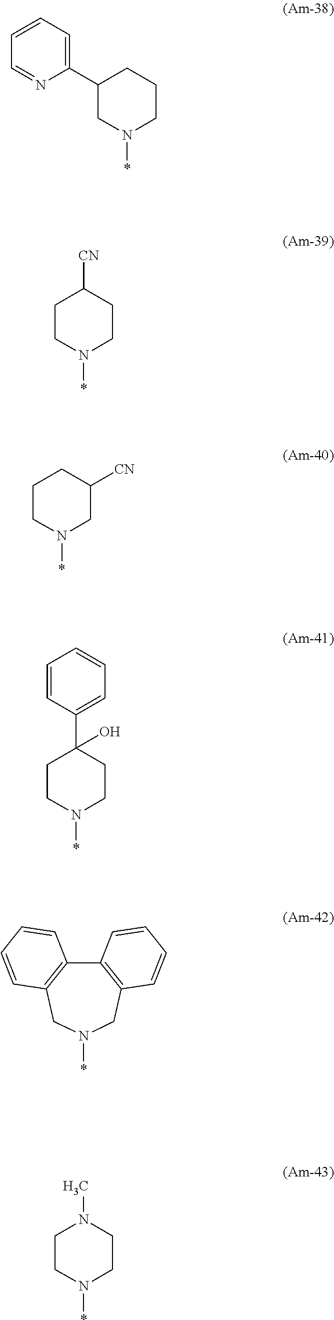

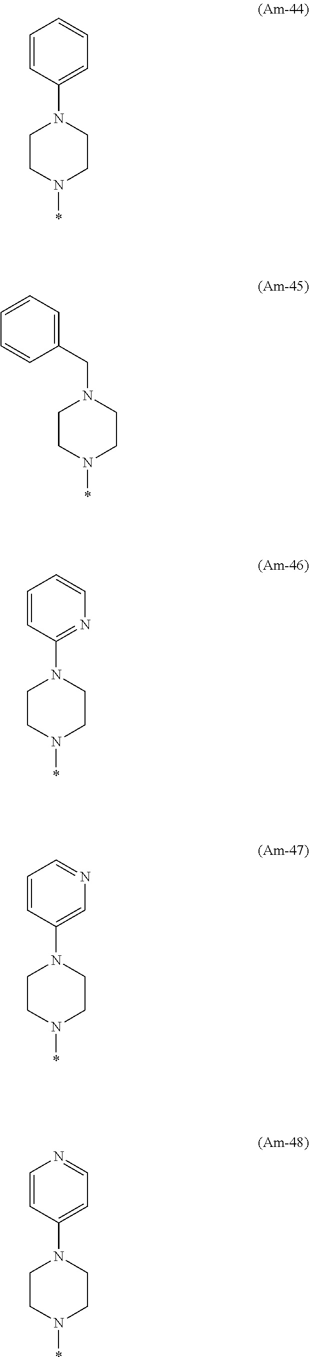

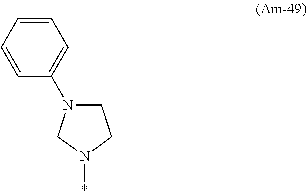

Examples of the alkyl group having 1 to 10 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a heptyl group, an octyl group, a 3-methylpentyl group, a 2-methylpentyl group, a 2-ethylbutyl group, a 1,2-dimethylbutyl group, a 2,3-dimethylbutyl group, a 2-ethylhexyl group, a 1-ethylpropyl group, a nonyl group, a 3,7-dimethyl-1-octyl group, a 3,7-dimethyl-2-octyl group, and a decyl group. Note that a tert-butyl group or a cyclohexyl group is particularly preferable because the refractive index can be reduced. In the case where the alkyl group having 1 to 10 carbon atoms has a substituent, the substituent can be a cycloalkyl group having 3 to 6 carbon atoms, an aryl group having 6 to 13 carbon atoms, a halogen, a cyano group, or the like.

Examples of the cycloalkyl group having 3 to 10 carbon atoms include a cyclopropyl group, a cyclobutyl group, a cyclopentyl group, a cyclohexyl group, a cycloheptyl group, a cyclooctyl group, a cyclononyl group, a cyclodecyl group, an adamantyl group, a bicyclo[2.2.1]heptyl group, a tricyclo[5.2.1.0(2,6)]decyl group, a noradamantyl group, a 1-methylcyclohexyl group, a bicyclo[2,2,2]octyl group, and a norbornyl group. In the case where the cycloalkyl group having 3 to 10 carbon atoms has a substituent, examples of the substituent include an alkyl group having 1 to 4 carbon atoms, a cycloalkyl group having 3 to 6 carbon atoms, an aryl group having 6 to 13 carbon atoms, a halogen, and a cyano group.

Examples of the alkoxyl group having 1 to 10 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neo-pentyloxy group, an n-hexyloxy group, an isohexyloxy group, a sec-hexyloxy group, a tert-hexyloxy group, a neo-hexyloxy group, and a cyclohexyloxy group.

Examples of the monovalent aromatic hydrocarbon group having 6 to 30 carbon atoms include a phenyl group, an o-tolyl group, a m-tolyl group, a p-tolyl group, a mesityl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthryl group, an anthryl group, a binaphthylphenyl group, a fluoranthenyl group, and a triphenylenyl group. In the case where the aryl group having 6 to 30 carbon atoms has a substituent, examples of the substituent include an alkyl group having 1 to 4 carbon atoms, a cycloalkyl group having 3 to 6 carbon atoms, an aryl group having 6 to 13 carbon atoms, a halogen, and a cyano group.

Specific examples of the heteroaryl group having 1 to 30 carbon atoms include a 1,3,5-triazin-2-yl group, a 1,2,4-triazin-3-yl group, a pyrimidin-4-yl group, a pyrazin-2-yl group, a 2-pyridyl group, a 3-pyridyl group, a 4-pyridyl group, a carbazolyl group, a dibenzofuranyl group, a dibenzothiophenyl group, a benzonaphthofuranyl group, a benzonaphthothiophenyl group, a dinaphthofuranyl group, a dinaphthothiophenyl group, an indolocarbazolyl group, a benzofurocarbazolyl group, a benzothienocarbazolyl group, an indenocarbazolyl group, a dibenzocarbazolyl group, an indolyl group, a pyrrolyl group, a 1,2,3-triazol-yl group, and a 1,2,4-triazol-yl group. In the case where the heteroaryl group having 1 to 30 carbon atoms has a substituent, examples of the substituent include an alkyl group having 1 to 4 carbon atoms, a cycloalkyl group having 3 to 6 carbon atoms, an aromatic hydrocarbon group having 6 to 13 carbon atoms, a halogen, and a cyano group.

Examples of the alkylene group having 1 to 3 carbon atoms include a methylene group, an ethylene group, and a propylene group. As the cycloalkylene group having 3 to 10 carbon atoms, a divalent group obtained by removing one hydrogen atom from the above-mentioned cycloalkyl group having 3 to 10 carbon atoms can be used, for example.

Examples of the divalent heterocyclic group having 1 to 25 carbon atoms include a pyrimidine-diyl group, a pyrazine-diyl group, a pyridazine-diyl group, a triazine-diyl group, a bipyridine-diyl group, a phenanthroline-diyl group, a quinoxaline-diyl group, a dibenzoquinoxaline-diyl group, a quinazoline-diyl group, a benzoquinazoline-diyl group, a dibenzoquinazoline-diyl group, an imidazole-diyl group, a triazole-diyl group, an oxadiazole-diyl group, a benzimidazole-diyl group, a furodiazine-diyl group, a benzofuropyrimidine-diyl group, a thiophene-diyl group, a furan-diyl group, a benzothiophene-diyl group, a benzofuran-diyl group, a dibenzothiophene-diyl group, a dibenzofuran-diyl group, a benzonaphthothiophene-diyl group, a benzonaphthofuran-diyl group, a dinaphthothiophene-diyl group, a dinaphthofuran-diyl group, a piperazine-diyl group, a hexahydropyrimidine-diyl group, a hexahydrotriazine-diyl group, a decahydroquinoxaline-diyl group, a decahydronaphthryridine-diyl group, an imidazolidine-diyl group, an octahydropyrrolopyridine-diyl group, and an octahydropyrrolopyrrole-diyl group. In the case where the divalent heterocyclic group having 1 to 25 carbon atoms has a substituent, examples of the substituent include an alkyl group having 1 to 4 carbon atoms, a cycloalkyl group having 3 to 6 carbon atoms, and an aryl group having 6 to 13 carbon atoms.

Examples of the divalent aromatic hydrocarbon group having 6 to 25 carbon atoms include a phenylene group, a biphenyl-diyl group, a naphthalene-diyl group, a fluorene-diyl group, an acenaphthene-diyl group, an anthracene-diyl group, a phenanthrene-diyl group, a terphenyl-diyl group, a triphenylene-diyl group, a tetracene-diyl group, a benzanthracene-diyl group, a pyrene-diyl group, and a spirobi[9H-fluorene]-diyl group. In the case where the arylene group having 6 to 30 carbon atoms has a substituent, examples of the substituent include an alkyl group having 1 to 4 carbon atoms, a cycloalkyl group having 3 to 6 carbon atoms, and an aryl group having 6 to 13 carbon atoms.

Note that the organic compound represented by General Formula (G3) preferably includes one or two alkyl groups, in which case the alkyl group(s) is less likely to hinder the electron-transport property. The alkyl group(s) can reduce the refractive index of a film including the organic compound, and a larger number of alkyl groups can further reduce the refractive index of an evaporated film and improve the emission efficiency of a light-emitting device. Meanwhile, it is known that the alkyl group(s) tends to reduce the electron-transport property. The organic compound of one embodiment of the present invention can be used to form a film with a low refractive index even when the organic compound includes a small number of hydrocarbon groups; thus, both the electron-transport property and the low refractive index of the film can be achieved.

When the organic compound represented by General Formula (G3) is an organic compound having at least one alkyl group, the alkyl group is preferably bonded to a phenyl group. That is, the organic compound represented by General Formula (G3) preferably has a phenyl group having an alkyl group. In the case where a phenyl group has two alkyl groups, the two alkyl groups are preferably at the 3- and 5-positions of the phenyl group at the end because of the easy availability of a material for synthesis. Note that the phenyl group having the alkyl group(s) is particularly preferably a 4-cyclohexylphenyl group, a 3′,5′-ditertiarybutylbiphenyl group, a 3′,5′-dicyclohexylbiphenyl group, or the like.

The secondary amino group having 2 to 10 carbon atoms is preferably a cyclic secondary amine, such as a pyrrolidin-1-yl group, an isoindol-2-yl group, a dihydroisoindol-2-yl group, a tetrahydroisoindol-2-yl group, a hexahydroisoindol-2-yl group, a hexahydroisoindolin-2-yl group, a piperidin-1-yl group, an aziridin-1-yl group, an azetidin-1-yl group, an octahydrocyclopenta[c]pyrrol-2-yl group, an octahydro-4,7-methano-1H-isoindol-2-yl group, a 2-azabicyclo[3.1.0]hexan-2-yl group, a 3-azabicyclo[3.1.0]hexan-2-yl group, a 3-azabicyclo[3.2.0]heptan-2-yl group, a 5-azaspiro[3.4]octan-5-yl group, an 8-azabicyclo[3.2.1]octan-8-yl group, a 7-azabicyclo[2.2.1]heptan-7-yl group, a 5-azaspiro[2.4]heptan-5-yl group, a 5-azabicyclo[2.1.1]hexan-5-yl group, a dimethylamino group, a diethylamino group, a diisopropylamino group, a diphenylamino group, or a dicyclohexylamino group. In the case where the cyclic secondary amino group having 2 to 10 carbon atoms has a substituent, examples of the substituent include an alkyl group having 1 to 4 carbon atoms, a cycloalkyl group having 3 to 6 carbon atoms, and an aryl group having 6 to 13 carbon atoms.

Examples of the organic compounds represented by General Formulae (G1) and (G2) above include organic compounds represented by Structural Formulae (200) to (275) below.

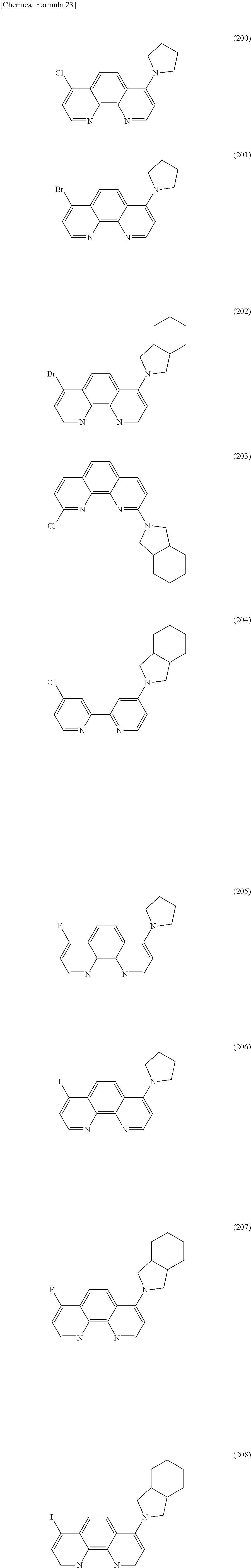

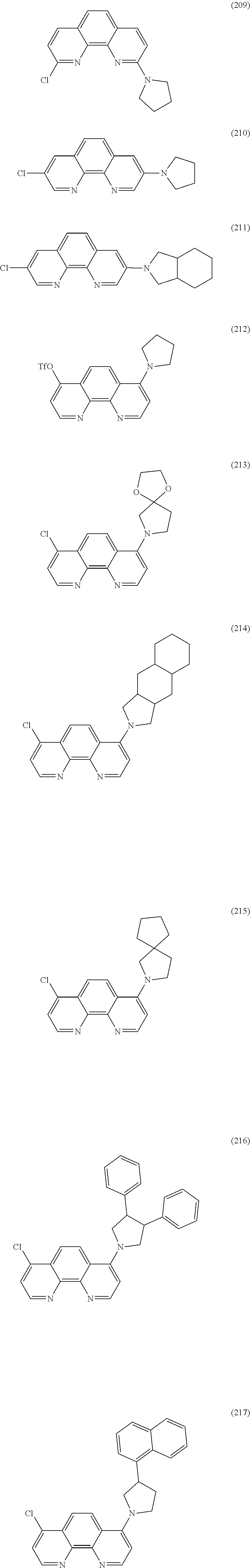

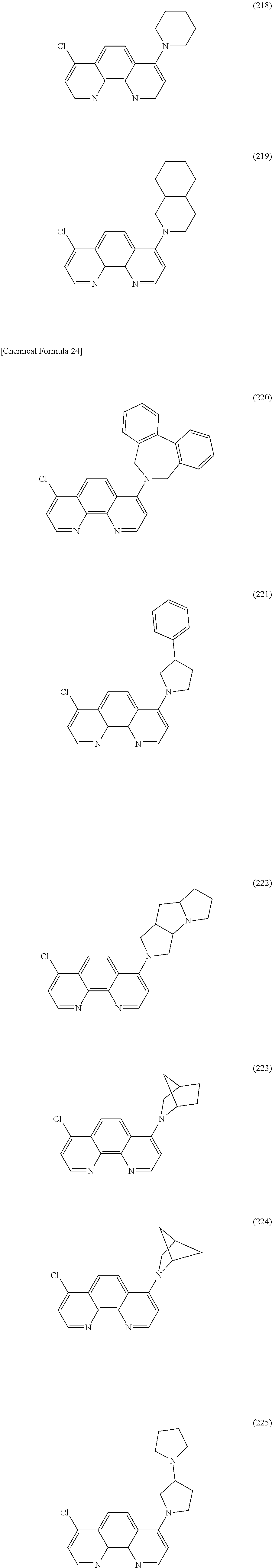

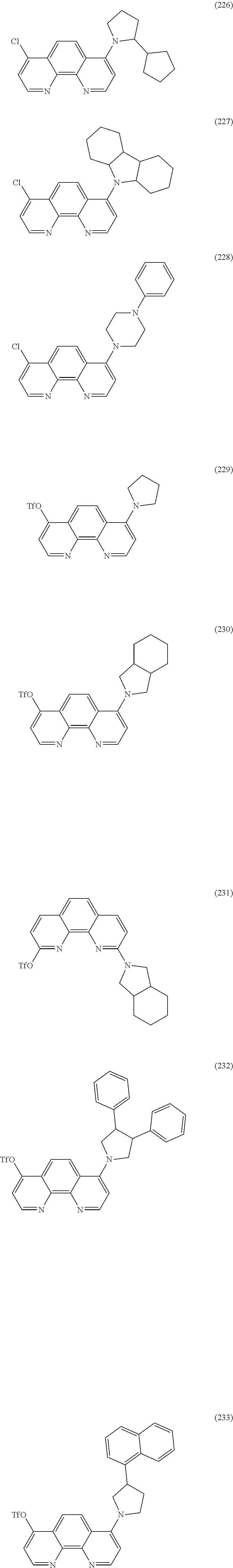

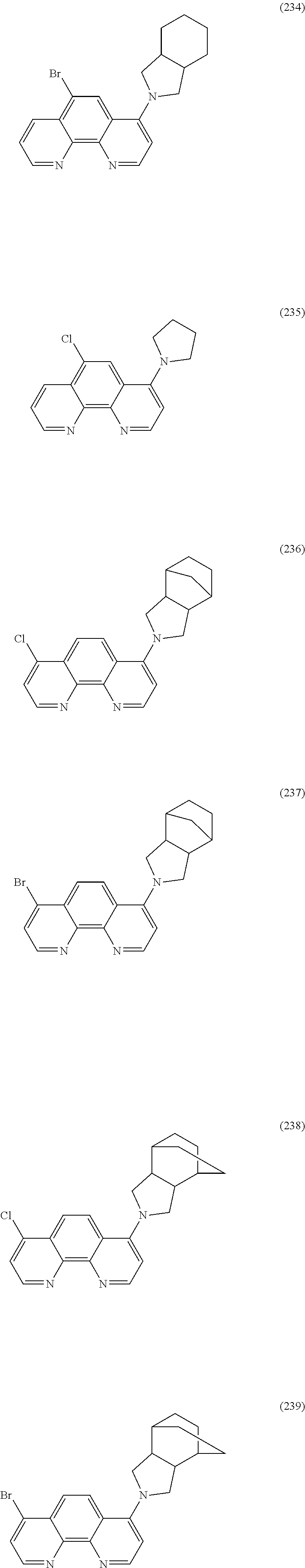

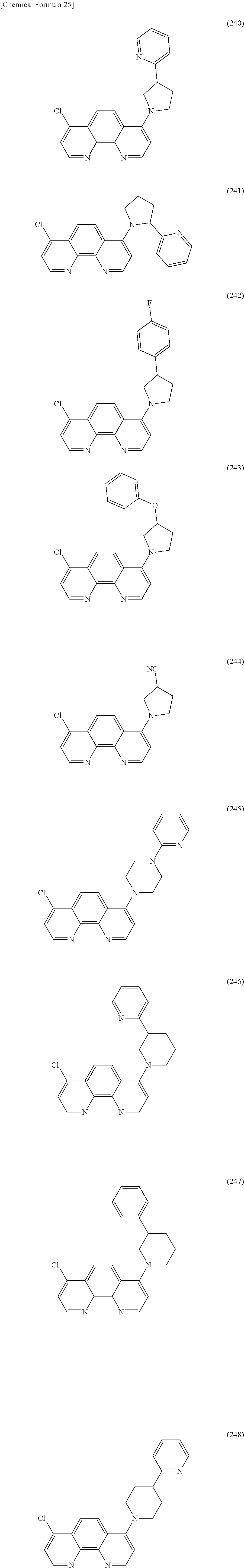

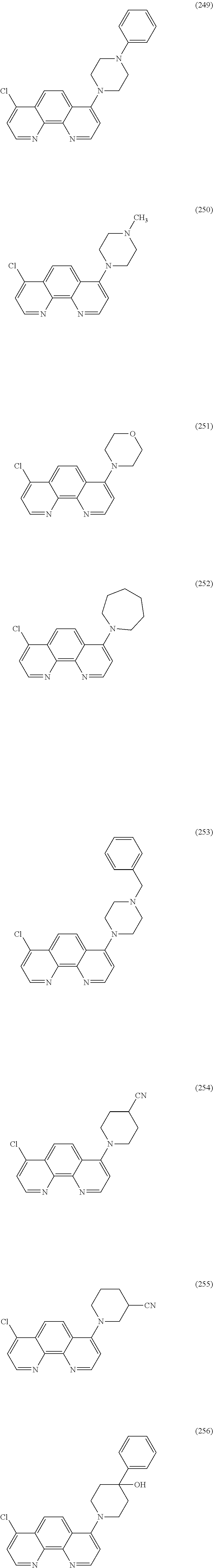

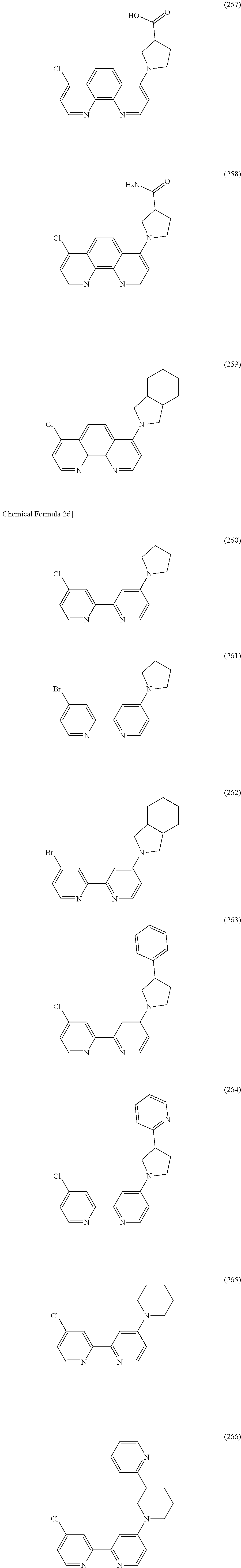

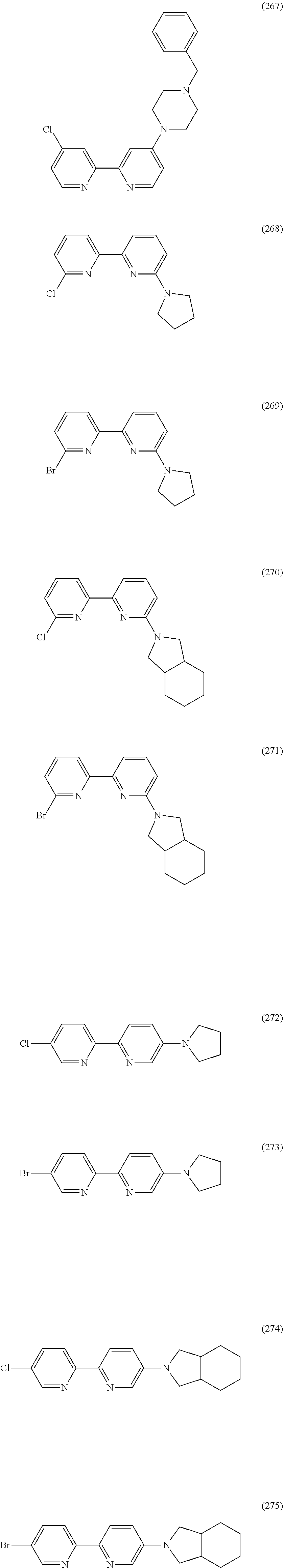

Examples of the organic compound that is represented by General Formula (G3) above and can be synthesized using the organic compound represented by General Formula (G1) above include organic compounds represented by Structural Formulae (100) to (135) below.

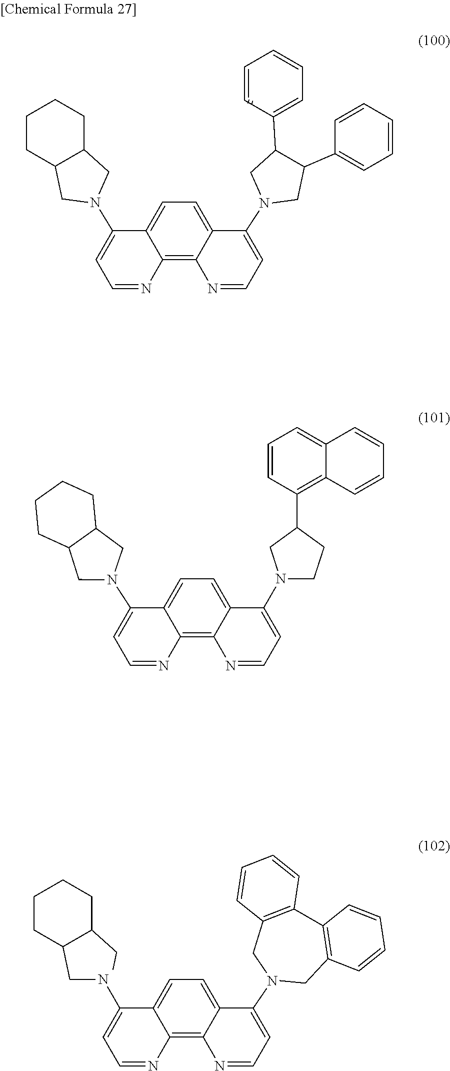

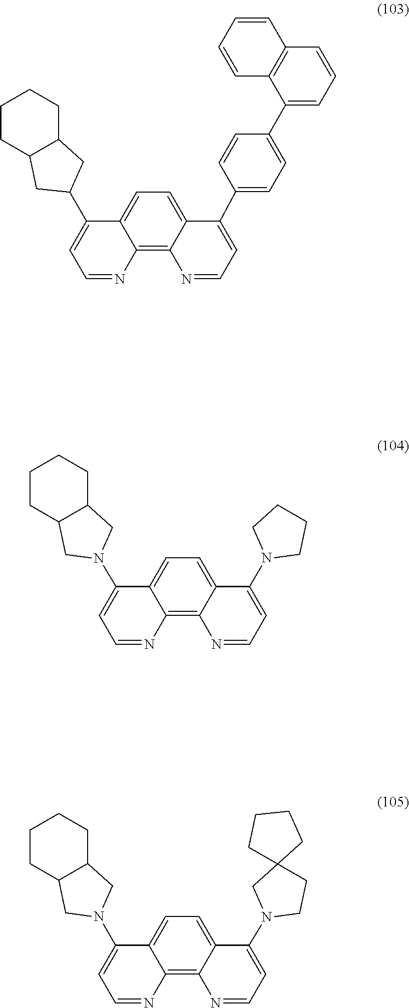

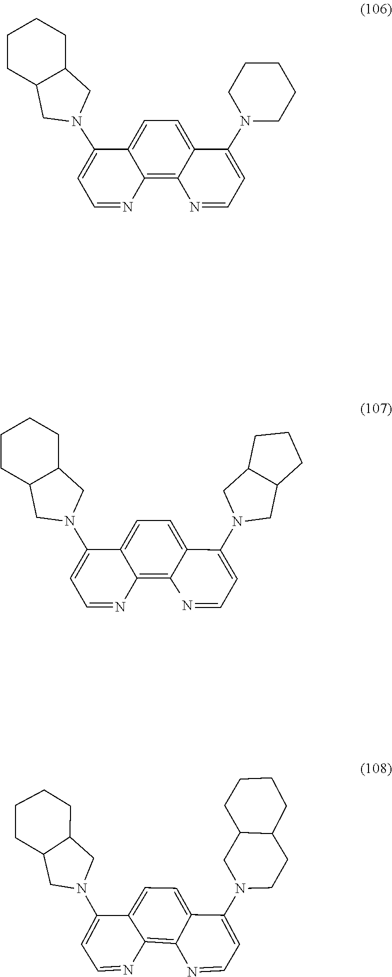

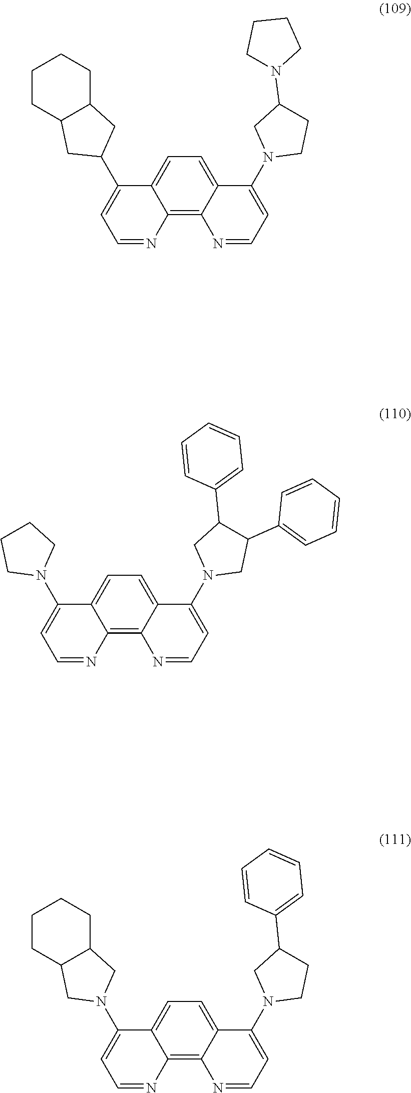

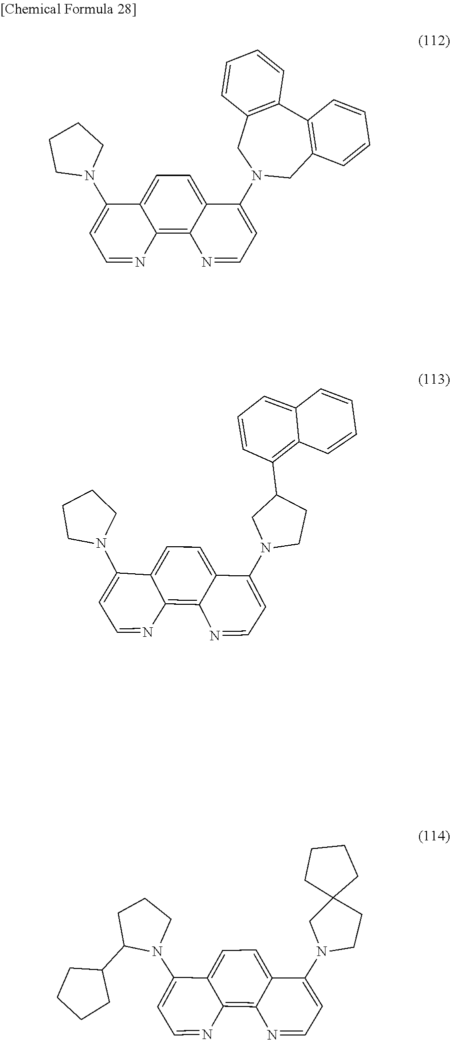

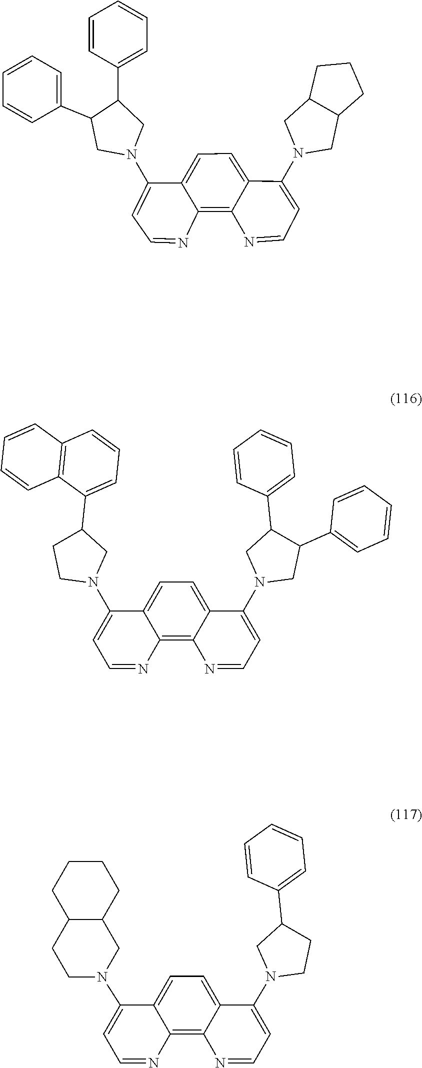

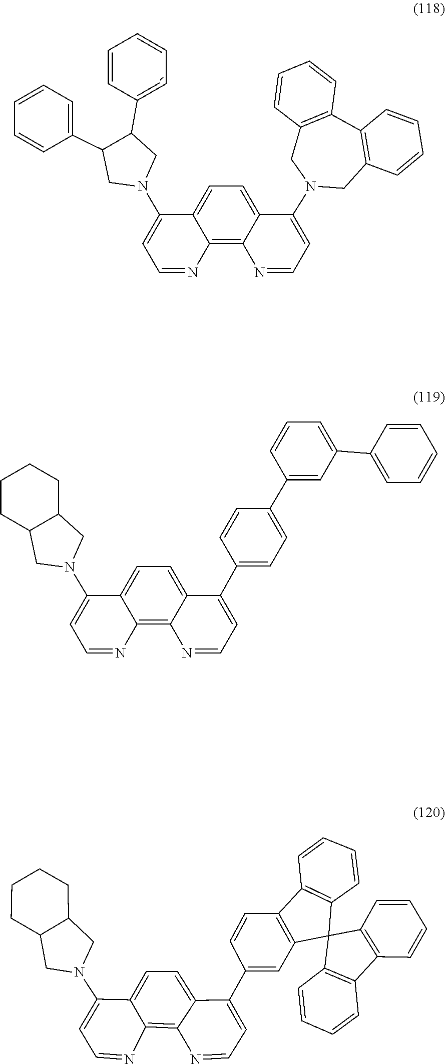

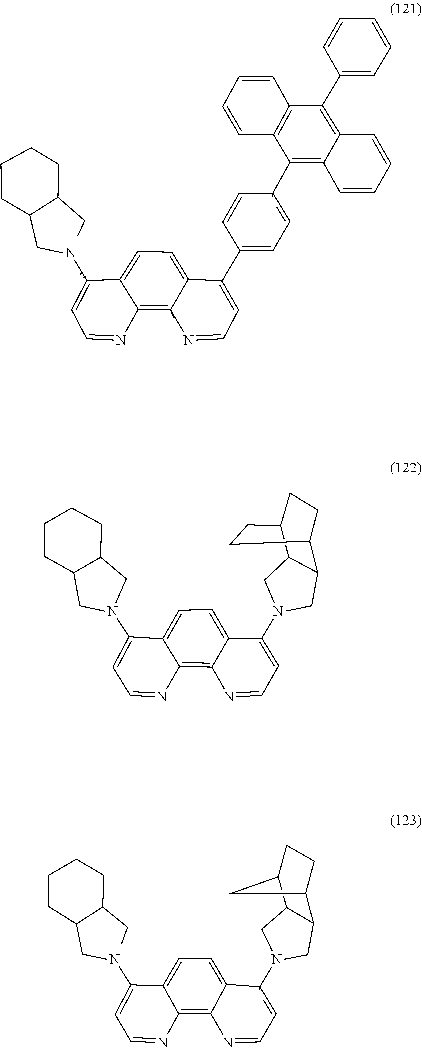

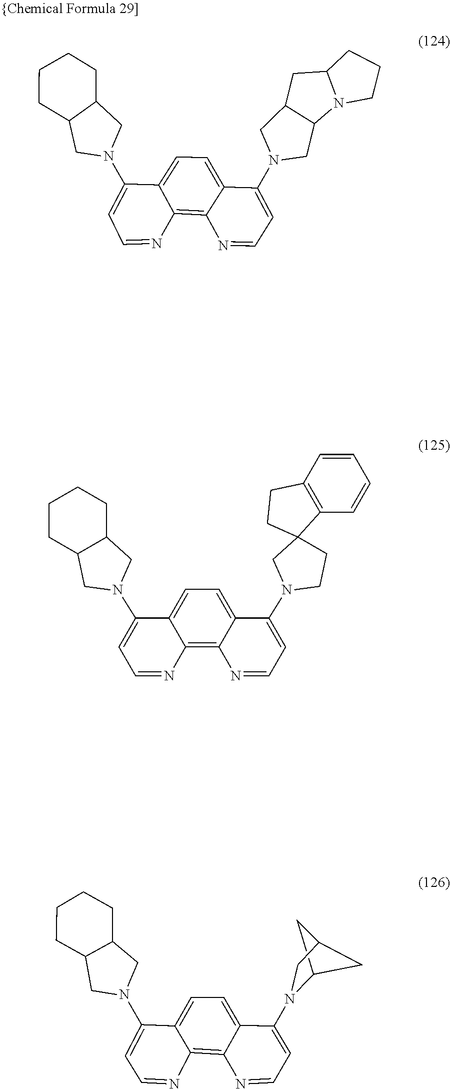

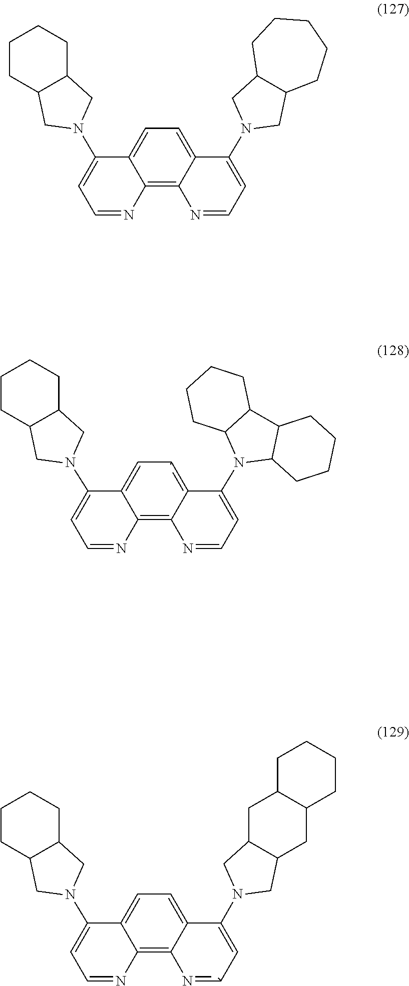

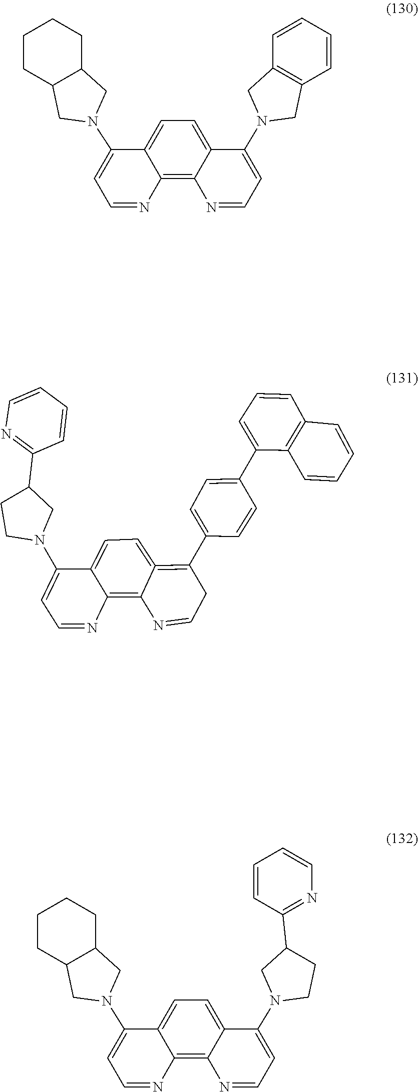

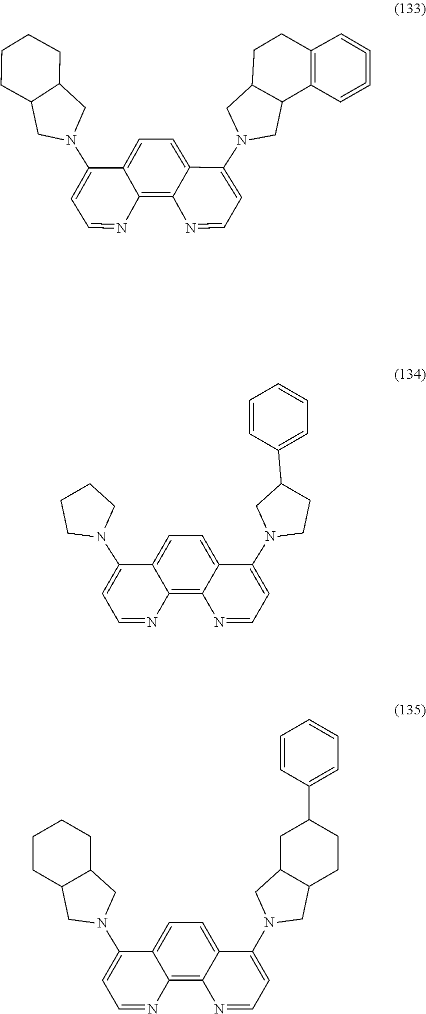

A method for synthesizing the above-described organic compound represented by any of General Formulae (G1) to (G3), which is the organic compound of one embodiment of the present invention, will be described in detail below.

First, a method for synthesizing the organic compound represented by General Formula (G1) or (G2) above, which is the organic compound of one embodiment of the present invention, will be described using an example of synthesizing an organic compound represented by General Formula (G1-3-1) below. Note that General Formula (G1-3-1) below is a general formula showing a state where R in General Formula (G1-3) above is substituted with the group represented by (g1). Note that X, R¹¹ to R¹⁸, p, and q in General Formula (G1-3-1) are similar to those in (G1-3), and thus repeated description thereof is omitted.

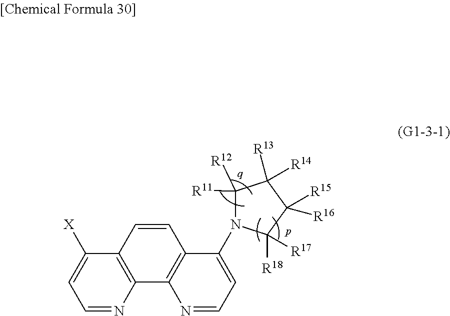

The organic compound represented by General Formula (G1-3-1) can be synthesized by a simple synthesis scheme such as Synthesis Scheme (A-1) below.

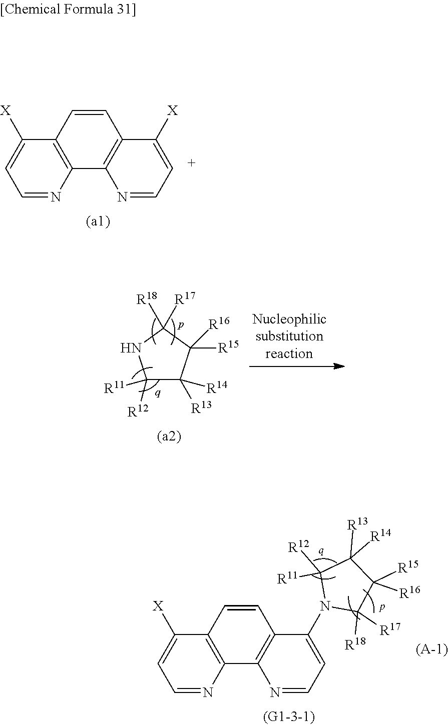

In the above compound (a1), X represents a halogen or a trifluoromethanesulfonyl group.

In the above compound (a2), R¹¹ to R¹⁸, p, and q are similar to those in General Formula (g1).

In Synthesis Scheme (A-1), a phenanthroline derivative (a1) and an aliphatic cyclic amine derivative (a2) are subjected to a nucleophilic substitution reaction using an appropriate solvent and an appropriate inorganic base, whereby a phenanthroline intermediate having the aliphatic cyclic amino group as represented by General Formula (G1-3) can be obtained.

Examples of inorganic bases that can be used in the nucleophilic substitution reaction represented by Synthesis Scheme (A-1) above include carbonates such as potassium carbonate, cesium carbonate, sodium carbonate, and potassium hydrogen carbonate, acetates such as potassium acetate and sodium acetate, and phosphates such as tripotassium phosphate and trisodium phosphate. Note that potassium carbonate and potassium acetate, especially potassium carbonate, are preferable to achieve high yield.

Examples of solvents that can be used in the nucleophilic substitution reaction represented by Synthesis Scheme (A-1) above include N-methyl-2-pyrrolidone, N,N-dimethylformamide, tetrahydrofuran, dioxane, ethanol, ethyl acetate, and toluene. However, the solvents that can be used are not limited to these solvents.

The method for synthesizing the organic compound represented by General Formula (G1-3-1) is described above, and the organic compounds represented by General Formulae (G1-1), (G1-2), (G1-4), and (G2-1) to (G2-4) can also be synthesized by changing the phenanthroline derivative (a1) to corresponding raw materials.

Next, a method for synthesizing the organic compound represented by General Formula (G3) will be described using an example of an organic compound represented by General Formula (3-1).

The organic compound represented by General Formula (G3-1), in which the substituent A in the organic compound represented by General Formula (G3) above is the group represented by General Formula (g2) above, can be synthesized by a simple synthesis scheme such as Synthesis Scheme (A-1) above or Synthesis Scheme (A-2) below.

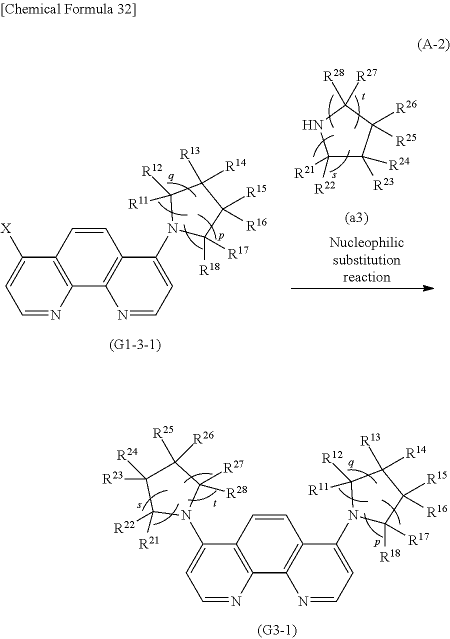

In the above compound (a3), R²¹ to R²⁸, s, and t are similar to those in General Formula (g2). Note that a compound different from the compound (a2) is used.

In Synthesis Scheme (A-2), the phenanthroline intermediate having the aliphatic cyclic amino group as represented by General Formula (G1-3) and an aliphatic cyclic amine derivative (a3) are subjected to a nucleophilic substitution reaction using an appropriate solvent and an appropriate base, whereby the organic compound represented by General Formula (G3-1) can be obtained.

Examples of bases that can be used in the nucleophilic substitution reaction represented by Synthesis Scheme (A-2) above include organic bases such as diazabicycloundecene (DBU), triethylamine, and potassium tert-butoxide and inorganic bases such as potassium carbonate, cesium carbonate, sodium carbonate, sodium hydrogen carbonate, potassium acetate, sodium acetate, tripotassium phosphate, and trisodium phosphate.

Examples of solvents that can be used in the nucleophilic substitution reaction represented by Synthesis Scheme (A-2) above include N-methyl-2-pyrrolidone, N,N-dimethylformamide, toluene, tetrahydrofuran, dioxane, and ethanol. However, the solvents that can be used are not limited to these solvents. An organic base may be used as both the base and the solvent.

The reaction in Synthesis Scheme (A-2) above is not limited to the nucleophilic substitution reaction and can be a Buchwald-Hartwig reaction, a coupling reaction using copper or a copper compound, or the like.

The organic compound represented by General Formula (G3-2), in which the substituent A in the organic compound represented by General Formula (G3) above is the group represented by General Formula (g3) above, can be synthesized by a simple synthesis scheme such as Synthesis Scheme (A-1) above or Synthesis Scheme (A-3) below.

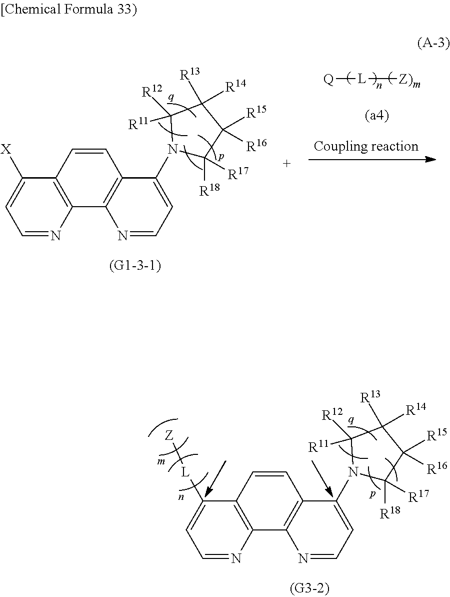

In the above compound (a4), Q represents a boronyl group (—B(OH)₂), and Z, L, n, and m are similar to those in General Formula (g3). In the case where Q is a boronyl group in the compound (a4), a boronic ester, a cyclic-triolborate salt, or the like may be used. Note that Q is not necessarily limited to a boronyl group. A general and common nucleophile that can cause a coupling reaction, such as magnesium or zinc, may be used. A boronyl group is preferable in terms of environmental burden and ease of raw material procurement.

Examples of palladium catalysts that can be used in the coupling reaction represented by Synthesis Scheme (A-3) above include palladium(II) acetate, tetrakis(triphenylphosphine)palladium(0), and bis(triphenylphosphine)palladium(II) dichloride. Examples of ligands in palladium catalysts include di(1-adamanthyl)-n-butylphosphine, (±)-2,2′-bis(diphenylphosphino)-1,1′-binaphthyl, tri(ortho-tolyl)phosphine, triphenylphosphine, and tricyclohexylphosphine.

Examples of bases that can be used in the coupling reaction represented by Synthesis Scheme (A-3) above include organic bases such as potassium tert-butoxide and inorganic bases such as cesium carbonate, potassium carbonate, sodium carbonate, and tripotassium phosphate.

Examples of solvents that can be used in the coupling reaction represented by the above synthesis scheme include 1,2-dimethoxyethane, toluene, xylene, mesitylene, benzene, tetrahydrofuran, and dioxane. However, the solvents that can be used are not limited to these solvents.

The reaction in Synthesis Scheme (A-3) above is not limited to a Suzuki-Miyaura reaction and can be a Migita-Kosugi-Stille coupling reaction using an organotin compound, a coupling reaction using a Grignard reagent, a coupling reaction using a Negishi reaction and copper or a copper compound, a nucleophilic substitution reaction, or the like.

A variety of kinds of the above compounds (a1) to (a4) are commercially available or can be synthesized.

The organic compound of one embodiment of the present invention can be synthesized in the above manner, but the present invention is not limited to this and other synthesis methods may be employed.

This embodiment can be freely combined with any of the other embodiments and the examples.

This embodiment can be combined as appropriate with the other embodiments or the examples. In this specification, in the case where a plurality of structure examples are shown in one embodiment, the structure examples can be combined as appropriate.

### Embodiment 2

In this embodiment, light-emitting devices of one embodiment of the present invention will be described in detail.

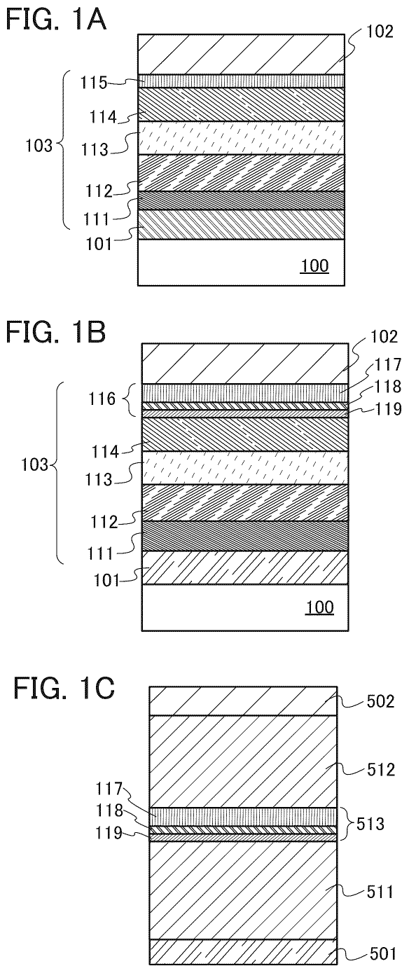

FIGS. 1A to 1C are schematic diagrams of light-emitting devices of one embodiment of the present invention. Each of the light-emitting devices includes a first electrode 101 over an insulator 100, and an organic compound layer 103 between the first electrode 101 and a second electrode 102. The organic compound layer 103 includes at least one of the organic compounds represented by General Formula (G3) in Embodiment 1. A light-emitting layer 113 in the light-emitting device contains an emission center substance that emits light when voltage is applied between the first electrode 101 and the second electrode 102.

The organic compound layer 103 preferably includes, besides the light-emitting layer 113, functional layers such as a hole-injection layer 111, a hole-transport layer 112, an electron-transport layer 114, and an electron-injection layer 115, as illustrated in FIG. 1A. Note that the organic compound layer 103 may include functional layers other than the above functional layers, such as a hole-blocking layer, an electron-blocking layer, an exciton-blocking layer, and a charge-generation layer. Alternatively, any of the above layers may be omitted.

Since the organic compound represented by General Formula (G3) in Embodiment 1 has a high electron-transport property and a high electron-injection property, the organic compound is preferably contained in a layer where electrons serve as carriers. Examples of the layer where electrons serve as carriers include an electron-injection layer, an electron-transport layer, a hole-blocking layer, a light-emitting layer, and an intermediate layer; the organic compound represented by General Formula (G3) is preferably used in the electron-injection layer or the intermediate layer, in particular.

Although the first electrode 101 includes an anode and the second electrode 102 includes a cathode in this embodiment, the first electrode 101 may include a cathode and the second electrode 102 may include an anode. The first electrode 101 and the second electrode 102 each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability.

The anode is preferably formed using a metal, an alloy, a conductive compound, or a mixture thereof each having a high work function (specifically, higher than or equal to 4.0 eV), for example. Specific examples include indium oxide-tin oxide (ITO: indium tin oxide), indium oxide-tin oxide containing silicon or silicon oxide (ITSO: indium tin silicon oxide), indium oxide-zinc oxide, and indium oxide containing tungsten oxide and zinc oxide (IWZO). Films of such conductive metal oxides are usually formed by a sputtering method, but may be formed by a sol-gel method or the like. For example, a film of indium oxide-zinc oxide is formed by a sputtering method using a target in which 1 wt % to 20 wt % zinc oxide is added to indium oxide. Furthermore, a film of indium oxide containing tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), titanium (Ti), aluminum (Al), a nitride of a metal material (e.g., titanium nitride), or the like can be used for the anode. The anode may be a stack of layers formed of any of these materials. For example, a film in which Al, Ti, and ITSO are stacked in this order over Ti is preferable because the film has high efficiency owing to high reflectivity and enables a high resolution of several thousand ppi. Graphene can also be used for the anode. Note that an electrode material can be selected regardless of the work function when the composite material that can be used for the hole-injection layer 111 described below is used for the layer (typically the hole-injection layer) in contact with the anode.

The hole-injection layer 111 is provided in contact with the anode and has a function of facilitating injection of holes into the organic compound layer 103. The hole-injection layer 111 can be formed using a phthalocyanine compound or a phthalocyanine-based complex compound such as phthalocyanine (abbreviation: H₂Pc) or copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N′-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS), for example.

The hole-injection layer 111 may be formed using a substance having an electron-acceptor property. Examples of the substance having an acceptor property include organic compounds having an electron-withdrawing group (e.g., a halogen group or a cyano group), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a significantly high electron-acceptor property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds.

The hole-injection layer 111 is preferably formed using a composite material containing any of the aforementioned materials having an acceptor property and an organic compound having a hole-transport property.

As the organic compound having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the organic compound having a hole-transport property used for the composite material preferably has a hole mobility of 1×10⁻⁶ cm²/Vs or higher. The organic compound having a hole-transport property used in the composite material is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton in the ring is preferable; specifically, a carbazole ring, a dibenzothiophene ring, or a ring in which an aromatic ring or a heteroaromatic ring is further condensed to a carbazole ring or a dibenzothiophene ring is preferable.

Such an organic compound having a hole-transport property further preferably has at least one of a carbazole skeleton, a dibenzofuran skeleton, a dibenzothiophene skeleton, and an anthracene skeleton. In particular, an aromatic amine having a substituent that has a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable fabricating a light-emitting device having a long lifetime.

Specific examples of the organic compound having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Examples of the aromatic amine compounds that can be used as the material having a hole-transport property include N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

The formation of the hole-injection layer 111 can improve the hole-injection property, which allows the light-emitting device to be driven at a low voltage.

Among substances having an acceptor property, an organic compound having an acceptor property is easy to use because the organic compound is easily deposited by evaporation as a film.

The hole-transport layer 112 is formed using an organic compound having a hole-transport property. The organic compound having a hole-transport property preferably has a hole mobility of 1×10⁻⁶ cm²/Vs or higher.

Examples of the aforementioned material having a hole-transport property include the following compounds: compounds having an aromatic amine skeleton, such as 4,4′-bis[N-(1-naphthyl)-N-phenylamino]biphenyl (abbreviation: NPB), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-3,3′-bi-9H-carbazole (abbreviation: βNCCP), 9-(3-biphenyl)-9′-(2-naphthyl)-3,3′-bi-9H-carbazole (abbreviation: PNCCmBP), 9-(4-biphenyl)-9′-(2-naphthyl)-3,3′-bi-9H-carbazole (abbreviation: PNCCBP), 9,9′-di-2-naphthyl-3,3′-9H,9′H-bicarbazole (abbreviation: BisPNCz), 9-(2-naphthyl)-9′-[1,1′:4′,1″-terphenyl]-3-yl-3,3′-9H,9′H-bicarbazole, 9-(2-naphthyl)-9′-[1,1′: 3′,1″-terphenyl]-3-yl-3,3′-9H,9′H-bicarbazole, 9-(2-naphthyl)-9′-[1,1′: 3′,1″-terphenyl]-5′-yl-3,3′-9H,9′H-bicarbazole, 9-(2-naphthyl)-9′-[1,1′: 4′,1″-terphenyl]-4-yl-3,3′-9H,9′H-bicarbazole, 9-(2-naphthyl)-9′-[1,1′: 3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′: 3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have a high hole-transport property to contribute to a reduction in driving voltage. Note that any of the substances given as examples of the material having a hole-transport property that is used for the composite material in the hole-injection layer 111 can also be suitably used as the material included in the hole-transport layer 112.

The emission center substance may be a fluorescent substance, a phosphorescent substance, a substance exhibiting thermally activated delayed fluorescence (TADF), or any other light-emitting substance.

Examples of materials that can be used as the fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N″,N″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPm, and 1,6BnfAPrn-03 are particularly preferable because of their high hole-trapping properties, high emission efficiency, or high reliability.

A condensed heteroaromatic compound containing nitrogen and boron, especially a compound having a diaza-boranaphtho-anthracene skeleton, exhibits a narrow emission spectrum, emits blue light with high color purity, and can thus be suitably used. Examples of the compound include 5,9-diphenyl-5H,9H-[1,4]benzazaborino[2,3,4-kl]phenazaborine (abbreviation: DABNA-1), 9-(diphenyl-3-yl)-N,N,5,11-tetraphenyl-5,9-dihydro-5,9-diaza-13b-boranaphtho[3,2,1-de]anthracen-3-amine (abbreviation: DABNA-2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′,3′,2′: 4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-Y), or the like can be suitably used.

In the case where a phosphorescent substance is used as the light-emitting substance in the light-emitting layer, a metal complex, in particular, an iridium complex or a platinum complex is preferable as the phosphorescent substance; examples of materials are as follows.

The examples include organometallic iridium complexes having a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), and tris(5-methyl-3,4-diphenyl-4H-1,2,4-triazolato)iridium(III) (abbreviation: [Ir(Mptz)₃]); organometallic iridium complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); organometallic complexes having a benzimizazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); and organometallic iridium complexes in which a phenylpyridine derivative having an electron-withdrawing group is a ligand, such as bis[2-(4′,6′-difluorophenyl)pyridinato-N,C²′]iridium(III) tetrakis(1-pyrazolyl)borate (abbreviation: FIr6), bis[2-(4′,6′-difluorophenyl)pyridinato-N,C²]iridium(III) picolinate (abbreviation: FIrpic), bis{2-[3′,5′-bis(trifluoromethyl)phenyl]pyridinato-N,C²′}iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIracac). These compounds emit blue phosphorescent light and have an emission peak in the wavelength range from 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), bis(2-phenylquinolinato-N,C²)iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: [Ir(5mppy-d₃)₂(mbfpypy-d₃)]), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: [Ir(5mtpy-d₆)₂(mbfpypy-iPr-d₄)]), [2-(methyl-d₃)-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy-d₃)]), [2-(4-methyl-5-phenyl-2-pyridinyl-κN)phenyl-κC]bis[2-(2-pyridinyl-κN)phenyl-κC]iridium(III) (abbreviation: [Ir(ppy)₂(mdppy)]), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), and [2-methyl-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]); organometallic platinum complexes such as (2-{1-(5-tert-butylbiphenyl-2-yl)-4-[3-tert-butyl-5-(4-phenyl-2-pyridinyl-κN)phenyl-κC⁶]-2-benzimidazolyl-κN³}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]}-2′-yl)-2-pyridinyl-κN]phenyl-κC²}-2-pyridinyl-κN)phenolato-κO]platinum(II) (abbreviation: Pt(4tButpppypyp-mmtBup)); and rare earth metal complexes such as tris(acetylacetonato)(monophenanthroline)terbium(III) (abbreviation: [Tb(acac)₃(Phen)]). These compounds mainly emit green phosphorescent light and have an emission peak in the wavelength range from 500 nm to 600 nm. Note that organometallic iridium complexes having a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C²)iridium(III) (abbreviation: [Ir(piq)₃]), bis(1-phenylisoquinolinato-N,C²)iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-N]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit red phosphorescent light and have an emission peak in the wavelength range from 600 nm to 700 nm. Furthermore, the organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

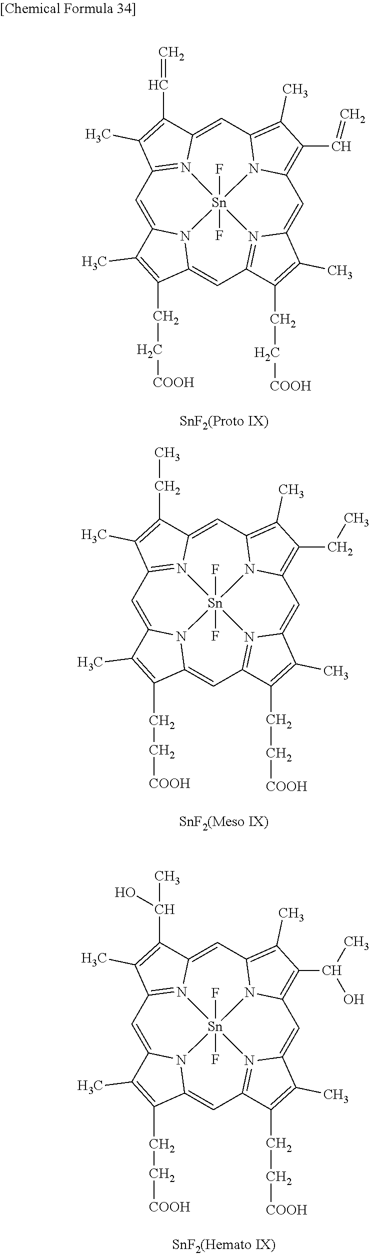

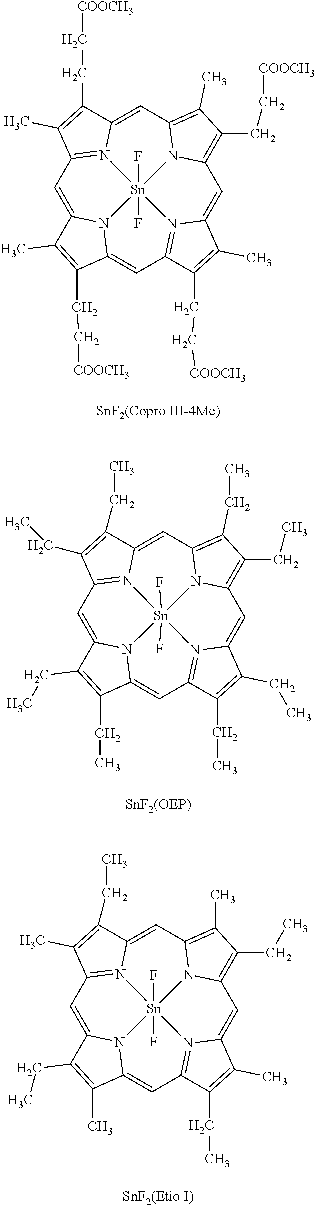

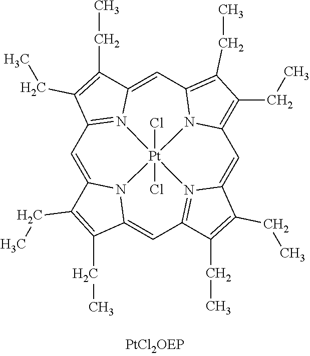

Alternatively, it is possible to use a heterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferable because the electron-donating property of the π-electron rich heteroaromatic ring and the electron-acceptor property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

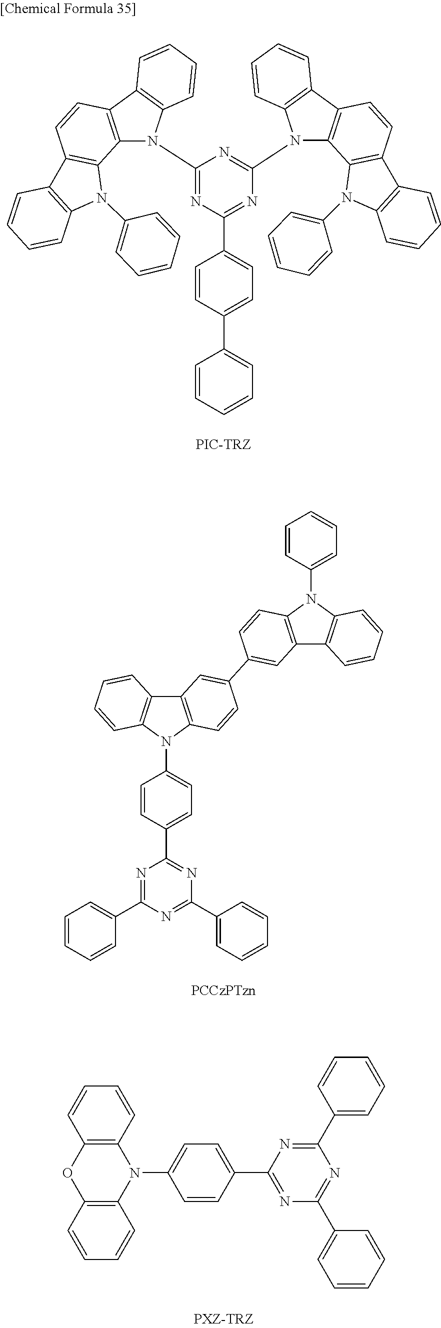 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(N4c5ccccc5Oc5ccccc54)cc3)n2)cc1]

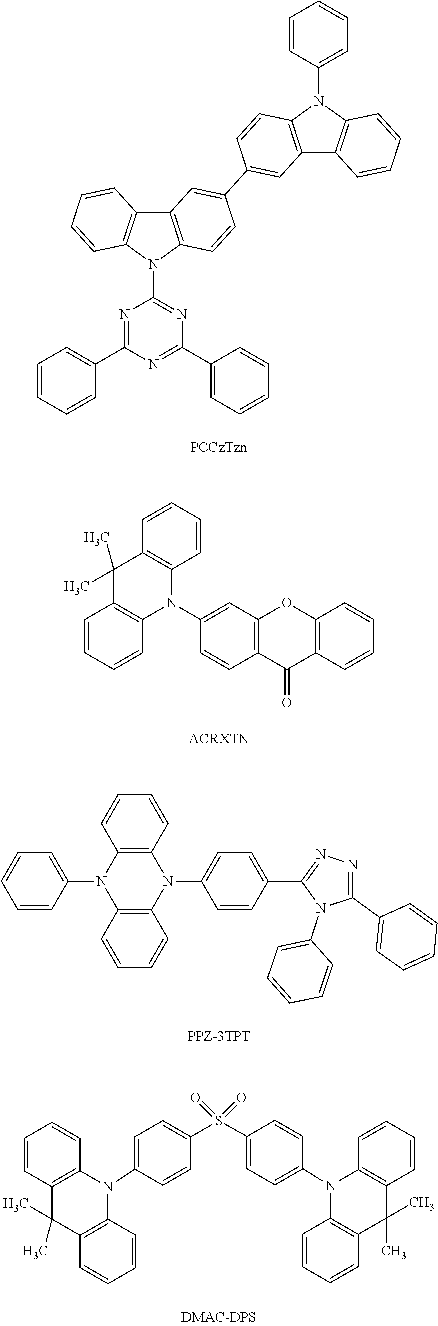 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; CC1(C)c2ccccc2N(c2ccc3c(=O)c4ccccc4oc3c2)c2ccccc21 ; c1ccc(-c2nnc(-c3ccc(N4c5ccccc5N(c5ccccc5)c5ccccc54)cc3)n2-c2ccccc2)cc1 ; CC1(C)c2ccccc2N(c2ccc(S(=O)(=O)c3ccc(N4c5ccccc5C(C)(C)c5ccccc54)cc3)cc2)c2ccccc21]

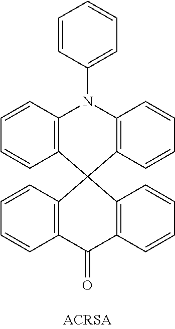 [SMILES: O=C1c2ccccc2C2(c3ccccc31)c1ccccc1N(c1ccccc1)c1ccccc12]

Note that a TADF material is a material having a small difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed of two kinds of substances has an extremely small difference between the S₁ level and the T₁ level and functions as a TADF material capable of converting triplet excitation energy into singlet excitation energy.

A phosphorescence spectrum observed at a low temperature (e.g., 77 K to 10 K) is used for an index of the T₁ level. When the level of energy with a wavelength of a line obtained by extrapolating a tangent to the fluorescence spectrum at a tail on the short wavelength side is the S₁ level and the level of energy with a wavelength of the line obtained by extrapolating a tangent to the phosphorescence spectrum at a tail on the short wavelength side is the T₁ level, the difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, various carrier-transport materials such as materials having an electron-transport property and/or materials having a hole-transport property, and the TADF materials can be used.

The material having a hole-transport property is preferably an organic compound having an amine skeleton or a π-electron rich heteroaromatic ring skeleton, for example. As the π-electron rich heteroaromatic ring, a condensed aromatic ring having at least one of an acridine skeleton, a phenoxazine skeleton, a phenothiazine skeleton, a furan skeleton, a thiophene skeleton, and a pyrrole skeleton is preferable; specifically, a carbazole ring, a dibenzothiophene ring, or a ring in which an aromatic ring or a heteroaromatic ring is further condensed to a carbazole ring or a dibenzothiophene ring is preferable.

Such an organic compound having a hole-transport property further preferably has at least one of a carbazole skeleton, a dibenzofuran skeleton, a dibenzothiophene skeleton, and an anthracene skeleton. In particular, an aromatic amine having a substituent that has a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable fabricating a light-emitting device having a long lifetime.

Examples of such organic compounds include the following compounds: compounds having an aromatic amine skeleton, such as 4,4′-bis[N-(1-naphthyl)-N-phenylamino]biphenyl (abbreviation: NPB), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have a high hole-transport property to contribute to a reduction in driving voltage. In addition, the organic compounds given as examples of the material having a hole-transport property that can be used for the hole-transport layer can also be used.

The material having an electron-transport property preferably has an electron mobility higher than or equal to 1×10⁻⁷ cm²/Vs, further preferably higher than or equal to 1×10⁻⁶ cm²/Vs when the square root of the electric field strength [V/cm] is 600. Note that any other substance can also be used as long as the substance has a property of transporting more electrons than holes.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis [2-(2-benzothiazolyl)phenolato]zinc(II) (abbreviation: ZnBTZ); or an organic compound having a π-electron deficient heteroaromatic ring is preferably used. Examples of the organic compound having a π-electron deficient heteroaromatic ring skeleton include an organic compound that has a heteroaromatic ring having an azole skeleton, an organic compound that has a heteroaromatic ring having a pyridine skeleton, an organic compound that has a heteroaromatic ring having a diazine skeleton, and an organic compound that has a heteroaromatic ring having a triazine skeleton.

Among the above organic compounds, the organic compound that has a heteroaromatic ring having a diazine skeleton (a pyrimidine skeleton, a pyrazine skeleton, or a pyridazine skeleton), the organic compound that has a heteroaromatic ring having a pyridine skeleton, and the organic compound that has a heteroaromatic ring having a triazine skeleton are preferable because of their high reliability. In particular, the organic compound that has a heteroaromatic ring having a diazine (pyrimidine or pyrazine) skeleton and the organic compound that has a heteroaromatic ring having a triazine skeleton have a high electron-transport property to contribute to a reduction in driving voltage. A benzofuropyrimidine skeleton, a benzothienopyrimidine skeleton, a benzofuropyrazine skeleton, and a benzothienopyrazine skeleton are preferable because of their high acceptor properties and high reliability.

Examples of the organic compound having a π-electron deficient heteroaromatic ring skeleton include organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOS); organic compounds that have a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-[3-(2-triphenylenyl)phenyl]-1,10-phenanthroline (abbreviation: mTpPPhen), 2-phenyl-9-(2-triphenylenyl)-1,10-phenanthroline (abbreviation: Ph-TpPhen), 2-[4-(9-phenanthryl)-1-naphthyl]-1,10-phenanthroline (abbreviation: PnNPhen), and 2-[4-(2-triphenylenyl)phenyl]-1,10-phenanthroline (abbreviation: pTpPPhen); organic compounds having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′:4,5]furo[3,2-d]pyrimidine (abbreviation: 8mDBtBPNfpm), 8-([2,2′-binaphthalen]-6-yl)-4-[3-(dibenzothiophen-4-yl)phenyl]-[1]benzofuro[3,2-d]pyrimidine (abbreviation: 8(βN2)-4mDBtPBfpm), 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 2,2′-(pyridine-2,6-diyl)bis(4-phenylbenzo[h]quinazoline) (abbreviation: 2,6(P-Bqn)2Py), 2,2′-([2,2′-bipyridine]-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), 7-[4-(9-phenyl-9H-carbazol-2-yl)quinazolin-2-yl]-7H-dibenzo[c,g]carbazole (abbreviation: PC-cgDBCzQz), and 11-[3′-(dibenzothiophen-4-yl)biphenyl-3-yl]phenanthro[9′,10′:4,5]furo[2,3-b]pyrazine (abbreviation: 11mDBtBPPnfpr); and organic compounds that have a heteroaromatic ring having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tzn), 2-[3-(2,6-dimethyl-3-pyridinyl)-5-(9-phenanthryl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-[8-([1,1′: 4′,1″-terphenyl]-4-yl)-1-dibenzofuranyl]-1,3,5-triazine (abbreviation: mBP-TPDBfTzn), 2-[4-(2-naphthyl)phenyl]-4-phenyl-6-spiro[9H-fluorene-9,9′-[9H]xanthen]-4-yl-1,3,5-triazine (abbreviation: PNP-SFx(4)Tzn), 2-phenyl-4,6-bis[3-(triphenylsilyl)phenyl]-1,3,5-triazine (abbreviation: mSiTrz), and 9,9′-{6-[3-(triphenylsilyl)phenyl]-1,3,5-triazine-2,4-diyl}bis(9H-carbazole) (abbreviation: SiTrzCz2). The organic compound that has a heteroaromatic ring having a diazine skeleton, the organic compound that has a heteroaromatic ring having a pyridine skeleton, and the organic compound that has a heteroaromatic ring having a triazine skeleton are preferable because of their high reliability. In particular, the organic compound that has a heteroaromatic ring having a diazine (pyrimidine or pyrazine) skeleton and the organic compound that has a heteroaromatic ring having a triazine skeleton have a high electron-transport property to contribute to a reduction in driving voltage.

The organic compound represented by General Formula (G3) in Embodiment 1 also has a pyridine skeleton and a π-electron deficient heteroaromatic ring skeleton, and thus can be suitably used as the material having an electron-transport property.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material that emits light whose wavelength overlaps with the wavelength of the lowest-energy-side absorption band of the fluorescent substance. This enables smooth transfer of excitation energy from the TADF material to the fluorescent substance and accordingly enables efficient light emission, which is preferable.

In order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton that brings about light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transportation or carrier recombination. Here, the luminophore refers to an atomic group (skeleton) that brings about light emission in a fluorescent substance. The luminophore is preferably a skeleton having a π bond, further preferably has an aromatic ring, and still further preferably has a condensed aromatic ring or a condensed heteroaromatic ring. Examples of the luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an acene skeleton, especially an anthracene skeleton, is suitably used as the host material. The use of a substance having an anthracene skeleton as the host material for the fluorescent substance makes it possible to obtain a light-emitting layer with high emission efficiency and high durability. Among the substances having an anthracene skeleton that are used as the host materials, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to a carbazole skeleton because the HOMO level thereof is higher than that of the host material having a carbazole skeleton by approximately 0.1 eV and thus holes enter the host material easily. In particular, the host material preferably has a dibenzocarbazole skeleton because the HOMO level thereof is higher than that of the host material having a carbazole skeleton by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Furthermore, a dibenzofuran skeleton is preferably included, in which case the reliability can be ensured without a reduction in the T₁ level.

Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,βADN), 2-(10-phenylanthracen-9-yl)dibenzofuran, 2-(10-phenyl-9-anthryl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthryl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

These mixed materials may form an exciplex. These mixed materials are preferably selected so as to form an exciplex that exhibits light emission whose wavelength overlaps with the wavelength of the lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the LUMO level of the material having a hole-transport property is preferably higher than or equal to that of the material having an electron-transport property. Note that the LUMO levels and the HOMO levels of the materials can be calculated from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient photoluminescence (PL) lifetime of the mixed film has longer lifetime components or has a larger proportion of delayed components than that of each of the materials, observed by comparison of transient PL of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

The electron-transport layer 114 contains a material having an electron-transport property. The material having an electron-transport property preferably has an electron mobility higher than or equal to 1×10⁻⁷ cm²/Vs, further preferably higher than or equal to 1×10⁻⁶ cm²/Vs when the square root of the electric field strength [V/cm] is 600. Note that any other substance can also be used as long as the substance has a property of transporting more electrons than holes. The above organic compound is preferably an organic compound that has a π-electron deficient heteroaromatic ring. The organic compound that has a π-electron deficient heteroaromatic ring is preferably one or more of an organic compound that has a heteroaromatic ring having an azole skeleton, an organic compound that has a heteroaromatic ring having a pyridine skeleton, an organic compound that has a heteroaromatic ring having a diazine skeleton, and an organic compound that has a heteroaromatic ring having a triazine skeleton.

As the organic compound having an electron-transport property that can be used for the electron-transport layer 114, any of the aforementioned organic compounds that can be used as the organic compound having an electron-transport property in the light-emitting layer 113 can be used. Among the above organic compounds, the organic compound that has a heteroaromatic ring having a diazine skeleton, the organic compound that has a heteroaromatic ring having a pyridine skeleton, and the organic compound that has a heteroaromatic ring having a triazine skeleton are especially preferable because of having high reliability. In particular, the organic compound that has a heteroaromatic ring having a diazine (pyrimidine or pyrazine) skeleton and the organic compound that has a heteroaromatic ring having a triazine skeleton have a high electron-transport property to contribute to a reduction in driving voltage. In particular, an organic compound having a phenanthroline skeleton such as mTpPPhen, PnNPhen, or mPPhen2P is preferable, and an organic compound having a phenanthroline dimer structure such as mPPhen2P is further preferable because of high stability.

The organic compound represented by General Formula (G3) in Embodiment 1 also has a π-electron deficient heteroaromatic ring skeleton and thus can be suitably used as the material having an electron-transport property.

Note that the electron-transport layer 114 may have a stacked-layer structure. A layer in the stacked-layer structure of the electron-transport layer 114, which is in contact with the light-emitting layer 113, may function as a hole-blocking layer. In the case where the electron-transport layer in contact with the light-emitting layer functions as a hole-blocking layer, the electron-transport layer is preferably formed using a material having a lower HOMO level than a material contained in the light-emitting layer 113 by greater than or equal to 0.5 eV.

A layer that contains a compound or a complex of an alkali metal or an alkaline earth metal such as 8-hydroxyquinolinato-lithium (abbreviation: Liq), 1,1′-pyridine-2,6-diyl-bis(1,3,4,6,7,8-hexahydro-2H-pyrimido[1,2-a]pyrimidine) (abbreviation: hpp2Py), the organic compound represented by General Formula (G3) in Embodiment 1, or the like may be provided as the electron-injection layer 115. As the electron-injection layer 115, an alkali metal, an alkaline earth metal, or a compound thereof may be contained in a layer formed using a substance having an electron-transport property.

Instead of the electron-injection layer 115, a charge-generation layer 116 may be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a second layer 117 that is a p-type layer. The second layer 117 is preferably formed using any of the composite materials given above as examples of materials that can be used for the hole-injection layer 111. The second layer 117 may be formed by stacking a film containing the above-described acceptor material as a material included in the composite material and a film containing a hole-transport material. When a potential is applied to the second layer 117, electrons are injected into the electron-transport layer 114 and holes are injected into the cathode; thus, the light-emitting device operates. Since the organic compound of one embodiment of the present invention is an organic compound capable of forming a film with a low refractive index, using the organic compound for the second layer 117 enables the light-emitting device to have high external quantum efficiency.

Note that the charge-generation layer 116 preferably includes one or both of a first layer 119 that is an electron-injection buffer layer and a third layer 118 that is an electron-relay layer in addition to the second layer 117 that is a p-type layer.

The first layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the first layer 119 contains a substance having an electron-transport property and a donor substance, the donor substance can be an organic compound such as tetrathianaphthacene (abbreviation: TTN), nickelocene, or decamethylnickelocene, as well as an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

In the case where the first layer 119 contains the substance having an electron-transport property and the donor substance, the first layer 119 preferably further contains the organic compound of one embodiment of the present invention that is represented by General Formula (G3) to inhibit an increase in driving voltage at the time of processing the EL layer by a photolithography method.

The third layer 118 contains at least the substance having an electron-transport property and has a function of preventing an interaction between the first layer 119 and the second layer 117 and smoothly transferring electrons. The LUMO level of the substance having an electron-transport property that is contained in the third layer 118 is preferably between the LUMO level of the acceptor substance in the second layer 117 and the LUMO level of a substance in a layer of the electron-transport layer 114 that is in contact with the charge-generation layer 116. As a specific value of the energy level, the LUMO level of the substance having an electron-transport property in the third layer 118 is preferably higher than or equal to −5.0 eV, further preferably higher than or equal to −5.0 eV and lower than or equal to −3.0 eV. Note that the substance having an electron-transport property that is used in the third layer 118 is preferably a phthalocyanine-based material or a metal complex having a metal-oxygen bond and an aromatic ligand.

The second electrode 102 includes the cathode. The second electrode 102 may have a stacked-layer structure, in which case a layer in contact with the organic compound layer 103 functions as the cathode. The cathode is preferably formed using a metal, an alloy, an electrically conductive compound, or a mixture thereof each having a low work function (specifically, lower than or equal to 3.8 eV), for example. Specific examples of such a cathode material include elements belonging to Group 1 or 2 of the periodic table, such as alkali metals (e.g., lithium (Li) or cesium (Cs)), magnesium (Mg), calcium (Ca), and strontium (Sr), alloys containing these elements (e.g., MgAg and AlLi), compounds containing these elements (e.g., lithium fluoride (LiF), cesium fluoride (CsF), and calcium fluoride (CaF₂)), rare earth metals such as europium (Eu) and ytterbium (Yb), and alloys containing these rare earth metals. However, when the electron-injection layer 115 or a thin film formed using any of the above materials having a low work function is provided between the second electrode 102 and the electron-transport layer, a variety of conductive materials such as Al, Ag, ITO, or indium oxide-tin oxide containing silicon or silicon oxide can be used for the cathode regardless of the work function.

When the second electrode 102 is formed using a material that transmits visible light, the light-emitting device can emit light from the second electrode 102 side.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different film formation methods may be used to form the electrodes or the layers described above.

Next, an embodiment of a light-emitting device with a structure in which a plurality of light-emitting units are stacked (this type of light-emitting device is also referred to as a stacked or tandem device) is described with reference to FIG. 1C. This light-emitting device includes a plurality of light-emitting units between an anode and a cathode. One light-emitting unit has substantially the same structure as the organic compound layer 103 illustrated in FIG. 1A. In other words, the light-emitting device illustrated in FIG. 1C includes a plurality of light-emitting units, and the light-emitting device illustrated in FIG. 1A or 1B includes a single light-emitting unit.

In FIG. 1C, a first light-emitting unit 511 and a second light-emitting unit 512 are stacked between a first electrode 501 and a second electrode 502, and an intermediate layer 513 is provided between the first light-emitting unit 511 and the second light-emitting unit 512. The first electrode 501 and the second electrode 502 correspond, respectively, to the first electrode 101 and the second electrode 102 illustrated in FIG. 1A, and can be formed using the materials given in the description for FIG. 1A. Furthermore, the first light-emitting unit 511 and the second light-emitting unit 512 may have the same structure or different structures.

The intermediate layer 513 has a function of injecting electrons into one of the light-emitting units and injecting holes into the other of the light-emitting units when voltage is applied between the first electrode 501 and the second electrode 502. That is, in FIG. 1C, the intermediate layer 513 injects electrons into the first light-emitting unit 511 and holes into the second light-emitting unit 512 when voltage is applied such that the potential of the anode becomes higher than the potential of the cathode.

The intermediate layer 513 preferably has a structure similar to that of the charge-generation layer 116 described with reference to FIG. 1B. A composite material of an organic compound and a metal oxide enables low-voltage driving and low-current driving because of having an excellent carrier-injection property and an excellent carrier-transport property.

In particular, the first layer 119 in the intermediate layer 513 preferably contains the organic compound represented by General Formula (G3) in Embodiment 1. When the first layer 119 contains the organic compound represented by General Formula (G3) in Embodiment 1, the light-emitting device can have a favorable electron-injection property and a low driving voltage.

The first layer 119 containing the organic compound represented by General Formula (G3) in Embodiment 1 can have an improved donor property of a metal or a metal compound owing to coordination of the organic compound represented by General Formula (G3) to the metal or the metal compound. This can inhibit impairment of the function of the first layer 119 in the intermediate layer 513 even when an organic compound layer 503 is exposed to an air atmosphere; thus, an increase in driving voltage can be inhibited and a light-emitting device with favorable characteristics can be provided.

In other words, a tandem light-emitting device including the intermediate layer 513 including the first layer 119 containing the organic compound represented by General Formula (G3) in Embodiment 1 and a metal or a metal compound can have a low driving voltage and favorable characteristics.

Furthermore, a tandem light-emitting device including the intermediate layer 513 including the first layer 119 containing the organic compound represented by General Formula (G3) in Embodiment 1, a metal or a metal compound, and an organic compound having an electron-transport property can have favorable characteristics with no significant increase in driving voltage even after processing by a photolithography method including an air exposure step.

In the case where the anode-side surface of a light-emitting unit is in contact with the intermediate layer 513, the intermediate layer 513 can also function as a hole-injection layer of the light-emitting unit; therefore, a hole-injection layer is not necessarily provided in the light-emitting unit.

In the case where the first layer 119 is provided in the intermediate layer 513, the first layer 119 functions as the electron-injection layer in the light-emitting unit on the anode side; thus, an electron-injection layer is not necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. With a plurality of light-emitting units partitioned by the intermediate layer 513 between a pair of electrodes as in the light-emitting device of this embodiment, it is possible to provide a long-life element that can emit light with high luminance at a low current density. A light-emitting apparatus that can be driven at a low voltage and has low power consumption can also be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as a whole.

The above-described layers and electrodes such as the organic compound layer 103, the first light-emitting unit 511, the second light-emitting unit 512, and the intermediate layer 513 can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the above components.

This embodiment can be combined as appropriate with the other embodiments or the examples. In this specification, in the case where a plurality of structure examples are shown in one embodiment, the structure examples can be combined as appropriate.

### Embodiment 3

Described in this embodiment is an example in which the light-emitting device of one embodiment of the present invention is used as a display element of a display device. Note that although a light-emitting device shown in this embodiment is formed by a photolithography method, the light-emitting device may be formed by a method using a fine metal mask or the like.

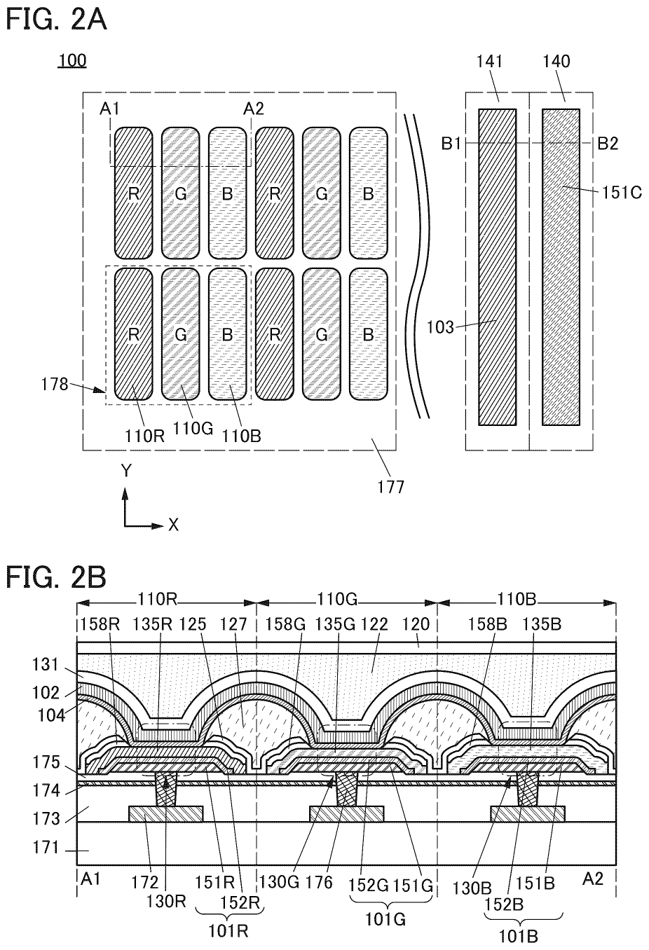

As illustrated in FIG. 2B, a plurality of light-emitting devices 130 are formed over an insulating layer 175 to constitute a display device.

The display device includes a pixel portion 177 in which a plurality of pixels 178 are arranged in a matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 2A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 2A illustrates an example where the region 141 and the connection portion 140 are positioned on the right side of the pixel portion 177, the positions of the region 141 and the connection portion 140 are not particularly limited. The number of the regions 141 and the number of the connection portions 140 can each be one or more.

FIG. 2B is an example of a cross-sectional view along the dashed-dotted line A1-A2 in FIG. 2A. As illustrated in FIG. 2B, the display device includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 are preferably provided between the adjacent light-emitting devices 130.

Although FIG. 2B illustrates cross sections of a plurality of the inorganic insulating layers 125 and a plurality of the insulating layers 127, the inorganic insulating layers 125 are preferably connected to each other and the insulating layers 127 are preferably connected to each other when the display device is seen from above. That is, the inorganic insulating layer 125 and the insulating layer 127 preferably include opening portions over first electrodes.

In FIG. 2B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light. It can be said that in FIG. 2B, the light-emitting devices 130R and 130G are adjacent light-emitting devices and the light-emitting devices 130G and 130B are adjacent light-emitting devices.

The display device of one embodiment of the present invention can be, for example, a top-emission display device where light is emitted in the direction opposite to a substrate over which light-emitting devices are formed. Note that the display device of one embodiment of the present invention may be of a bottom emission type.

The light-emitting device 130R emits red light (preferably emits phosphorescent light), and preferably has the structure shown in Embodiment 2. The light-emitting device 130R includes a first electrode (pixel electrode) including a conductive layer 151R and a conductive layer 152R, a first layer 135R over the first electrode, the common layer 104 over the first layer 135R, and the second electrode 102 (common electrode) over the common layer 104. The common layer 104 is preferably an electron-injection layer or a stack of an electron-transport layer and an electron-injection layer.

The light-emitting device 130G emits green light (preferably emits phosphorescent light), and preferably has the structure shown in Embodiment 2. The light-emitting device 130G includes a first electrode (pixel electrode) including a conductive layer 151G and a conductive layer 152G, a first layer 135G over the first electrode, the common layer 104 over the first layer 135G, and the second electrode 102 (common electrode) over the common layer 104. The common layer 104 is preferably an electron-injection layer or a stack of an electron-transport layer and an electron-injection layer.

The light-emitting device 130B emits blue light (preferably emits fluorescent light), and preferably has the structure shown in Embodiment 2. The light-emitting device 130B includes a first electrode (pixel electrode) including a conductive layer 151B and a conductive layer 152B, a first layer 135B over the first electrode, the common layer 104 over the first layer 135B, and the second electrode 102 (common electrode) over the common layer 104. The common layer 104 is preferably an electron-injection layer or a stack of an electron-transport layer and an electron-injection layer. Note that the stack of the first layer 135 and the common layer 104 corresponds to the organic compound layer 103 in FIG. 1A or the like.

In the case where the common layer 104 is not provided, the first layer 135 corresponds to the organic compound layer 103.

In the light-emitting device, one of the pixel electrode (first electrode) and the common electrode (second electrode) functions as an anode and the other functions as a cathode. In this embodiment, description is made on the assumption that the pixel electrode functions as the anode and the common electrode functions as the cathode unless otherwise specified.

The first layers 135R, 135G, and 135B are island-shaped layers that are independent of each other on a light-emitting device basis or on an emission color basis. It is preferable that the first layers 135R, 135G, and 135B not overlap with one another. The first layers included in the plurality of light-emitting devices 130 formed in the light-emitting apparatus, such as the first layers 135R, 135G, and 135B, are collectively referred to as a first layer group 135A in some cases. Providing the island-shaped first layer group 135A in the light-emitting devices 130 can inhibit leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped first layer group 135A is formed by forming an EL film for each emission color and processing the EL film by a photolithography technique.

The first layer 135 is preferably provided to cover the top surface and the side surface of the first electrode 101 (pixel electrode) of the light-emitting device 130. In this case, the aperture ratio of the display device can be easily increased as compared to the structure where an end portion of the first layer 135 is positioned inside an end portion of the pixel electrode. Covering the side surface of the pixel electrode of the light-emitting device 130 with the first layer 135 can inhibit the first electrode 101 from being in contact with the second electrode 102; hence, a short circuit of the light-emitting device 130 can be inhibited.

In the display device of one embodiment of the present invention, the first electrode 101 (pixel electrode) of the light-emitting device preferably has a stacked-layer structure. For example, in the example illustrated in FIG. 2B, the first electrode 101 of the light-emitting device 130 has a stacked-layer structure of the conductive layer 151 provided on the insulating layer 171 side and the conductive layer 152 provided on the organic compound layer side.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, an indium tin oxide containing silicon can be suitably used for the conductive layer 152 because of having a work function of higher than or equal to 4.0 eV, for example.

The conductive layer 151 and the conductive layer 152 may each be a stack of a plurality of layers containing different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

Note that the conductive layer 151 preferably has a tapered end portion. Specifically, the conductive layer 151 preferably has a tapered end portion with a taper angle of less than 90°. In that case, the conductive layer 152 provided along the side surface of the conductive layer 151 also has a tapered shape. When the side surface of the conductive layer 152 has a tapered shape, coverage with the first layer 135 provided along the side surface of the conductive layer 152 can be improved.

This embodiment can be combined as appropriate with the other embodiments or the examples. In this specification, in the case where a plurality of structure examples are shown in one embodiment, the structure examples can be combined as appropriate.

### Embodiment 4

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### [Display Module]

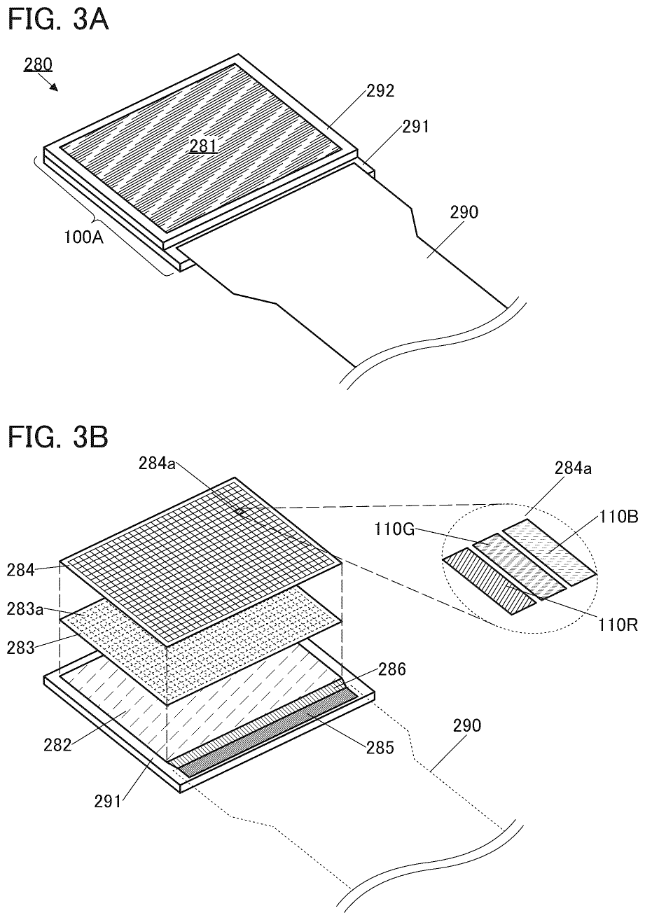

FIG. 3A is a perspective view of a display module 280. The display module 280 includes a display device 100A and an FPC 290. Note that the display device included in the display module 280 is not limited to the display device 100A and may be any of display devices 100B to 100E described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 3B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 3B. The pixels 284a can employ any of the structures described in the above embodiments. FIG. 3B illustrates an example where the pixel 284a has a structure similar to that of the pixel 178 illustrated in FIG. 2A.

The pixel circuit portion 283 includes a plurality of pixel circuits 283a arranged periodically.

One pixel circuit 283a is a circuit that controls driving of a plurality of elements included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion.

### [Display Device 100A]

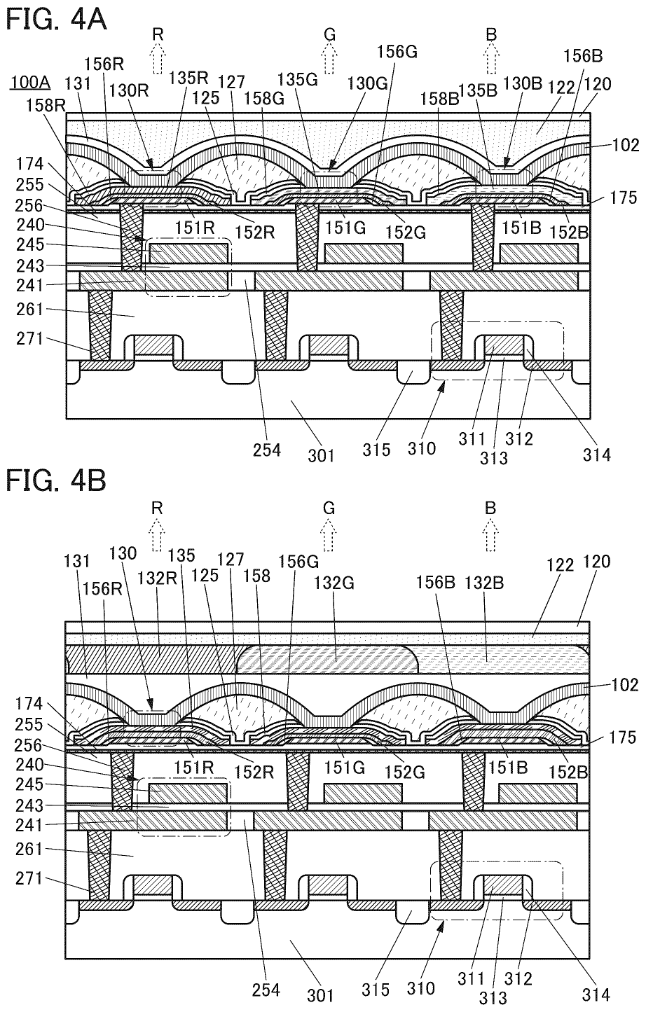

The display device 100A illustrated in FIG. 4A includes a substrate 301, the light-emitting devices 130R, 130G, and 130B, a capacitor 240, and a transistor 310.

The substrate 301 corresponds to the substrate 291 in FIGS. 3A and 3B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

The insulating layer 156R is provided to include a region overlapping with the side surface of the conductive layer 151R. The insulating layer 156G is provided to include a region overlapping with the side surface of the conductive layer 151G. The insulating layer 156B is provided to include a region overlapping with the side surface of the conductive layer 151B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. A sacrificial layer 158R is positioned over the first layer 135R. A sacrificial layer 158G is positioned over the first layer 135G. A sacrificial layer 158B is positioned over the first layer 135B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 3 can be referred to for the details of the light-emitting device 130 and the components thereover up to the substrate 120. The substrate 120 corresponds to the substrate 292 in FIG. 3A.

FIG. 4B illustrates a variation example of the display device 100A illustrated in FIG. 4A. The display device illustrated in FIG. 4B includes a coloring layer 132R, a coloring layer 132G, and a coloring layer 132B, and each of the light-emitting devices 130 includes a region overlapping with one of the coloring layers 132R, 132G, and 132B. In the display device illustrated in FIG. 4B, the light-emitting device 130 can emit white light, for example. The coloring layer 132R, the coloring layer 132G, and the coloring layer 132B can transmit red light, green light, and blue light, respectively, for example.

### [Display Device 100B]

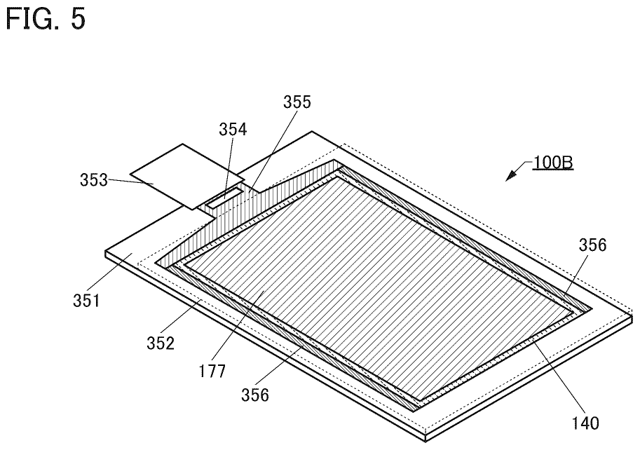

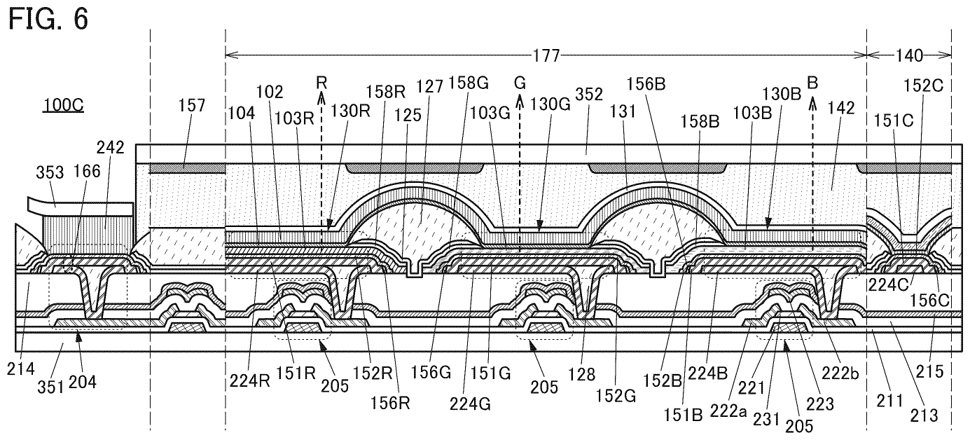

FIG. 5 is a perspective view of the display device 100B, and FIG. 6 is a cross-sectional view of the display device 100C.

In the display device 100B, a substrate 352 and a substrate 351 are bonded to each other. In FIG. 5, the substrate 352 is denoted by a dashed line.

The display device 100B includes the pixel portion 177, the connection portion 140, a circuit 356, a wiring 355, and the like. FIG. 5 illustrates an example where an IC 354 and an FPC 353 are mounted on the display device 100B. Thus, the structure illustrated in FIG. 5 can be regarded as a display module including the display device 100B, the integrated circuit (IC), and the FPC. Here, a display device in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 5 illustrates an example where the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

FIG. 6 illustrates an example of cross sections of part of a region including the FPC 353, part of the circuit 356, part of the pixel portion 177, part of the connection portion 140, and part of a region including an end portion of the display device 100B.

### [Display Device 100C]

The display device 100C illustrated in FIG. 6 includes a transistor 201, a transistor 205, the light-emitting device 130R that emits red light, the light-emitting device 130G that emits green light, the light-emitting device 130B that emits blue light, and the like between the substrate 351 and the substrate 352.

Embodiment 2 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through an opening provided in an insulating layer 214. An end portion of the conductive layer 151R is positioned outside an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G, and the insulating layer 156G in the light-emitting device 130G are not described in detail because they are respectively similar to the conductive layers 224R, 151R, and 152R, and the insulating layer 156R in the light-emitting device 130R; the same applies to the conductive layers 224B, 151B, and 152B, and the insulating layer 156B in the light-emitting device 130B.

The conductive layers 224R, 224G, and 224B each have a depressed portion covering the opening provided in the insulating layer 214. A layer 128 is embedded in the depressed portion.

The layer 128 has a function of filling the depressed portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 6, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap with the light-emitting device. Furthermore, the space may be filled with a resin other than the frame-like adhesive layer 142.

FIG. 6 illustrates an example where the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and a conductive layer 152C obtained by processing the same conductive film as the conductive layers 152R, 152G, and 152B. In the example illustrated in FIG. 6, an insulating layer 156C is provided to include a region overlapping with the side surface of the conductive layer 151C.

The display device 100B has a top-emission structure. Light from the light-emitting device is emitted toward the substrate 352. For the substrate 352, a material with a high visible-light-transmitting property is preferably used. In the case where the light-emitting device emits infrared or near-infrared light, a material having a high transmitting property with respect to infrared or near-infrared light is preferably used. The first electrode (pixel electrode) contains a material that reflects visible light, and the second electrode (counter electrode) contains a material that transmits visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used as each of the insulating layers 211, 213, and 215.

An organic insulating layer is suitable as the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 not overlapping with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 100D]

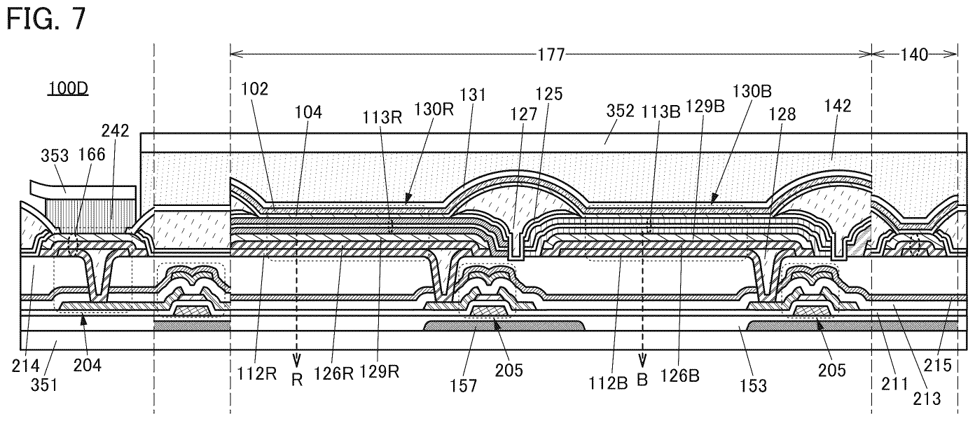

The display device 100D illustrated in FIG. 7 differs from the display device 100C illustrated in FIG. 6 mainly in having a bottom-emission structure.

Light from the light-emitting device is emitted toward the substrate 351. For the substrate 351, a material with a high visible-light-transmitting property is preferably used. By contrast, there is no limitation on the light-transmitting property of a material used for the substrate 352.

A light-blocking layer is preferably formed between the substrate 351 and the transistor 201 and between the substrate 351 and the transistor 205. FIG. 7 illustrates an example where the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material with a high visible-light-transmitting property is used for each of the conductive layers 112R, 112B, 126R, 126B, 129R, and 129B. A material that reflects visible light is preferably used for the second electrode.

Although not illustrated in FIG. 7, the light-emitting device 130G is also provided.

Although FIG. 7 and the like illustrate an example where the top surface of the layer 128 includes a flat portion, the shape of the layer 128 is not particularly limited.

### [Display Device 100E]

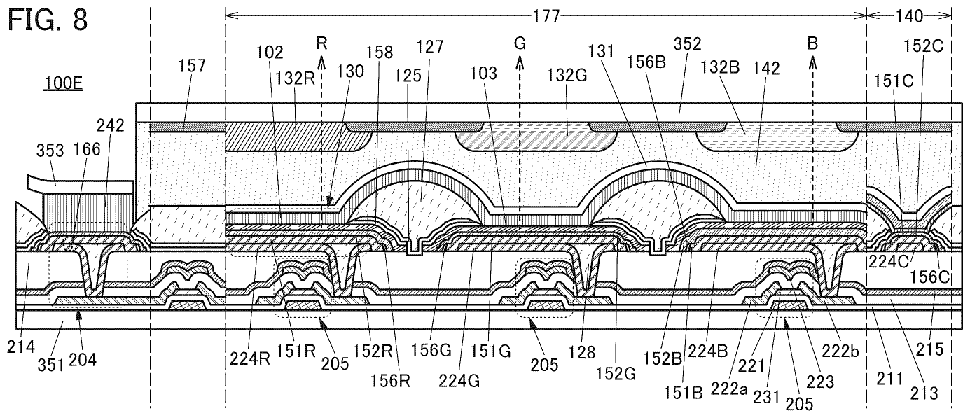

The display device 100E illustrated in FIG. 8 is a variation example of the display device 100C illustrated in FIG. 6 and differs from the display device 100C mainly in including the coloring layers 132R, 132G, and 132B.

In the display device 100E, the light-emitting device 130 includes a region overlapping with one of the coloring layers 132R, 132G, and 132B. The coloring layers 132R, 132G, and 132B can be provided on a surface of the substrate 352 on the substrate 351 side. End portions of the coloring layers 132R, 132G, and 132B can overlap with the light-blocking layer 157.

In the display device 100E, the light-emitting device 130 can emit white light, for example. The coloring layer 132R, the coloring layer 132G, and the coloring layer 132B can transmit red light, green light, and blue light, respectively, for example. Note that in the display device 100E, the coloring layers 132R, 132G, and 132B may be provided between the protective layer 131 and the adhesive layer 142.

Although FIGS. 6 and 8 and the like illustrate an example where the top surface of the layer 128 includes a flat portion, the shape of the layer 128 is not particularly limited.

This embodiment can be combined as appropriate with the other embodiments or the examples. In this specification, in the case where a plurality of structure examples are shown in one embodiment, the structure examples can be combined as appropriate.

### Embodiment 5

In this embodiment, electronic appliances of embodiments of the present invention will be described.

Electronic appliances in this embodiment each include the display device of one embodiment of the present invention in a display portion. The display device of one embodiment of the present invention has low power consumption. Thus, the display device of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the display device of one embodiment of the present invention has low power consumption, and thus can be suitably used for a relatively small electronic appliance. Examples of such an electronic appliance include watch-type and bracelet-type information terminals (wearable devices) and wearable devices capable of being worn on a head, such as a VR device like a head-mounted display, a glasses-type AR device, and an MR device.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

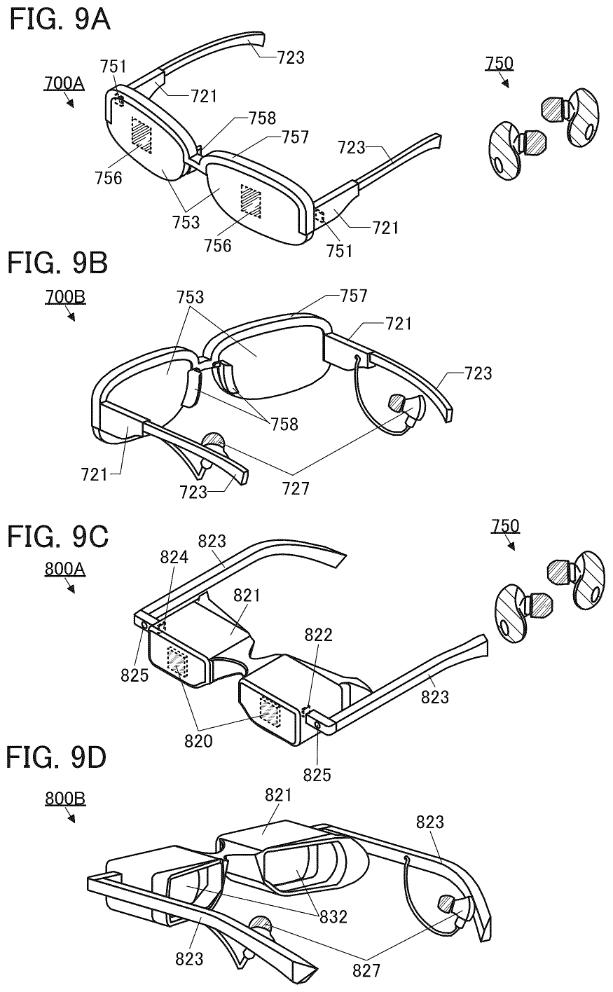

Examples of wearable devices capable of being worn on a head are described with reference to FIGS. 9A to 9D.

An electronic appliance 700A illustrated in FIG. 9A and an electronic appliance 700B illustrated in FIG. 9B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, the electronic appliance can have low power consumption and be driven for a long time.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

An electronic appliance 800A illustrated in FIG. 9C and an electronic appliance 800B illustrated in FIG. 9D each include a pair of display portions 820, a housing 821, a communication portion 822, a pair of wearing portions 823, a control portion 824, a pair of image capturing portions 825, and a pair of lenses 832.

The display device of one embodiment of the present invention can be used in the display portions 820. Thus, the electronic appliance can have low power consumption and be driven for a long time.

The display portions 820 are positioned inside the housing 821 so as to be seen through the lenses 832. When the pair of display portions 820 display different images, three-dimensional display using parallax can be performed.

The electronic appliances 800A and 800B preferably include a mechanism for adjusting the lateral positions of the lenses 832 and the display portions 820 so that the lenses 832 and the display portions 820 are positioned optimally in accordance with the positions of the user's eyes.

The electronic appliance 800A or the electronic appliance 800B can be mounted on the user's head with the wearing portions 823.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to support a plurality of fields of view, such as a telescope field of view and a wide field of view.

The electronic appliance 800A may include a vibration mechanism that functions as bone-conduction earphones.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750.

The electronic appliance may include an earphone portion. The electronic appliance 700B illustrated in FIG. 9B includes earphone portions 727. Part of a wiring that connects the earphone portion 727 and the control portion may be positioned inside the housing 721 or the wearing portion 723.

Similarly, the electronic appliance 800B illustrated in FIG. 9D includes earphone portions 827. For example, the earphone portion 827 can be connected to the control portion 824 by wire.

As described above, both the glasses-type device (e.g., the electronic appliances 700A and 700B) and the goggles-type device (e.g., the electronic appliances 800A and 800B) are preferable as the electronic appliance of one embodiment of the present invention.

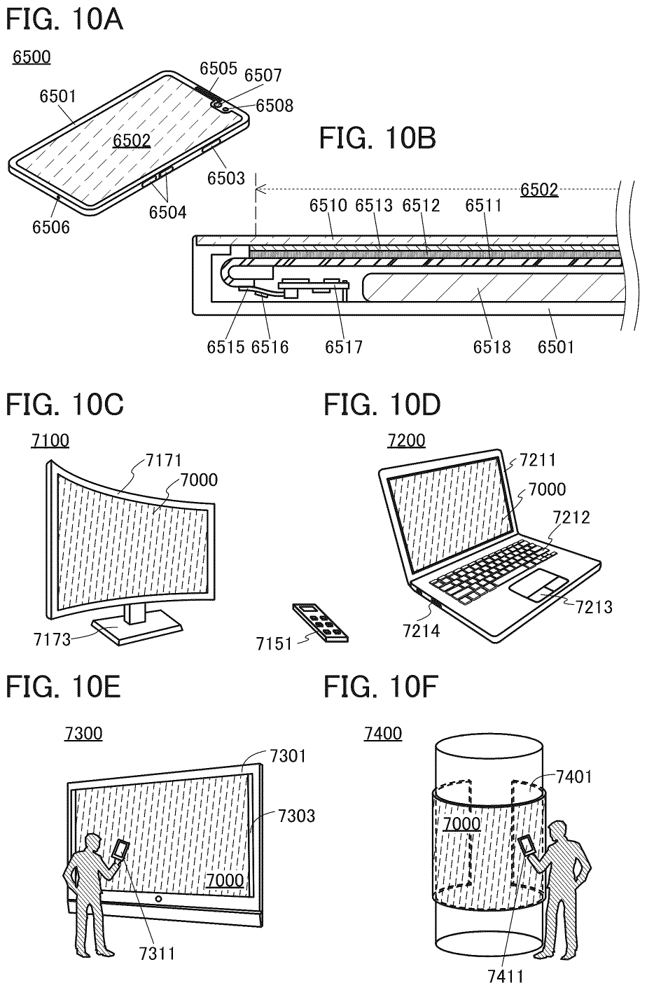

An electronic appliance 6500 illustrated in FIG. 10A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, the electronic appliance can have low power consumption and be driven for a long time.

FIG. 10B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with a bonding layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, an extremely lightweight electronic appliance can be achieved. Since the display panel 6511 is extremely thin, the battery 6518 with high capacity can be mounted without an increase in the thickness of the electronic appliance. Moreover, part of the display panel 6511 is folded back so that a connection portion with the FPC 6515 is provided on the back side of the pixel portion, whereby an electronic appliance with a narrow bezel can be achieved.

FIG. 10C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, the electronic appliance can have low power consumption and be driven for a long time.

Operation of the television device 7100 illustrated in FIG. 10C can be performed with an operation switch provided in the housing 7171 and a separate remote controller 7151.

FIG. 10D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, the electronic appliance can have low power consumption and be driven for a long time.

FIGS. 10E and 10F illustrate examples of digital signage.

Digital signage 7300 illustrated in FIG. 10E includes a housing 7301, the display portion 7000, a speaker 7303, and the like. The digital signage 7300 can also include an LED lamp, an operation key (including a power switch or an operation switch), a connection terminal, a variety of sensors, a microphone, and the like.

FIG. 10F illustrates digital signage 7400 attached to a cylindrical pillar 7401. The digital signage 7400 includes the display portion 7000 provided along a curved surface of the pillar 7401.

In FIGS. 10E and 10F, the display device of one embodiment of the present invention can be used in the display portion 7000. Thus, a highly reliable electronic appliance is obtained.

A larger area of the display portion 7000 can increase the amount of information that can be provided at a time. The larger display portion 7000 attracts more attention, so that the effectiveness of the advertisement can be increased, for example.

As illustrated in FIGS. 10E and 10F, it is preferable that the digital signage 7300 or the digital signage 7400 can work with an information terminal 7311 or an information terminal 7411, such as a smartphone that a user has, through wireless communication.

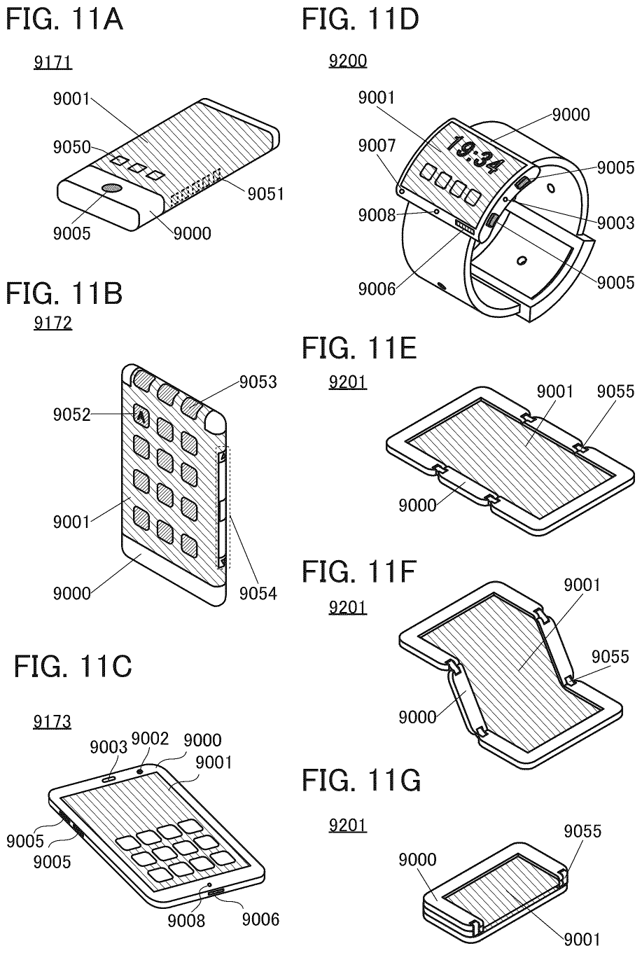

Electronic appliances illustrated in FIGS. 11A to 11G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 11A to 11G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with use of a variety of software (programs), a wireless communication function, and a function of reading out and processing a program or data stored in a recording medium.

The electronic appliances illustrated in FIGS. 11A to 11G are described in detail below.

FIG. 11A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 11A illustrates an example where three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 11B is a perspective view of a portable information terminal 9172. The portable information terminal 9172 has a function of displaying information on three or more surfaces of the display portion 9001. In the example illustrated here, information 9052, information 9053, and information 9054 are displayed on different surfaces. For example, the user of the portable information terminal 9172 can check the information 9053 displayed such that it can be seen from above the portable information terminal 9172, with the portable information terminal 9172 put in a breast pocket of his/her clothes.

FIG. 11C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 11D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 11E to 11G are perspective views of a foldable portable information terminal 9201. FIG. 11E is a perspective view illustrating the portable information terminal 9201 that is opened. FIG. 11G is a perspective view illustrating the portable information terminal 9201 that is folded. FIG. 11F is a perspective view illustrating the portable information terminal 9201 that is shifted from one of the states in FIGS. 11E and 11G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

Described in this synthesis example is a method for synthesizing 4-chloro-7-(2,3,3a,4,5,6,7,7a-octahydro-1H-isoindol-2-yl)-1,10-phenanthroline (abbreviation: 4Cl7HidPhen), which is the organic compound of the present invention that is represented by Structural Formula (259) in Embodiment 1. The structure of 4Cl7HidPhen is shown below.

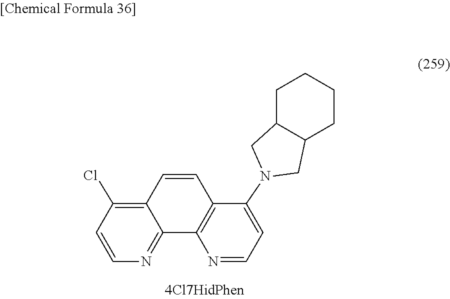 [SMILES: Clc1ccnc2c1ccc1c(N3CC4CCCCC4C3)ccnc12]

Into a 100-mL three-neck flask were put 2.9 g (12 mmol) of 4,7-dichloro-1,10-phenanthroline, 2.8 mL (24 mmol) of 2,3,3a,4,5,6,7,7a-octahydro-1H-isoindole, 4.9 g (35 mmol) of potassium carbonate (abbreviation: K₂CO₃), and 24 mL of N-methylpyrrolidone (abbreviation: NMP). The mixture was stirred at 80° C. for 5 hours. After the mixture was air-cooled to room temperature, 50 mL of water and 50 mL of chloroform were added to the flask, and the mixture was separated using a separating funnel. Extraction was performed three times using chloroform, and the organic layer was washed with pure water once. The organic layer was gravity-filtered, and the filtrate was concentrated to give a solid. A small amount of acetone was added to the solid to give a suspension of a target substance. The suspension was subjected to suction filtration to give 3.6 g of a target pale yellow solid (in a yield of 92%). Synthesis Scheme (s1-1) is shown below.

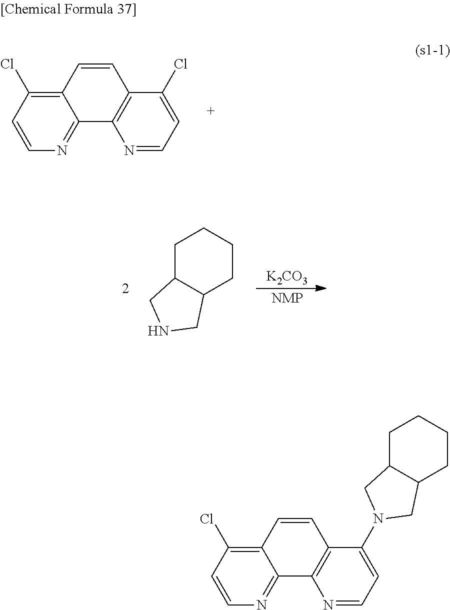

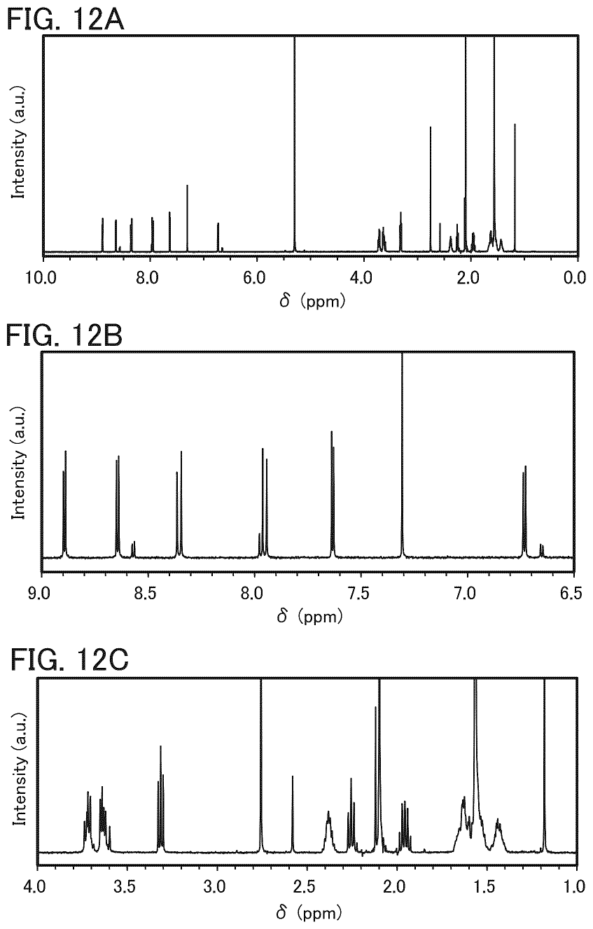

FIGS. 12A to 12C show the ¹H NMR measurement result of the pale yellow solid obtained. FIG. 12B is an enlarged graph of FIG. 12A in the range from 6.5 ppm to 9.0 ppm, and FIG. 12C is an enlarged graph of FIG. 12A in the range from 1 ppm to 4.0 ppm. Results of ¹H NMR measurement are shown below.

¹H NMR (CD₂Cl₂, 500 MHz): δ=8.89 (1H, d, J=5.0 Hz), 8.64 (1H, d, J=5.5 Hz), 8.35 (1H, d, J=10 Hz), 7.95 (1H, d, J=10 Hz), 7.63 (1H, d, J=4.5 Hz), 6.73 (1H, d, J=5.0 Hz), 3.75-3.60 (4H, m), 2.41-2.34 (2H, m), 1.70-1.38 (8H, m).

The molecular weight of the pale yellow solid obtained was measured by liquid chromatography/mass spectrometry analysis (abbreviation: LC/MS analysis).

In the LC/MS analysis, liquid chromatography (LC) separation was carried out with ACQUITY UPLC (Waters Corporation), and mass spectrometry (MS) analysis was carried out with Xevo G2 Tof MS (Waters Corporation). ACQUITY UPLC BEH C8 (2.1×100 mm, 1.7 μm) was used as a column for the LC separation, and the column temperature was set to 40° C. Acetonitrile was used for Mobile Phase A, and a 0.1% aqueous solution of formic acid was used for Mobile Phase B. As a sample, 2.0 mg of 4Cl7HidPhen was put in a sample bottle, to which 1.0 mL of dichloromethane was added with a micropipette, and dissolved to give 1.0 mL of the solution. To the solution, 9.0 mL of acetonitrile was added with a micropipette to adjust the concentration of 4Cl7HidPhen to 200 ppm. The injection amount of the sample was 5.0 μL.

In the LC separation, a gradient method in which the composition of mobile phases is changed was employed. The ratio of Mobile Phase A to Mobile Phase B was set at 30:70 for 0 to 1 minute after the start of the measurement, and then the composition was changed such that the ratio of Mobile Phase A to Mobile Phase B after 10 minutes from the start of the measurement was 95:5. The composition was changed linearly.

In the MS analysis, ionization was carried out by an electrospray ionization (ESI) method. Capillary voltage and sample cone voltage were set to 3.01075 kV and 30 V, respectively. Detection was carried out in a positive mode. The mass range for the measurement was m/z=100 to 1200.

As a result of the LC/MS analysis, a peak indicating m/z 338 was observed while the mass of 4Cl7HidPhen was calculated to be 337. This is probably a peak of a proton adduct of 4Cl7HidPhen.

The ¹H NMR and LC/MS analysis results indicate that 4Cl7HidPhen was obtained in this synthesis example.

Here, the results of performing a synthesis similar to that in this example by a synthesis method disclosed in Non-Patent Document 1 will be described. In the synthesis method disclosed in Non-Patent Document 1, diisopropylethylamine is used as a base.

Into a standard glass reaction vessel with a diameter of 24 mm were put 1.0 g (4.0 mmol) of 4,7-dichloro-1,10-phenanthroline, 1.0 g (8.1 mmol) of 2,3,3a,4,5,6,7,7a-octahydro-1H-isoindole, 1.6 g (12 mmol) of diisopropylethylamine, and 8 mL of N-methylpyrrolidone (abbreviation: NMP). The mixture was stirred at 80° C. for 5 hours. After the mixture was air-cooled to room temperature, the solid precipitated in the reaction vessel was collected by filtration to give 0.13 g of a yellow solid. Synthesis Scheme (s1-2) of this synthesis example is shown below.

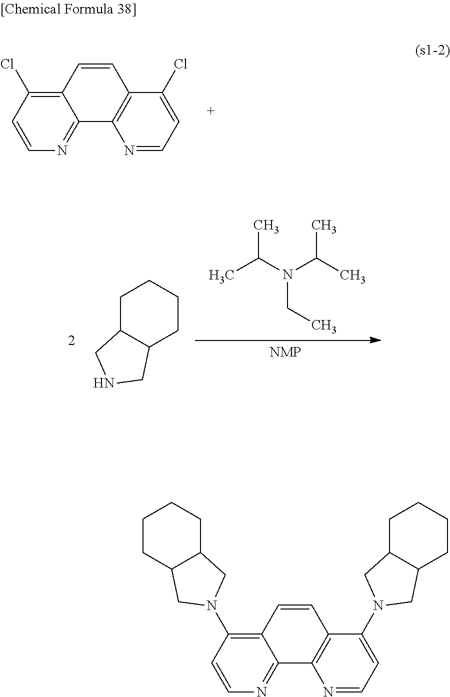

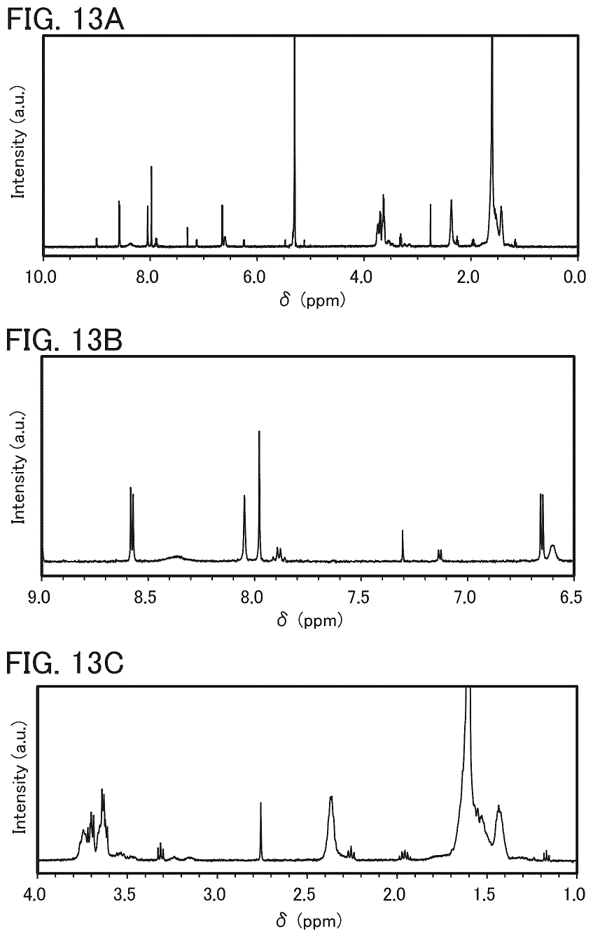

FIGS. 13A to 13C show the ¹H NMR measurement result of the yellow solid obtained. FIG. 13B is an enlarged graph of FIG. 13A in the range from 6.5 ppm to 9.0 ppm, and FIG. 13C is an enlarged graph of FIG. 13A in the range from 1 ppm to 4.0 ppm. Results of ¹H NMR measurement are shown below.

¹H NMR (CD₂Cl₂, 500 MHz): δ=8.58 (2H, d, J=5.5 Hz), 7.98 (2H, s), 6.65 (2H, d, J=5.0), 3.80-3.60 (8H, m), 2.45-2.30 (4H, m), 1.85-1.35 (16H, m).

The LC analysis and mass number measurement of the yellow solid obtained were performed by LC/MS analysis. As a result, a peak indicating m/z 427 was mainly observed in a chromatogram obtained. The peak at m/z 427 is probably a peak of a proton adduct of 4,7-di(2,3,3a,4,5,6,7,7a-octahydro-1H-isoindol-2-yl)-1,10-phenanthroline (abbreviation: Hid2Phen) (calculated mass: 426), which is an organic compound in which two 2,3,3a,4,5,6,7,7a-octahydro-1H-isoindol-2-yl groups are bonded to 1,10-phenanthroline.

The ¹H NMR and LC/MS analysis results indicate that Hid2Phen was obtained by the synthesis method disclosed in Non-Patent Document 1. This synthesis method can selectively provide only 4,7-di(2,3,3a,4,5,6,7,7a-octahydro-1H-isoindol-2-yl)-1,10-phenanthroline (abbreviation: Hid2Phen). In the case where two or more symmetric functional groups in such a compound exhibit substantially the same reactivity or reaction selectivity, it is difficult to selectively react only one of the functional groups. As a method for improving reaction selectivity in a general coupling reaction or the like, the selectivity may be obtained in some cases with a devised ligand of a transition metal catalyst, but there are quite few examples of controlling reactivity using a simple classical nucleophilic substitution reaction as in the method of one embodiment of the present invention. This suggests that the present invention provides an excellent method for asymmetrically modifying symmetric functional groups of a compound.

The above results reveal that 4Cl7HidPhen, which is the target substance, can hardly be obtained in the case of using diisopropylethylamine as in the synthesis method disclosed in Non-Patent Document 1. Such a mere difference in the type of base used leads to difficulty in synthesizing an asymmetric 1,10-phenanthroline derivative, which means that the synthesis method of one embodiment of the present invention is a very effective synthesis method for obtaining an asymmetric 1,10-phenanthroline derivative.

### Example 2

### Synthesis Example 2

Described in this example is a method for synthesizing 4-chloro-7-(1-pyrrolidinyl)-1,10-phenanthroline (abbreviation: 4Cl7PrdPhen) represented by Structural Formula (200) in Embodiment 1. The structure of 4Cl7PrdPhen is shown below.

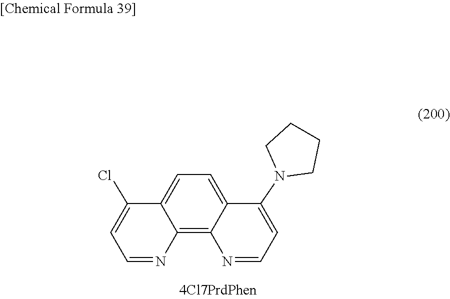 [SMILES: Clc1ccnc2c1ccc1c(N3CCCC3)ccnc12]

Into a 1000-mL three-neck flask were put 30 g (0.12 mol) of 4,7-dichloro-1,10-phenanthroline, 8.5 g (0.12 mol) of 1H-pyrrolidine, 50 g (0.36 mol) of potassium carbonate, and 0.24 L of 1-methyl-2-pyrrolidone (NMP). The mixture was stirred at 100° C. under a nitrogen stream for 5 hours. After the stirring, the mixture was cooled down to room temperature. An insoluble matter of the mixture was separated by suction filtration. The filtrate was subjected to extraction with dichloromethane. The extracted solution was concentrated to give an oily substance. Hexane was added to the oily substance, the mixture was stirred at 0° C., and the precipitated solid was collected by suction filtration. Ethyl acetate and hexane were added to the solid, irradiation with ultrasonic waves was performed, and the solid was collected by suction filtration to give a target light brown solid (19 g in a yield of 56%). The synthesis scheme of this synthesis example is shown in Formula (s2-1) below.

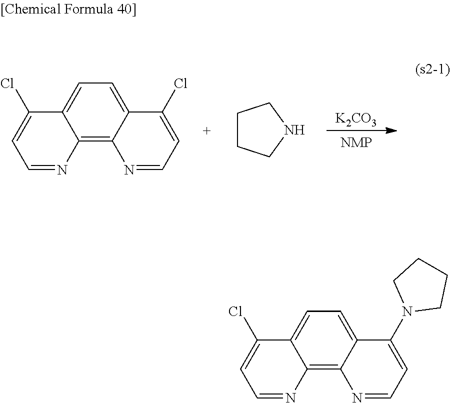

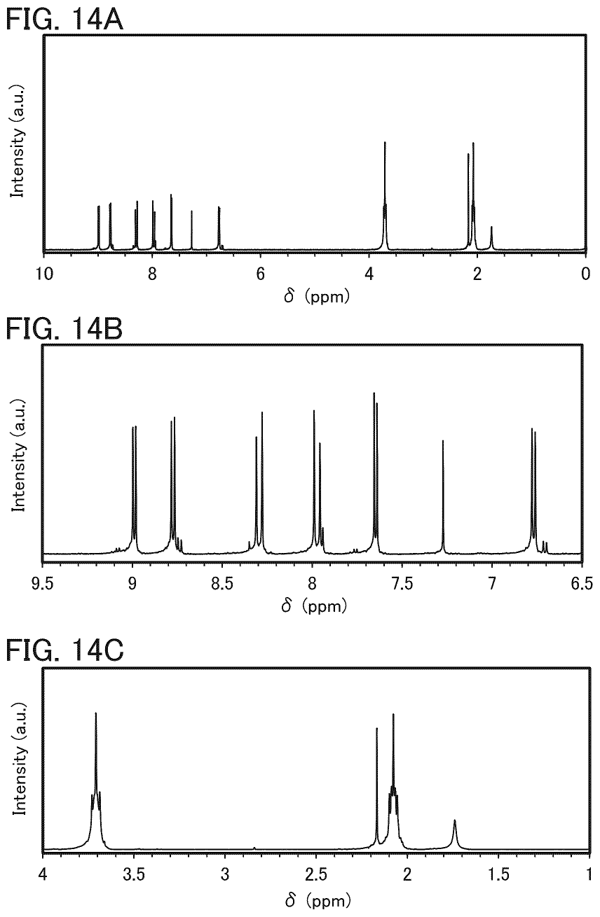

FIGS. 14A to 14C show the ¹H NMR spectrum of the light brown solid obtained. FIG. 14B is an enlarged graph of FIG. 14A in the range from 6.5 ppm to 9.5 ppm, and FIG. 14C is an enlarged graph of FIG. 14A in the range from 1 ppm to 4.0 ppm. Results of ¹H NMR measurement are shown below. The results reveal that 4Cl7PrdPhen was obtained.

¹H NMR (CDCl₃, 300 MHz): δ=8.99 (1H, d, J=4.8 Hz), 8.77 (1H, d, J=5.5 Hz), 8.29 (1H, d, J=9.5 Hz), 7.97 (1H, d, J=9.5 Hz), 7.65 (1H, d, J=4.8 Hz), 6.77 (1H, d, J=5.5 Hz), 3.73-3.69 (4H, m), 2.10-2.05 (4H, m).

The above results reveal that the synthesis method of one embodiment of the present invention does not impair selectivity even when the synthesis is scaled up.

### Example 3

### Synthesis Example 3

Described in this example is a method for synthesizing 4-bromo-7-(2,3,3a,4,5,6,7,7a-octahydro-1H-isoindol-2-yl)-1,10-phenanthroline (abbreviation: 4Br7HidPhen) represented by Structural Formula (202) in Embodiment 1. The structure of 4Br7HidPhen is shown below.

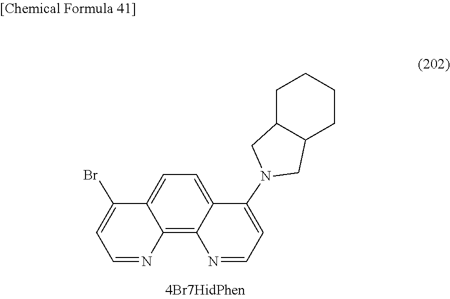 [SMILES: Brc1ccnc2c1ccc1c(N3CC4CCCCC4C3)ccnc12]

Into a 1000-mL three-neck flask were put 10 g (30 mmol) of 4,7-dibromo-1,10-phenanthroline, 3.7 g (30 mmol) of 2,3,3a,4,5,6,7,7a-octahydro-1H-isoindole, 12 g (87 mmol) of potassium carbonate, and 0.10 L of 1-methyl-2-pyrrolidone (NMP). The mixture was stirred at 100° C. under a nitrogen stream for 7 hours. After the stirring, the mixture was cooled down to room temperature. An insoluble matter of the mixture was separated by suction filtration. The filtrate was subjected to extraction with dichloromethane. The extracted solution was concentrated to give an oily substance. The oily substance was purified by silica gel column chromatography (developing solvent: ethyl acetate, chloroform, and methanol in this order). The resulting fraction was concentrated to give an oily substance. A small amount of chloroform and a small amount of ethyl acetate were added to the oily substance, irradiation with ultrasonic waves was performed, and the precipitated solid was collected by suction filtration to give a target yellow solid (7.6 g in a yield of 69%). The synthesis scheme of this synthesis example is shown in Formula (s3-1) below.

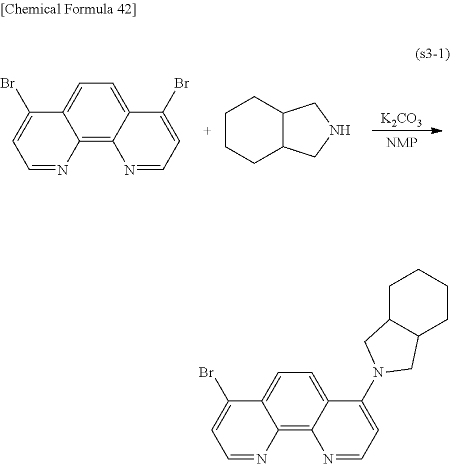

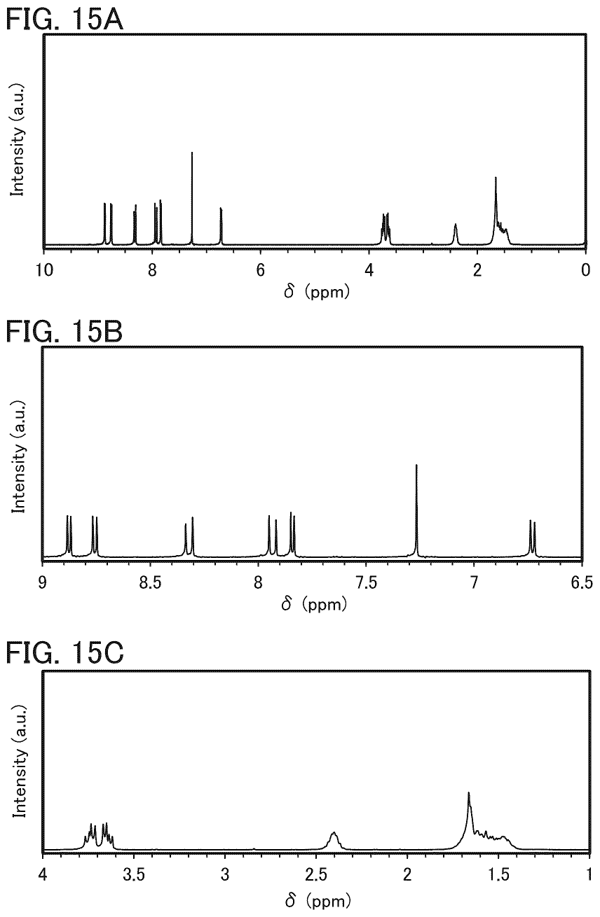

FIGS. 15A to 15C show the ¹H NMR spectrum of the yellow solid obtained. FIG. 15B is an enlarged graph of FIG. 15A in the range from 6.5 ppm to 9.0 ppm, and FIG. 15C is an enlarged graph of FIG. 15A in the range from 1 ppm to 4.0 ppm. Results of ¹H NMR measurement are shown below. The results reveal that 4Br7HidPhen was obtained.

¹H NMR (CDCl₃, 300 MHz): δ=8.88 (1H, d, J=4.8 Hz), 8.76 (1H, d, J=5.9 Hz), 8.32 (1H, d, J=9.5 Hz), 7.93 (1H, d, J=9.5 Hz), 7.84 (1H, d, J=4.8 Hz), 6.73 (1H, d, J=5.9 Hz), 3.77-3.62 (4H, m), 2.40 (2H, br), 1.62-1.45 (8H, m).

### Example 4

### Synthesis Example 4

Described in this example is a method for synthesizing 4-bromo-7-(1-pyrrolidinyl)-1,10-phenanthroline (abbreviation: 4Br7PrdPhen) represented by Structural Formula (201) in Embodiment 1. The structure of 4Br7PrdPhen is shown below.

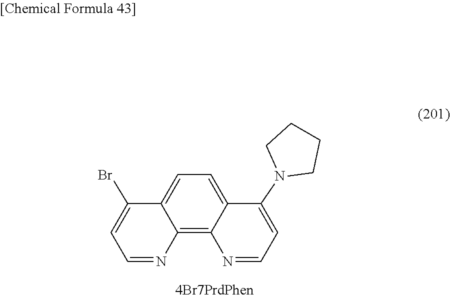 [SMILES: Brc1ccnc2c1ccc1c(N3CCCC3)ccnc12]

Into a 50-mL three-neck flask were put 1.5 g (4.4 mmol) of 4,7-dibromo-1,10-phenanthroline, 0.32 g (4.5 mmol) of 1H-pyrrolidine, 1.8 g (13 mmol) of potassium carbonate, and 10 mL of 1-methyl-2-pyrrolidone (NMP). The mixture was stirred at 100° C. under a nitrogen stream for 7 hours. After the stirring, the mixture was cooled down to room temperature. An insoluble matter of the mixture was separated by suction filtration. Water was added to the filtrate, and the mixture was subjected to extraction with dichloromethane. The extracted solution was concentrated to give an oily substance. The oily substance was purified by silica gel column chromatography (developing solvent: ethyl acetate, chloroform, and methanol in this order). The resulting fraction was concentrated to give an oily substance. A small amount of chloroform and a small amount of ethyl acetate were added to the oily substance, irradiation with ultrasonic waves was performed, and the precipitated solid was collected by suction filtration to give a target yellow solid (0.77 g in a yield of 51%). The synthesis scheme of this synthesis example is shown in Formula (s4-1) below.

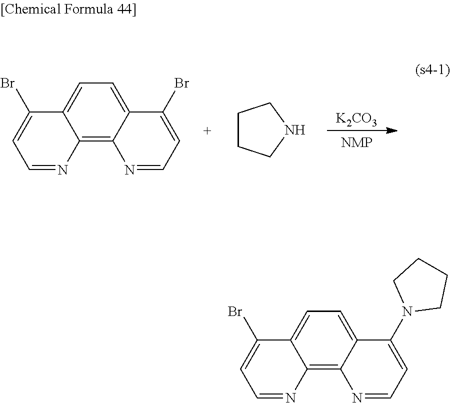

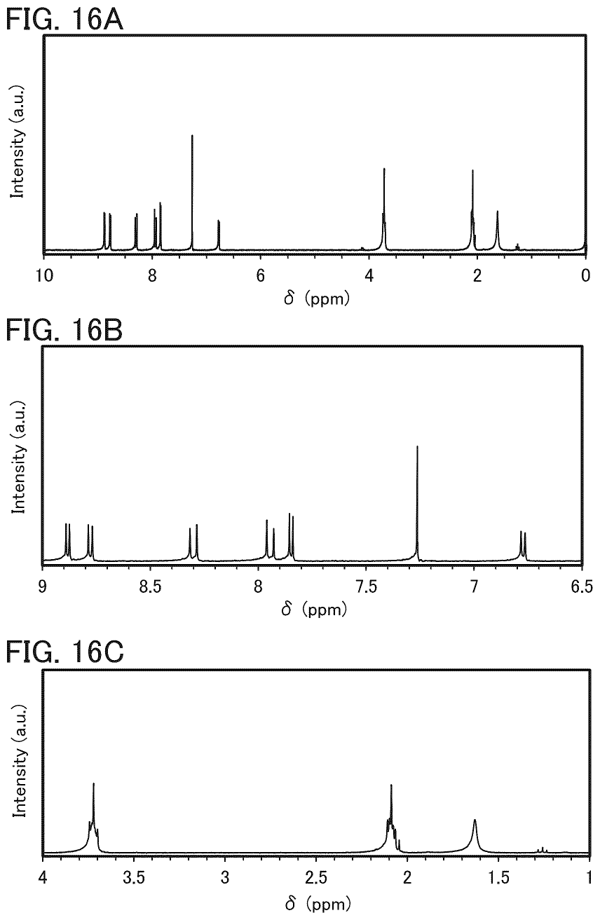

FIGS. 16A to 16C show the ¹H NMR spectrum of the yellow solid obtained. FIG. 16B is an enlarged graph of FIG. 16A in the range from 6.5 ppm to 9.0 ppm, and FIG. 16C is an enlarged graph of FIG. 16A in the range from 1 ppm to 4.0 ppm. Results of ¹H NMR measurement are shown below. The results reveal that 4Br7PrdPhen was obtained.

¹H NMR (CDCl₃, 300 MHz): δ=8.88 (1H, d, J=4.8 Hz), 8.78 (1H, d, J=5.5 Hz), 8.30 (1H, d, J=9.5 Hz), 7.94 (1H, d, J=9.5 Hz), 7.85 (1H, d, J=4.8 Hz), 6.77 (1H, d, J=5.9 Hz), 3.74-3.70 (4H, m), 2.11-2.07 (4H, m).

### Example 5

### Synthesis Example 5

Described in this synthesis example is a method for synthesizing 2-chloro-9-(2,3,3a,4,5,6,7,7a-octahydro-1H-isoindol-2-yl)-1,10-phenanthroline (abbreviation: 2Cl9HidPhen), which is the organic compound of the present invention that is represented by Structural Formula (203) in Embodiment 1. The structure of 2Cl9HidPhen is shown below.

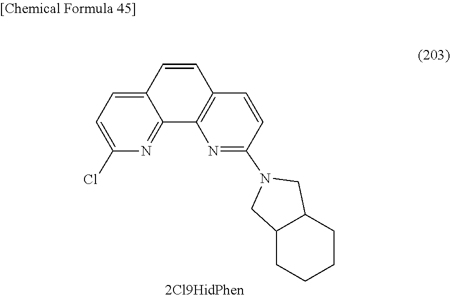 [SMILES: Clc1ccc2ccc3ccc(N4CC5CCCCC5C4)nc3c2n1]

Into a standard glass reaction vessel with a diameter of 24 mm were put 1.0 g (4.0 mmol) of 2,9-dichloro-1,10-phenanthroline, 1.1 mL (8.4 mmol) of 2,3,3a,4,5,6,7,7a-octahydro-1H-isoindole, 1.7 g (12 mmol) of potassium carbonate (abbreviation: K₂CO₃), and 8 mL of N-methylpyrrolidone (abbreviation: NMP). The mixture was stirred at 80° C. for 4 hours. After the mixture was air-cooled to room temperature and water was added to the mixture, the mixture was subjected to extraction with chloroform. The extracted solution was washed with a saturated aqueous solution of sodium chloride once. After the separation, the organic layer was gravity-filtered, and the filtrate was concentrated to give a precipitated yellow solid. Suction filtration was performed to give 1.3 g of a yellow solid containing a target substance. This solid was purified by silica gel column chromatography (developing solvent: chloroform) to give a target yellow solid (0.67 g in a yield of 50%). Synthesis Scheme (s5-1) of this synthesis example is shown below.

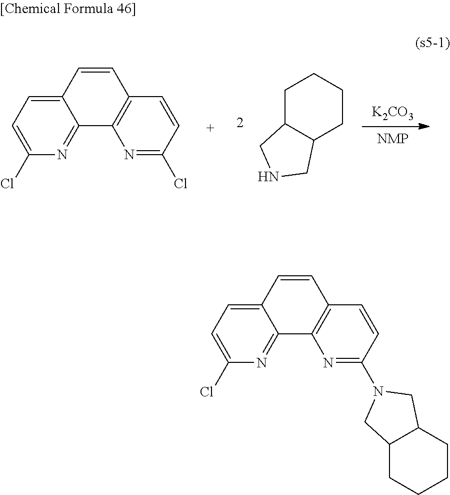

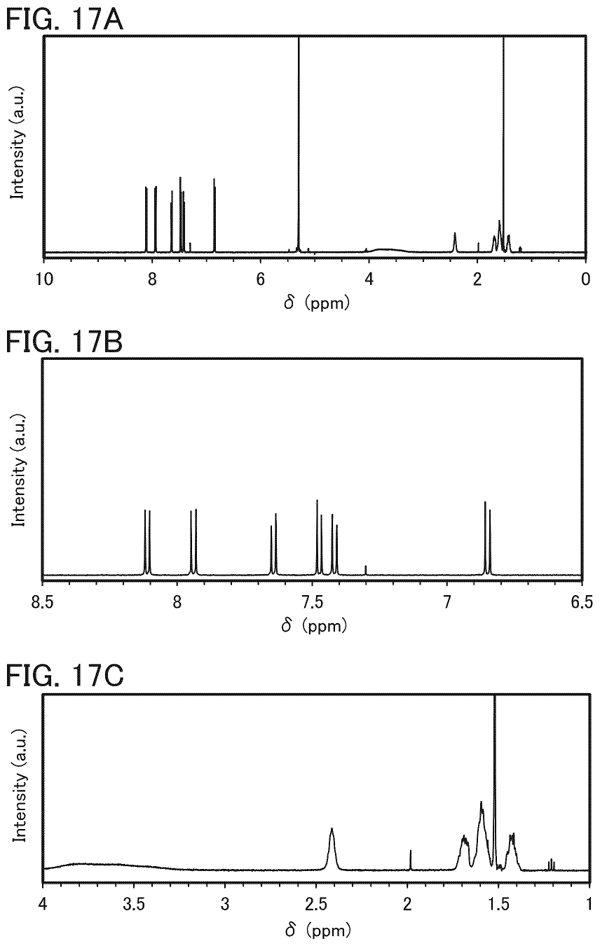

FIGS. 17A to 17C show the ¹H NMR measurement result of the yellow solid obtained. FIG. 17B is an enlarged graph of FIG. 17A in the range from 6.5 ppm to 8.5 ppm, and FIG. 17C is an enlarged graph of FIG. 17A in the range from 1 ppm to 4.0 ppm. Results of ¹H NMR measurement are shown below.

¹H NMR (CD₂Cl₂, 500 MHz): δ=8.11 (1H, d, J=8.5 Hz), 7.94 (1H, d, J=9.5 Hz), 7.64 (1H, d, J=8.5 Hz), 7.47 (1H, d, J=8.0 Hz), 7.42 (1H, d, J=8.5 Hz), 6.75 (1H, d, J=9.0 Hz), 4.20-3.00 (8H, br), 2.50-2.35 (4H, br), 1.76-1.35 (16H, m).

The molecular weight of the yellow solid obtained was measured by LC/MS analysis. As a result, a peak indicating m/z 338 was observed while the mass of the target substance was calculated to be 337. This is probably a peak indicating a proton adduct of 2Cl9HidPhen.

The ¹H NMR and LC/MS analysis results indicate that 2Cl9HidPhen was obtained in this synthesis example.

### Example 6

### Synthesis Example 6

Described in this synthesis example is a method for synthesizing 4-chloro-4′-(2,3,3a,4,5,6,7,7a-octahydro-1H-isoindol-2-yl)-2,2′-bipyridine (abbreviation: 4Cl4′HidBpy), which is the organic compound of the present invention that is represented by Structural Formula (204) in Embodiment 1. The structure of 4Cl4′HidBpy is shown below.

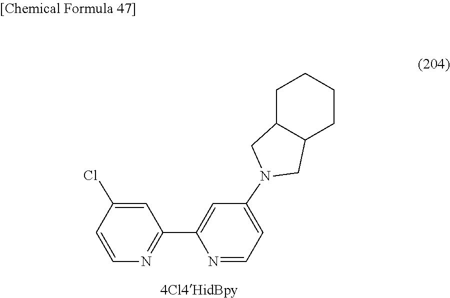 [SMILES: Clc1ccnc(-c2cc(N3CC4CCCCC4C3)ccn2)c1]

Into a standard glass reaction vessel with a diameter of 24 mm were put 1.0 g (4.4 mmol) of 4,4′-dichloro-2,2′-bipyridine, 1.1 mL (9.0 mmol) of 2,3,3a,4,5,6,7,7a-octahydro-1H-isoindole, 1.8 g (13 mmol) of potassium carbonate (abbreviation: K₂CO₃), and 9 mL of N-methylpyrrolidone (abbreviation: NMP). The mixture was stirred at 80° C. for 6 hours. After the mixture was air-cooled to room temperature and water was added to the mixture, the mixture was subjected to extraction with chloroform. The extract layer was gravity-filtered, and then the filtrate was concentrated to give an oily substance. The oily substance was purified by silica gel column chromatography (developing solvent: ethyl acetate, chloroform, and ethanol in this order). The resulting fraction was concentrated to give an oily substance. A small amount of chloroform and a small amount of hexane were added to the oily substance, irradiation with ultrasonic waves was performed, and the precipitated solid was collected by suction filtration to give a target white solid (0.45 g in a yield of 32%). Synthesis Scheme (s6-1) of this synthesis example is shown below.

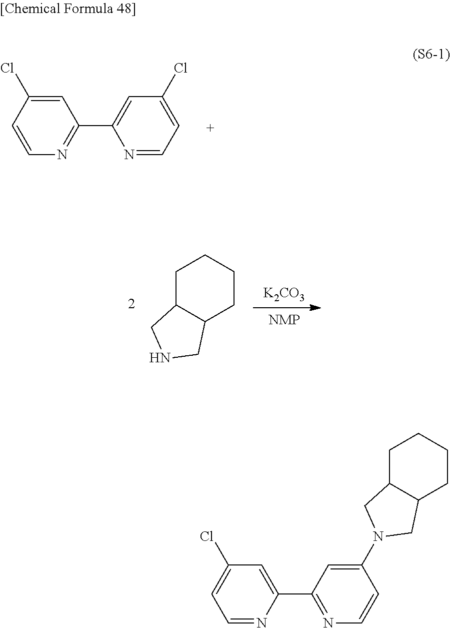

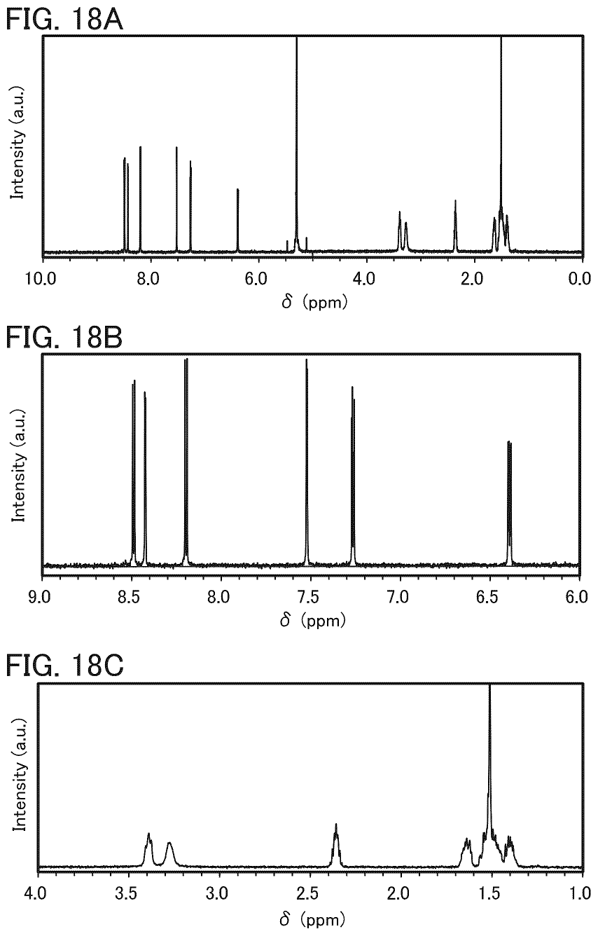

FIGS. 18A to 18C show the ¹H NMR measurement result of the white solid obtained. FIG. 18B is an enlarged graph of FIG. 18A in the range from 6.0 ppm to 9.0 ppm, and FIG. 18C is an enlarged graph of FIG. 18A in the range from 1 ppm to 4.0 ppm. Results of ¹H NMR measurement are shown below.

¹H NMR (CD₂Cl₂, 500 MHz): δ=8.49 (1H, d, J=5.0 Hz), 8.43 (1H, d, J=2.5 Hz), 8.20 (1H, d, J=5.0 Hz), 7.52 (1H, d, J=2.0 Hz), 7.27 (1H, dd, J=5.3 Hz, 2.0 Hz), 6.39 (1H, dd, J=6.0 Hz, 3.0 Hz), 3.46-3.20 (4H, m), 2.35 (2H, m), 1.70-1.34 (8H, m).

The molecular weight of the white solid obtained was measured by LC/MS analysis. As a result, a peak indicating m/z 314 was observed while the mass of the target substance was calculated to be 313. This is probably a peak of a proton adduct of 4Cl4′HidBpy.

The ¹H NMR and LC/MS analysis results indicate that 4Cl4′HidBpy was obtained.

### Example 7

### Synthesis Example 7

Described in this example is a method for synthesizing 4-(2,3,3a,4,5,6,7,7a-octahydro-1H-isoindol-2-yl)-7-(3,4-diphenyl-1-pyrrolidinyl)-1,10-phenanthroline (abbreviation: Hid-DPPrdPhen) represented by Structural Formula (100) in Embodiment 1. The structural of Hid-DPPrdPhen is shown below.

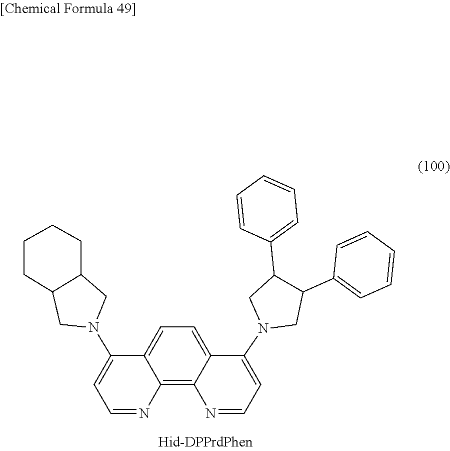 [SMILES: c1ccc(C2CN(c3ccnc4c3ccc3c(N5CC6CCCCC6C5)ccnc34)CC2c2ccccc2)cc1]

Into a 50-mL three-neck flask were put 2.8 g (8.3 mmol) of 4-chloro-7-(2,3,3a,4,5,6,7,7a-octahydro-1H-isoindol-2-yl)-1,10-phenanthroline, 2.0 g (9.0 mmol) of rac-trans-3,4-diphenylpyrrolidine, and 3.7 g (24 mmol) of diazabicycloundecene (DBU). The mixture was stirred at 100° C. under a nitrogen stream for 8 hours. After the stirring, the mixture was cooled down to room temperature. After water was added to the mixture, the mixture was subjected to extraction with chloroform. The extracted solution was concentrated to give an oily substance. Ethyl acetate and hexane were added to the oily substance, irradiation with ultrasonic waves was performed, and the solid was collected by suction filtration to give a target light brown solid (2.5 g in a yield of 58%). The synthesis scheme of this synthesis example is shown in Formula (s7-1) below.

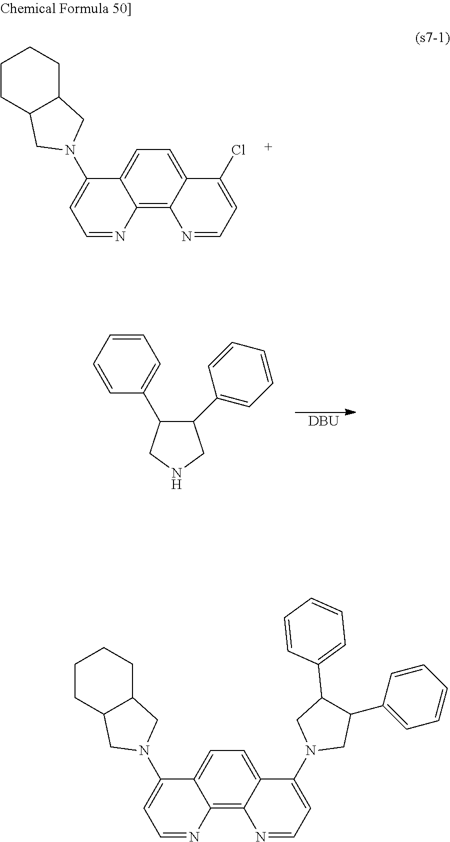

By a train sublimation method, 1.3 g out of 2.5 g of the light brown solid obtained was purified. In the purification by sublimation, heating was performed for 24 hours at an argon flow rate of 0 mL/min, a pressure of 2.8×10⁻² Pa, and a heating temperature of 255° C. As a result, a target yellow solid was obtained (0.53 g at a collection rate of 41%).

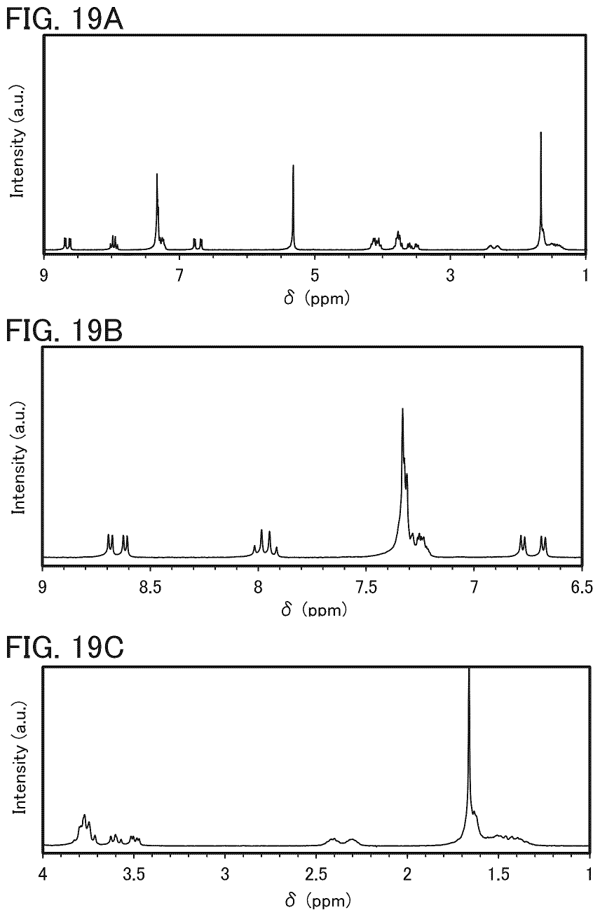

FIGS. 19A to 19C show the ¹H NMR spectrum of the yellow solid obtained. FIG. 19B is an enlarged graph of FIG. 19A in the range from 6.5 ppm to 9.0 ppm, and FIG. 19C is an enlarged graph of FIG. 19A in the range from 1 ppm to 4.0 ppm. Results of ¹H NMR measurement are shown below. The results reveal that Hid-DPPrdPhen was obtained in this synthesis example.

¹H NMR (CD₂Cl₂, 300 MHz): δ=8.68 (1H, d, J=5.1 Hz), 8.62 (1H, d, J=5.5 Hz), 8.02-7.92 (2H, m), 7.33-7.21 (10H, m), 6.77 (1H, d, J=5.1 Hz), 6.68 (1H, d, J=5.5 Hz), 4.17-4.02 (4H, m), 3.83-3.71 (4H, m), 3.63-3.57 (1H, m), 3.52-3.47 (1H, m), 2.44-2.37 (1H, m), 2.33-2.27 (1H, m), 1.63-1.32 (8H, m).

The glass transition temperature (T_{g}) of Hid-DPPrdPhen was measured. Note that T_{g} was measured with a differential scanning calorimeter (DSC8500, PerkinElmer Japan Co., Ltd.) in a state where a powder was put on an aluminum cell and the temperature was increased at a rate of 40° C./min. The result reveals that Hid-DPPrdPhen has a T_{g} of 129° C. and has high heat resistance.

Next, a solubility test of Hid-DPPrdPhen was performed. Note that the solubility test was conducted at a pressure of one atmosphere at room temperature (RT).

### <Solubility Test of Hid-DPPrdPhen by LC/MS Analysis>

In the LC/MS analysis, liquid chromatography (LC) separation was carried out with ACQUITY UPLC (Waters Corporation), and mass spectrometry (MS) analysis was carried out with Xevo G2 Tof MS (Waters Corporation). ACQUITY UPLC BEH C8 (2.1×100 mm, 1.7 μm) was used as a column for the LC separation. Acetonitrile was used for Mobile Phase A, and a 0.1% aqueous solution of formic acid was used for Mobile Phase B. The injection amount of the sample was 5.0 μL. Note that in the analysis, the wavelength of a photodiode array detector was set to 254 nm±1 nm.

In a 5-mL sample bottle, 1 mg of Hid-DPPrdPhen was put and 1 mL of water was added thereto. The mixture was irradiated with ultrasonic waves for 5 minutes. The mixture was filtered through a membrane filter to remove the solid, and the resulting filtrate was diluted by 5 times with acetonitrile. The solution was subjected to LC/MS analysis.

As a result, the peak area value derived from Hid-DPPrdPhen failed to be obtained through the LC/MS analysis.

The above result reveals that Hid-DPPrdPhen, which is the organic compound of one embodiment of the present invention, has low solubility in water and can be suitably used for a light-emitting device whose fabrication process includes processing using water or a chemical solution containing water as a solvent (i.e., a light-emitting device involving processing by a lithography method).

### Example 8

### Synthesis Example 8

Described in this synthesis example is a method for synthesizing 4-(2,3,3a,4,5,6,7,7a-octahydro-1H-isoindol-2-yl)-7-[3-(1-naphthyl)-1-pyrrolidinyl]-1,10-phenanthroline (abbreviation: Hid-αNPrdPhen) represented by Structural Formula (101) in Embodiment 1. The structure of Hid-αNPrdPhen is shown below.

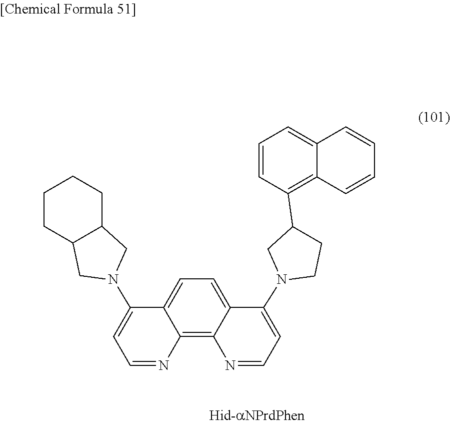 [SMILES: c1ccc2c(C3CCN(c4ccnc5c4ccc4c(N6CC7CCCCC7C6)ccnc45)C3)cccc2c1]

Into a 50-mL three-neck flask were put 2.6 g (7.7 mmol) of 4-chloro-7-(2,3,3a,4,5,6,7,7a-octahydro-1H-isoindol-2-yl)-1,10-phenanthroline, 2.0 g (8.6 mmol) of 3-(naphthalen-1-yl)pyrrolidine hydrochloride, and 3.6 g (24 mmol) of diazabicycloundecene (DBU). The mixture was stirred at 100° C. under a nitrogen stream for 8 hours. After the stirring, the mixture was cooled down to room temperature. After water was added to the mixture, the solution was subjected to extraction with chloroform. The extracted solution was concentrated to give an oily substance. Acetone was added to the oily substance, irradiation with ultrasonic waves was performed, and the solid was collected by suction filtration to give a target light red solid (3.6 g in a yield of 92%). The synthesis scheme of this synthesis example is shown in Formula (s8-1) below.

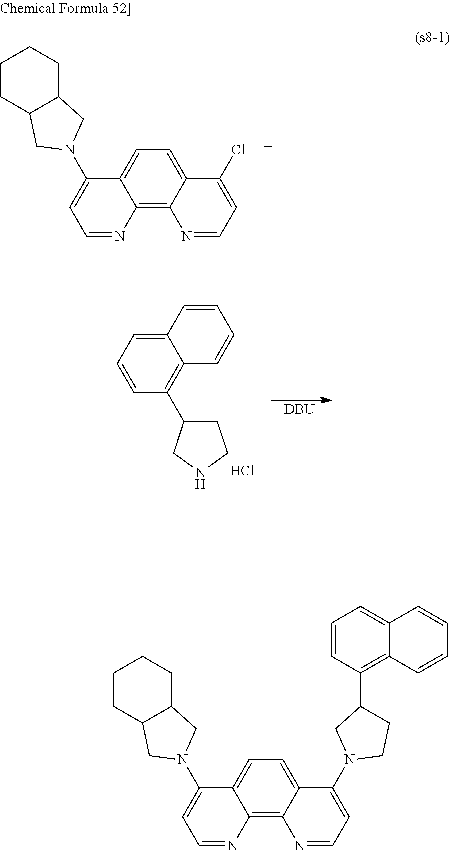 [SMILES: Clc1ccnc2c1ccc1c(N3CC4CCCCC4C3)ccnc12 ; c1ccc2c(C3CCNC3)cccc2c1 ; Cl ; "DBU" ; c1ccc2c(C3CCN(c4ccnc5c4ccc4c(N6CC7CCCCC7C6)ccnc45)C3)cccc2c1]

By a train sublimation method, 2.1 g out of 3.6 g of the light red solid obtained was purified by sublimation. In the purification by sublimation, heating was performed for 24 hours at an argon flow rate of 0 mL/min, a pressure of 4.5×10⁻² Pa, and a heating temperature of 255° C. As a result, a target yellow solid was obtained (0.20 g at a collection rate of 9.5%).

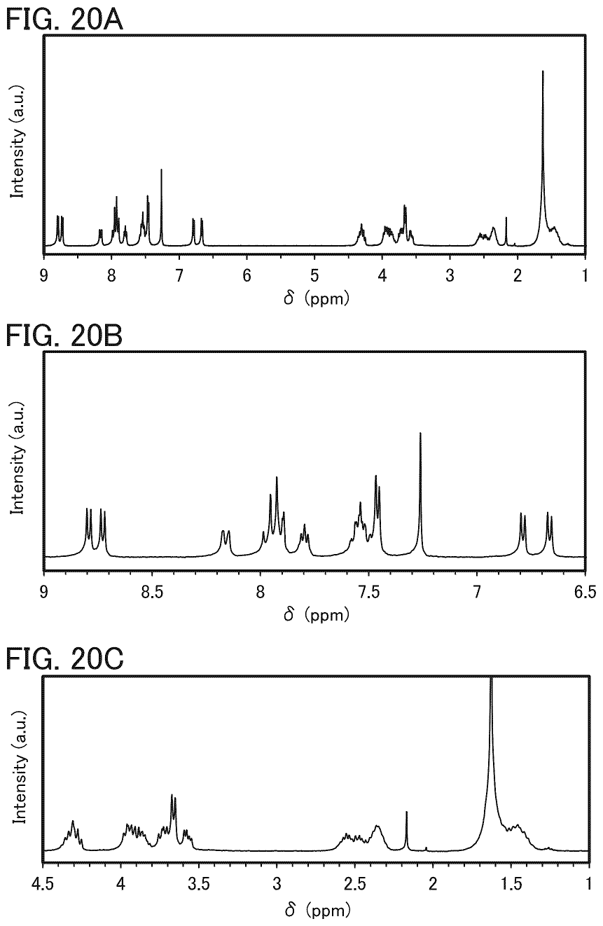

FIGS. 20A to 20C show the ¹H NMR spectrum of the yellow solid obtained. FIG. 20B is an enlarged graph of FIG. 20A in the range from 6.5 ppm to 9.0 ppm, and FIG. 20C is an enlarged graph of FIG. 20A in the range from 1.0 ppm to 4.5 ppm. Results of ¹H NMR measurement are shown below. The results reveal that Hid-αNPrdPhen was obtained in this synthesis example.

¹H NMR (CDCl₃, 300 MHz): δ=8.79 (1H, d, J=5.5 Hz), 8.73 (1H, d, J=5.5 Hz), 8.16 (1H, d, J=7.7 Hz), 7.99-7.89 (3H, m), 7.80 (1H, t, J=4.6 Hz), 7.58-7.45 (4H, m), 6.79 (1H, d, J=5.5 Hz), 6.67 (1H, d, J=5.5 Hz), 4.38-4.25 (2H, m), 3.98-3.82 (3H, m), 3.76-3.55 (4H, m), 2.59-2.32 (4H, m), 1.63-1.39 (8H, m).

The glass transition temperature (T_{g}) of Hid-αNPrdPhen was measured. Note that T_{g} was measured with a differential scanning calorimeter (DSC8500, PerkinElmer Japan Co., Ltd.) in a state where a powder was put on an aluminum cell and the temperature was increased at a rate of 40° C./min. The result reveals that Hid-αNPrdPhen has a T_{g} of 125° C.

Next, a solubility test of Hid-αNPrdPhen was performed. Note that the solubility test was conducted at a pressure of one atmosphere at room temperature (RT).

### <Solubility Test of Hid-αNPrdPhen by LC/MS Analysis>

In the LC/MS analysis, liquid chromatography (LC) separation was carried out with ACQUITY UPLC (Waters Corporation), and mass spectrometry (MS) analysis was carried out with Xevo G2 Tof MS (Waters Corporation). ACQUITY UPLC BEH C8 (2.1×100 mm, 1.7 μm) was used as a column for the LC separation. Acetonitrile was used for Mobile Phase A, and a 0.1% aqueous solution of formic acid was used for Mobile Phase B. The injection amount of the sample was 5.0 μL. Note that in the analysis, the wavelength of a photodiode array detector was set to 254 nm±1 nm.

In a 5-mL sample bottle, 1 mg of Hid-αNPrdPhen was put and 2 mL of chloroform was added thereto. The mixture was irradiated with ultrasonic waves for 10 minutes. After it was confirmed that the solid was completely dissolved, the solution was diluted by 2.5 times with acetonitrile to prepare a solution with a concentration of 0.20 g/L. This solution was diluted with acetonitrile to prepare a solution with a concentration of 2.5 mg/L and a solution with a concentration of 0.5 mg/L. The prepared solutions were subjected to LC/MS analysis, and the peak area values derived from Hid-αNPrdPhen, which were obtained at the respective solution concentrations, were used to form calibration curves.

Next, the solubility of Hid-αNPrdPhen in water was measured.

In a 5-mL sample bottle, 1 mg of Hid-αNPrdPhen was put and 1 mL of water was added thereto. The mixture was irradiated with ultrasonic waves for 5 minutes. The mixture was filtered through a membrane filter to remove the solid, and the resulting filtrate was diluted by 5 times with acetonitrile. The solution was subjected to LC/MS analysis.

From the calibration curves and the signal intensity obtained by the LC/MS analysis, it was found that the solubility of Hid-αNPrdPhen in 1 mL of water is 0.0023 mg. Note that the solubility of Hid-αNPrdPhen in water can be converted to a weight fraction of 2.3×10⁻⁶, indicating low solubility of Hid-αNPrdPhen in water.

The above result reveals that Hid-αNPrdPhen, which is the organic compound of one embodiment of the present invention, has low solubility in water and can be suitably used for a light-emitting device whose fabrication process includes processing using water or a chemical solution containing water as a solvent (i.e., a light-emitting device involving processing by a lithography method).

### Example 9

### Synthesis Example 9

Described in this synthesis example is a method for synthesizing 4-(2,3,3a,4,5,6,7,7a-octahydro-1H-isoindol-2-yl)-7-[4-(1-naphthyl)phenyl]-1,10-phenanthroline (abbreviation: Hid-αNPPhen) represented by Structural Formula (103) in Embodiment 1. The structural of Hid-αNPPhen is shown below.

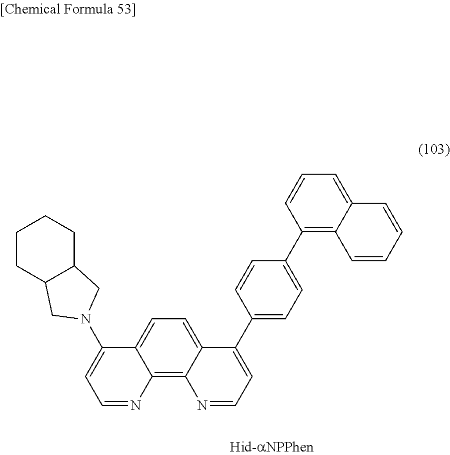 [SMILES: c1ccc2c(-c3ccc(-c4ccnc5c4ccc4c(N6CC7CCCCC7C6)ccnc45)cc3)cccc2c1]

Into a 100-mL three-neck flask were put 2.4 g (6.3 mmol) of 4-bromo-7-(2,3,3a,4,5,6,7,7a-octahydro-1H-isoindol-2-yl)-1,10-phenanthroline, 1.7 g (6.9 mmol) of 4-(1-naphthyl)phenylboronic acid, 0.16 g (0.45 mmol) of di(1-adamantyl)-n-butylphosphine, 3.4 g (10 mmol) of cesium carbonate, 36 mL of 1,4-dioxane, and 13 mL of water. The mixture was degassed by being stirred under reduced pressure. To this mixture was added 0.17 g (0.19 mmol) of tris(dibenzylideneacetone)dipalladium(0). The mixture was stirred at 100° C. under a nitrogen stream for 30 hours. After the stirring, the mixture was cooled down to room temperature. After water was added to the mixture, the mixture was subjected to extraction with dichloromethane. The extracted solution was concentrated to give an oily substance. The oily substance was purified by silica gel column chromatography (developing solvent: chloroform and methanol in this order). The resulting fraction was concentrated to give an oily substance. A small amount of chloroform and a small amount of ethyl acetate were added to the oily substance, irradiation with ultrasonic waves was performed, and the precipitated solid was collected by suction filtration to give a target light brown solid (1.2 g in a yield of 39%). The synthesis scheme of this synthesis example is shown in Formula (s9-1) below.

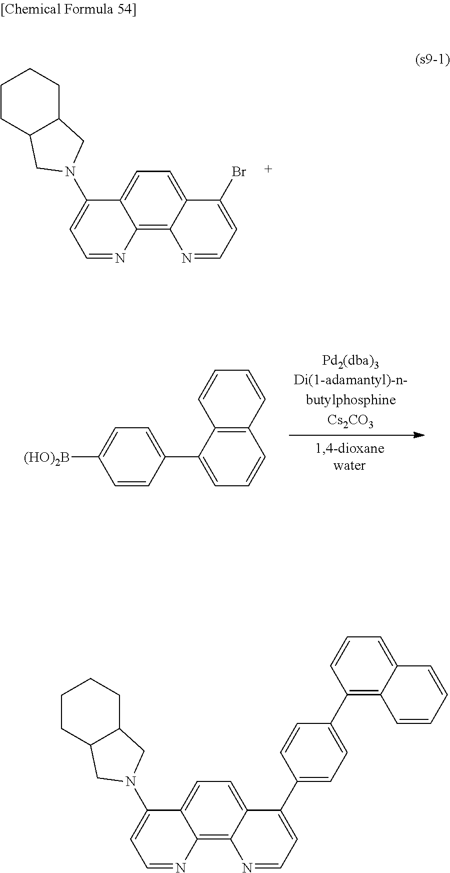 [SMILES: Brc1ccnc2c1ccc1c(N3CC4CCCCC4C3)ccnc12 ; OB(O)c1ccc(-c2cccc3ccccc23)cc1 ; c1ccc2c(-c3ccc(-c4ccnc5c4ccc4c(N6CC7CCCCC7C6)ccnc45)cc3)cccc2c1]

By a train sublimation method, 1.2 g of the light brown solid obtained was purified by sublimation. In the purification by sublimation, heating was performed for 43 hours at an argon flow rate of 0 mL/min, a pressure of 3.2×10⁻² Pa, and a heating temperature of 240° C. As a result, a target yellow solid was obtained (0.55 g at a collection rate of 46%).

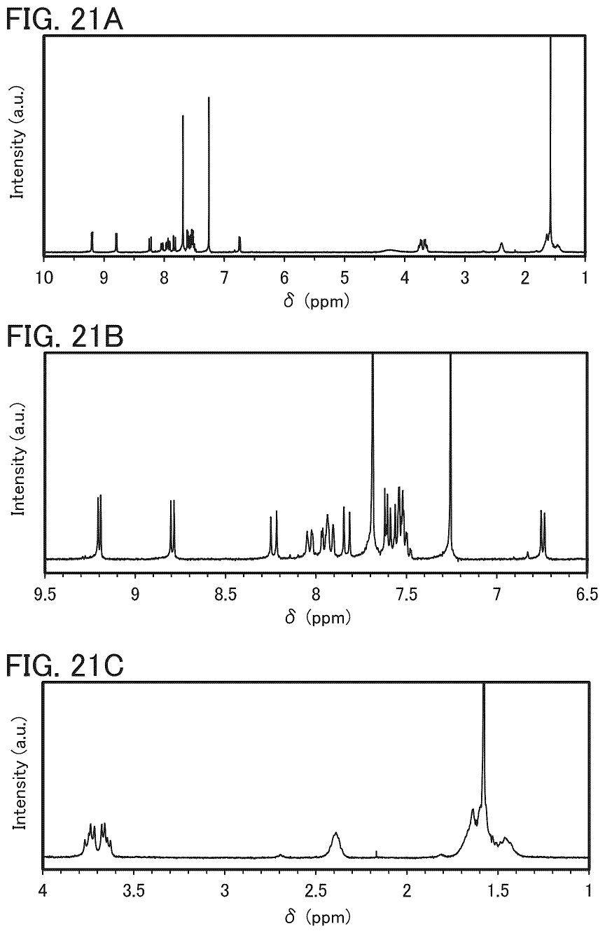

FIGS. 21A to 21C show the ¹H NMR spectrum of the yellow solid obtained. FIG. 21B is an enlarged graph of FIG. 21A in the range from 6.5 ppm to 9.5 ppm, and FIG. 21C is an enlarged graph of FIG. 21A in the range from 1.0 ppm to 4.0 ppm. Results of ¹H NMR measurement are shown below. The results reveal that Hid-αNPPhen was obtained.

¹H NMR (CDCl₃, 300 MHz): δ=9.20 (1H, d, J=4.4 Hz), 8.79 (1H, d, J=5.5 Hz), 8.23 (1H, d, J=9.9 Hz), 8.04 (1H, d, J=7.3 Hz), 7.97-7.91 (2H, m), 7.83 (1H, d, J=9.5 Hz), 7.69 (4H, s), 7.62-7.48 (5H, m), 6.75 (1H, d, J=5.9 Hz), 3.77-3.63 (4H, m), 2.39 (2H, br), 1.64-1.46 (8H, m).

The glass transition temperature (T_{g}) of Hid-αNPPhen was measured. Note that T_{g} was measured with a differential scanning calorimeter (DSC8500, PerkinElmer Japan Co., Ltd.) in a state where a powder was put on an aluminum cell and the temperature was increased at a rate of 40° C./min. The result reveals that Hid-αNPPhen has a T_{g} of 124° C. and has high heat resistance.

### Example 10

### Synthesis Example 10

Described in this synthesis example is a method for synthesizing 4-(6,7-dihydro-5H-dibenzo[c,e]azepin-5-yl)-7-(2,3,3a,4,5,6,7,7a-octahydro-1H-isoindol-2-yl)-1,10-phenanthroline (abbreviation: Hid-ceHBazPhen) represented by Structural Formula (102) in Embodiment 1. The structure of Hid-ceHBazPhen is shown below.

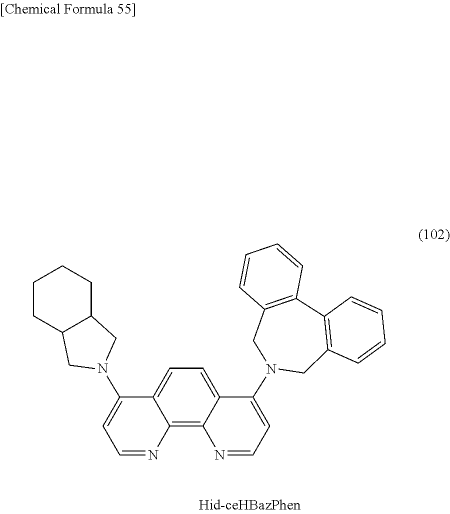 [SMILES: c1ccc2c(c1)CN(c1ccnc3c1ccc1c(N4CC5CCCCC5C4)ccnc13)Cc1ccccc1-2]

Into a 100-mL three-neck flask were put 3.4 g (10 mmol) of 4-chloro-7-(2,3,3a,4,5,6,7,7a-octahydro-1H-isoindol-2-yl)-1,10-phenanthroline, 2.6 g (11 mmol) of 6,7-dihydro-5H-dibenzo[c,e]azepine hydrochloride, and 4.6 g (30 mmol) of diazabicycloundecene (DBU). The mixture was stirred at 100° C. under a nitrogen stream for 32 hours. After the stirring, the mixture was cooled down to room temperature. After water was added to the mixture, the mixture was subjected to extraction with chloroform. The extracted solution was concentrated to give an oily substance. Ethyl acetate was added to the oily substance, irradiation with ultrasonic waves was performed, the precipitated solid was separated by suction filtration, and the filtrate was concentrated to give an oily substance. A small amount of ethyl acetate was added to the oily substance, irradiation with ultrasonic waves was performed, and the precipitated solid was collected by suction filtration to give a target light brown solid (2.1 g in a yield of 42%). The synthesis scheme of this synthesis example is shown in Formula (s10-1) below.

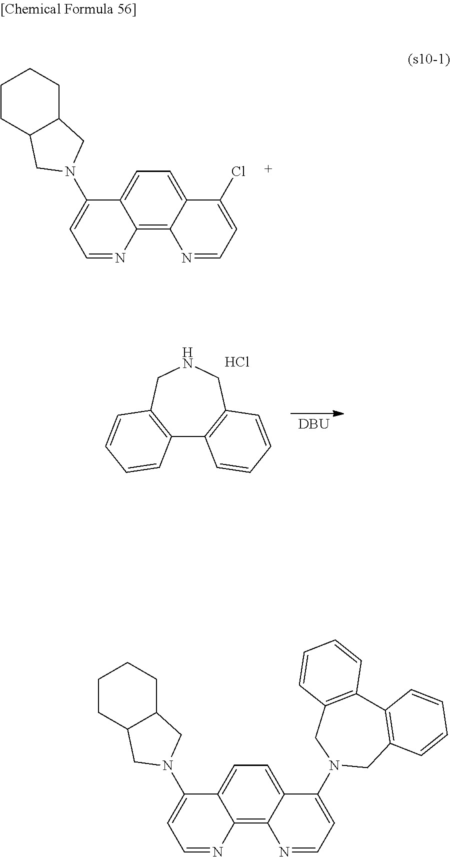

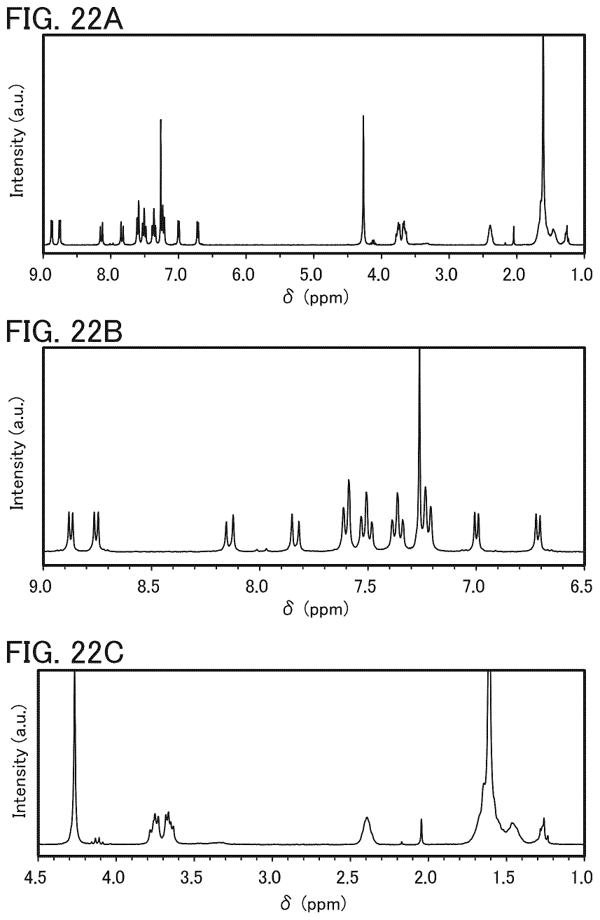

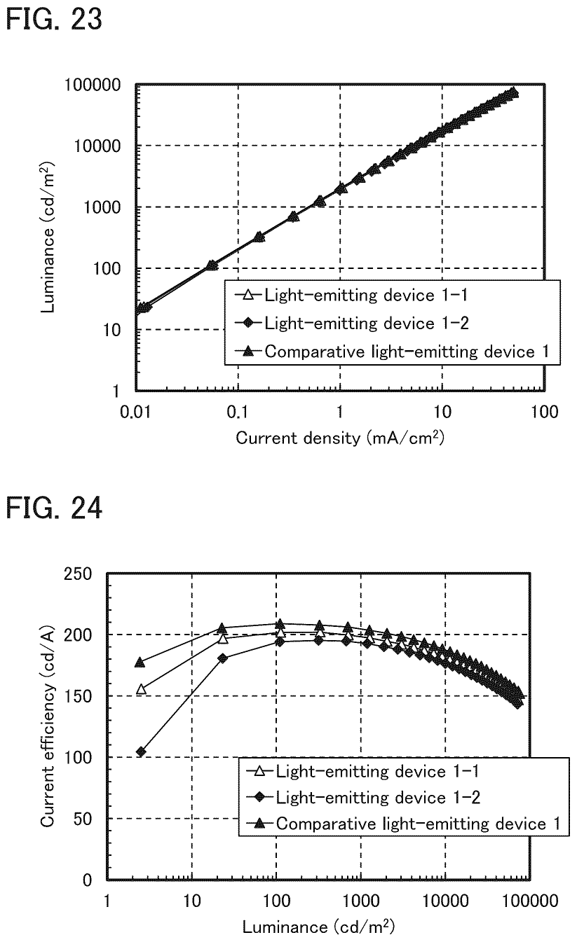

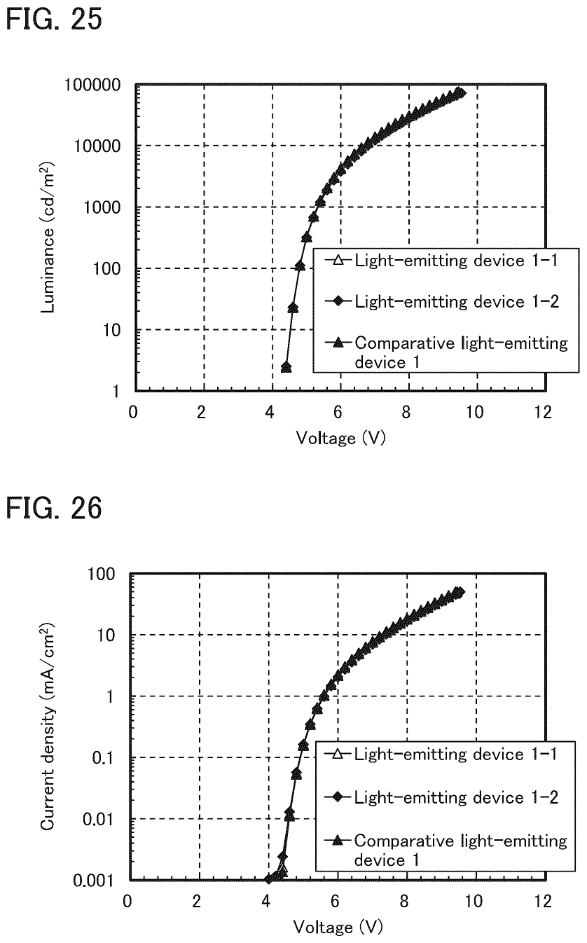

FIGS. 22A to 22C show the ¹H NMR spectrum of the light brown solid obtained. FIG. 22B is an enlarged graph of FIG. 22A in the range from 6.5 ppm to 9.0 ppm, and FIG. 22C is an enlarged graph of FIG. 22A in the range from 1.0 ppm to 4.5 ppm. Results of ¹H NMR measurement are shown below. The results reveal that Hid-ceHBazPhen was obtained.

¹H NMR (CDCl₃, 300 MHz): δ=8.87 (1H, d, J=5.1 Hz), 8.75 (1H, d, J=5.5 Hz), 8.14 (1H, d, J=9.5 Hz), 7.83 (1H, d, J=9.5 Hz), 7.60 (2H, d, J=7.3 Hz), 7.51 (2H, t, J=7.5 Hz), 7.36 (2H, t, J=7.3 Hz), 7.22 (2H, d, J=7.3 Hz), 7.00 (1H, d, J=5.1 Hz), 6.71 (1H, d, J=5.5 Hz), 4.27 (4H, s), 3.78-3.63 (4H, m), 2.39 (2H, br), 1.61-1.44 (8H, m).

### Example 11

In this example, a light-emitting device of one embodiment of the present invention will be described in detail. Structural Formulae of main organic compounds used in this example are shown below.

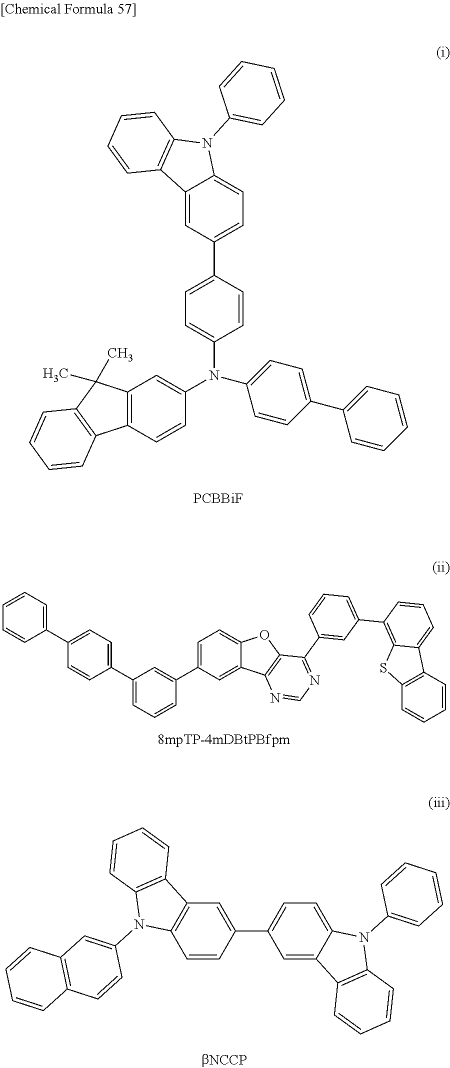

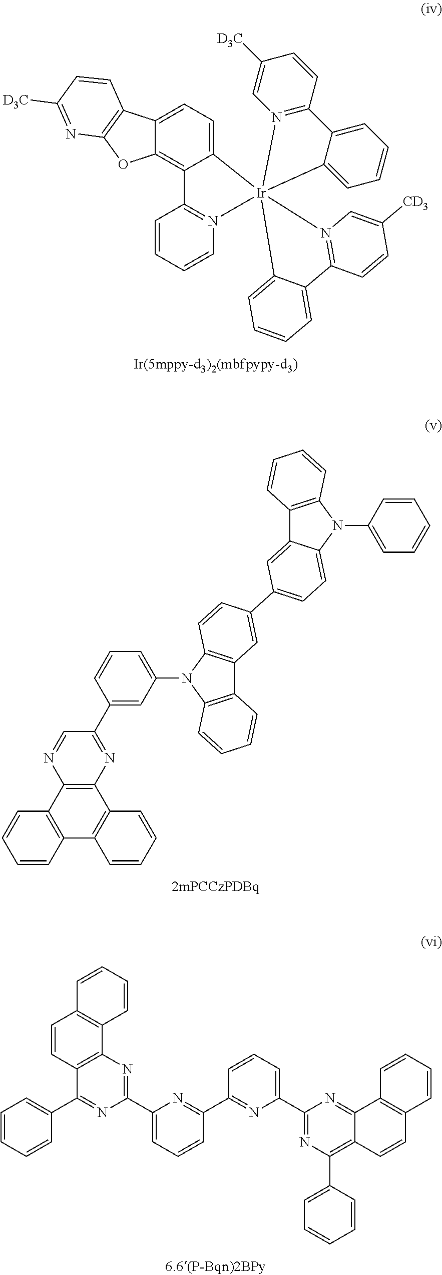

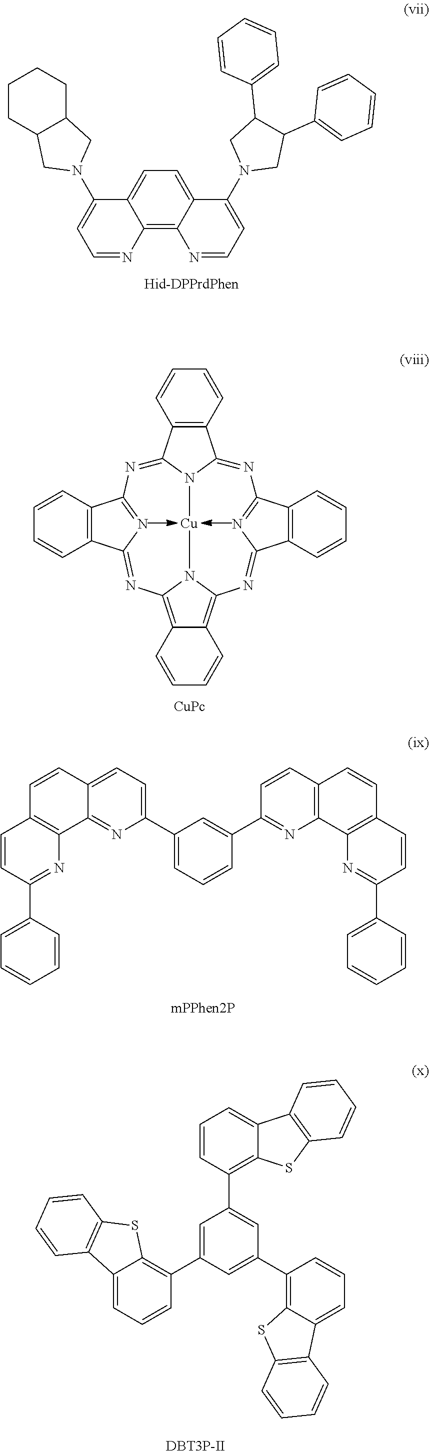

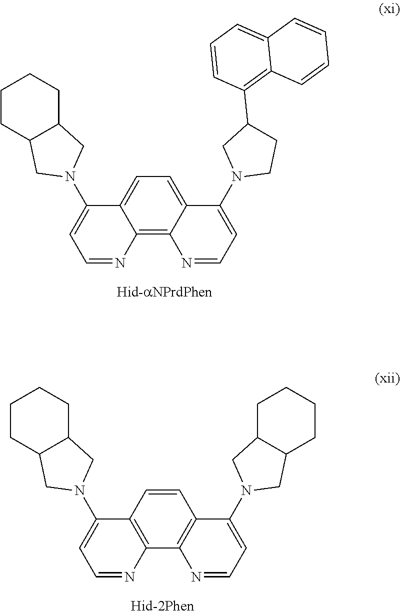 [SMILES: c1ccc2c(C3CCN(c4ccnc5c4ccc4c(N6CC7CCCCC7C6)ccnc45)C3)cccc2c1 ; c1cc(N2CC3CCCCC3C2)c2ccc3c(N4CC5CCCCC5C4)ccnc3c2n1]

### (Method for Fabricating Light-Emitting Device 1-1)

First, a 100-nm-thick film of an alloy of silver, palladium, and copper (APC: Ag—Pd—Cu) serving as a reflective electrode was formed over a substrate by a sputtering method, and then a 50-nm-thick film of indium tin oxide containing silicon oxide (JTSO) serving as a transparent electrode was stacked by a sputtering method, whereby the first electrode 101 with a size of 2 mm×2 mm was formed. Note that the transparent electrode functions as an anode, and the transparent electrode and the reflective electrode are collectively regarded as the first electrode 101.

Next, in pretreatment for forming the light-emitting device over the substrate, the substrate surface was washed with water and baking was performed at 200° C. for 1 hour.

After that, the substrate was transferred into a vacuum evaporation apparatus where the internal pressure was reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Then, the substrate was fixed to a holder provided in the vacuum evaporation apparatus such that the surface on which the first electrode 101 was formed faced downward. Over the first electrode 101, N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) above and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer 111 was formed.

Over the hole-injection layer 111, PCBBiF was deposited by evaporation to a thickness of 110 nm, whereby a first hole-transport layer was formed.

Then, over the first hole-transport layer, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (ii) above, 9-(2-naphthyl)-9′-phenyl-3,3′-bi-9H-carbazole (abbreviation: βNCCP) represented by Structural Formula (iii) above, and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) represented by Structural Formula (iv) above were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a first light-emitting layer was formed.

Next, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (v) above was deposited by evaporation to a thickness of 10 nm, whereby a first electron-transport layer was formed.

After the first electron-transport layer was formed, 2,2′-([2,2′-bipyridine]-6,6′-diyl)bis(4-phenylbenzo[h]quinazoline) (abbreviation: 6,6′(P-Bqn)2BPy) represented by Structural Formula (vi) above, 4-(3,4-diphenyl-1-pyrrolidinyl)-7-(2,3,3a,4,5,6,7,7a-octahydro-1H-isoindol-2-yl)-1,10-phenanthroline (abbreviation: Hid-DPPrdPhen) represented by Structural Formula (vii) above, and lithium oxide (Li₂O) were deposited by co-evaporation to a thickness of 5 nm such that the volume ratio of 6,6′(P-Bqn)2BPy to Hid-DPPrdPhen to Li₂O was 0.5:0.5:0.02, whereby a first layer was formed. Then, copper phthalocyanine (abbreviation: CuPc) represented by Structural Formula (viii) above was deposited by evaporation to a thickness of 2 nm, whereby a third layer was formed. Furthermore, PCBBiF and OCHD-003 were deposited by co-evaporation to a thickness of 10 nm such that the weight ratio of PCBBiF to OCHD-003 was 1:0.15, whereby a second layer was formed. Thus, an intermediate layer was formed.

Over the intermediate layer, PCBBiF was deposited by evaporation to a thickness of 50 nm, whereby a second hole-transport layer was formed.

Over the second hole-transport layer, 8mpTP-4mDBtPBfpm, βNCCP, and Ir(5mppy-d₃)₂(mbfpypy-d₃) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a second light-emitting layer was formed.

Then, 2mPCCzPDBq was deposited by evaporation to a thickness of 20 nm, and 2,2′-(1,3-phenylene)bis(9-phenyl-1,10-phenanthroline) (abbreviation: mPPhen2P) represented by Structural Formula (ix) above was further deposited by evaporation to a thickness of 20 nm, whereby a second electron-transport layer was formed.

After that, lithium fluoride (LiF) and ytterbium (Yb) were deposited by co-evaporation to a thickness of 1.5 nm such that the volume ratio of LiF to Yb was 1:0.5, whereby an electron-injection layer was formed. Then, silver (Ag) and magnesium (Mg) were deposited by co-evaporation to a thickness of 15 nm such that the volume ratio of Ag to Mg was 1:0.1, whereby the second electrode 102 was formed. Over the second electrode 102, a film of 4,4′,4″-(benzene-1,3,5-triyl)tri(dibenzothiophene) (abbreviation: DBT3P-II) represented by Structural Formula (x) above was formed to a thickness of 70 nm as a cap layer to improve light extraction efficiency.

Then, the light-emitting device was sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. In this manner, the light-emitting device 1-1 was fabricated.

### (Method for Fabricating Light-Emitting Device 1-2)

A light-emitting device 1-2 was fabricated in a manner similar to that of the light-emitting device 1-1 except that Hid-DPPrdPhen used in the first layer of the light-emitting device 1-1 was replaced with 4-(2,3,3a,4,5,6,7,7a-octahydro-1H-isoindol-2-yl)-7-[3-(1-naphthyl)-1-pyrrolidinyl]-1,10-phenanthroline (abbreviation: Hid-αNPrdPhen) represented by Structural Formula (xi) above.

### (Method for Fabricating Comparative Light-Emitting Device 1)

A comparative light-emitting device 1 was fabricated in a manner similar to that of the light-emitting device 1-1 except that Hid-DPPrdPhen used in the first layer of the light-emitting device 1-1 was replaced with 4,7-di(2,3,3a,4,5,6,7,7a-octahydro-1H-isoindol-2-yl)-1,10-phenanthroline (abbreviation: Hid2Phen) represented by Structural Formula (xii) above.

Device structures of the light-emitting devices 1-1 and 1-2 and the comparative light-emitting device 1 are shown below.

TABLE 1

| | | | | | Comparative |
|---|---|---|---|---|---|
| | | Thickness | Light-emitting device | | light-emitting |
| | | (nm) | 1-1 | 1-2 | device 1 |
| Cap layer | | 70 | DBT3P-II | | |
| Second electrode | | 15 | Ag:Mg (1:0.1) | | |
| | | 1.5 | LiF:Yb (1:0.5) | | |
| Second electron- | 2 | 20 | mPPhen2P | | |
| transport layer | 1 | 20 | 2mPCCzPDBq | | |
| Second light-emitting layer | | 40 | 8mpTP-4mDBtPBfpm:βNCCP:Ir(5mppy-d₃)₂(mbfpypy-d₃) | | |
| | | | (0.5:0.5:0.1) | | |
| Second hole-transport layer | | 50 | PCBBiF | | |
| Intermediate | Second | 10 | PCBBiF:OCHD-003 | | |
| layer | layer | | (1:0.15) | | |
| | Third layer | 2 | CuPc | | |
| | First layer | 5 | 6,6′(P-Bqn)2BPy:*1:Li₂O | | |
| | | | (0.5:0.5:0.02) | | |
| First electron-transport layer | | 10 | 2mPCCzPDBq | | |
| First light-emitting layer | | 40 | 8mpTP-4mDBtPBfpm:βNCCP:Ir(5mppy-d₃)₂(mbfpypy-d₃) | | |
| | | | (0.5:0.5:0.1) | | |
| First hole-transport layer | | 110 | PCBBiF | | |
| Hole-injection layer | | 10 | PCBBiF:OCHD-003 | | |
| | | | (1:0.03) | | |
| First electrode | | 50 | ITSO | | |
| | | 100 | APC | | |
| Z | Z | Z | Z | Z | Z |

TABLE 2

| | *1 |
|---|---|
| Light-emitting device 1-1 | Hid-DPPrdPhen |
| Light-emitting device 1-2 | Hid-αNPrdPhen |
| Comparative light-emitting | Hid2Phen |
| device 1 | |

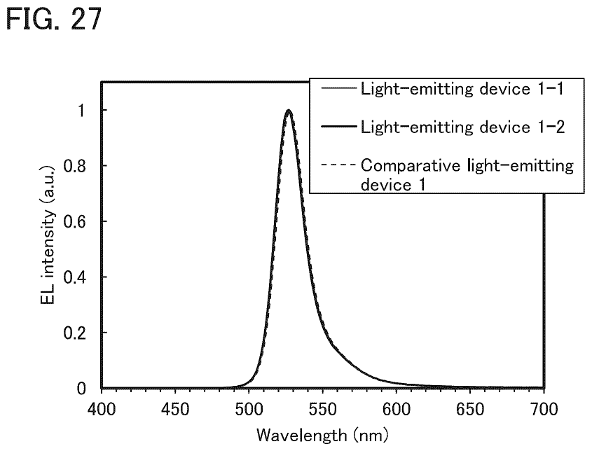

FIG. 23 shows luminance-current density characteristics of the light-emitting devices 1-1 and 1-2 and the comparative light-emitting device 1. FIG. 24 shows current efficiency-luminance characteristics thereof. FIG. 25 shows luminance-voltage characteristics thereof. FIG. 26 shows current density-voltage characteristics thereof. FIG. 27 shows electroluminescence spectra thereof. Table 3 shows the main characteristics of the light-emitting devices 1-1 and 1-2 and the comparative light-emitting device 1 at a luminance of approximately 1000 cd/m². The luminance, CIE chromaticity, and electroluminescence spectra were measured at normal temperature with a spectroradiometer SR-UL1R (TOPCON TECHNOHOUSE).

TABLE 3

| | | | Current | | | Current |
|---|---|---|---|---|---|---|
| | Voltage | Current | density | Chromaticity | Chromaticity | efficiency |
| | (V) | (mA) | (mA/cm²) | x | y | (cd/A) |
| Light-emitting device 1-1 | 5.40 | 0.0259 | 0.648 | 0.217 | 0.738 | 197 |
| Light-emitting device 1-2 | 5.40 | 0.0247 | 0.619 | 0.215 | 0.739 | 193 |
| Comparative light-emitting device 1 | 5.40 | 0.0249 | 0.623 | 0.221 | 0.737 | 204 |

FIGS. 23 to 27 and Table 2 reveal that each light-emitting device has high current efficiency and functions as a tandem light-emitting device.

The above results reveal that a light-emitting device in which the organic compound represented by General Formula (G1) in Embodiment 1 is used for a first layer in a tandem light-emitting device exhibits favorable characteristics.

### Example 12

In this example, a light-emitting device of one embodiment of the present invention will be described in detail. Structural Formulae of main organic compounds used in this example are shown below.

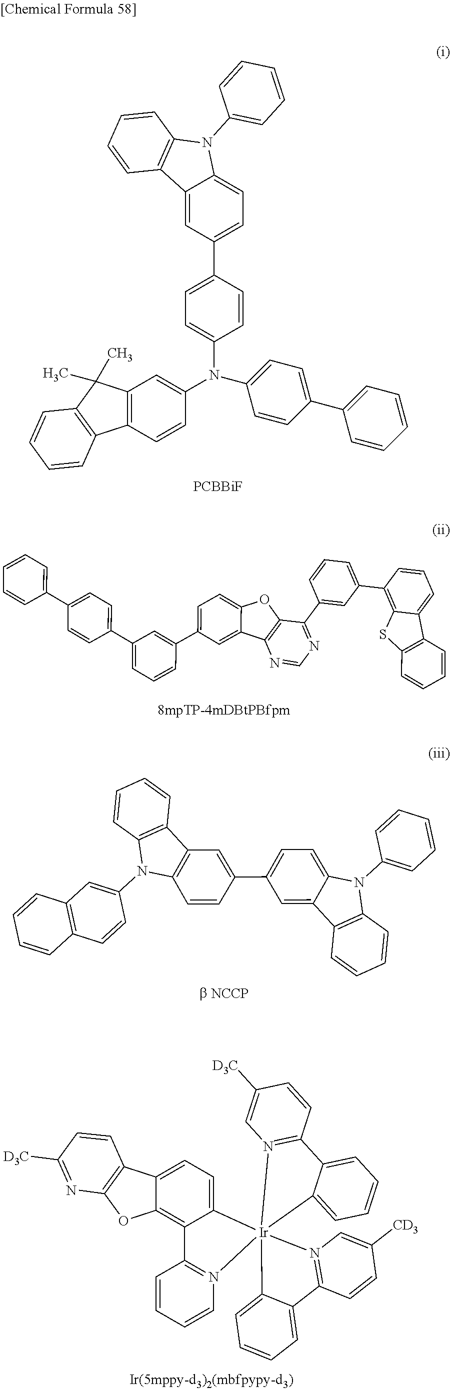

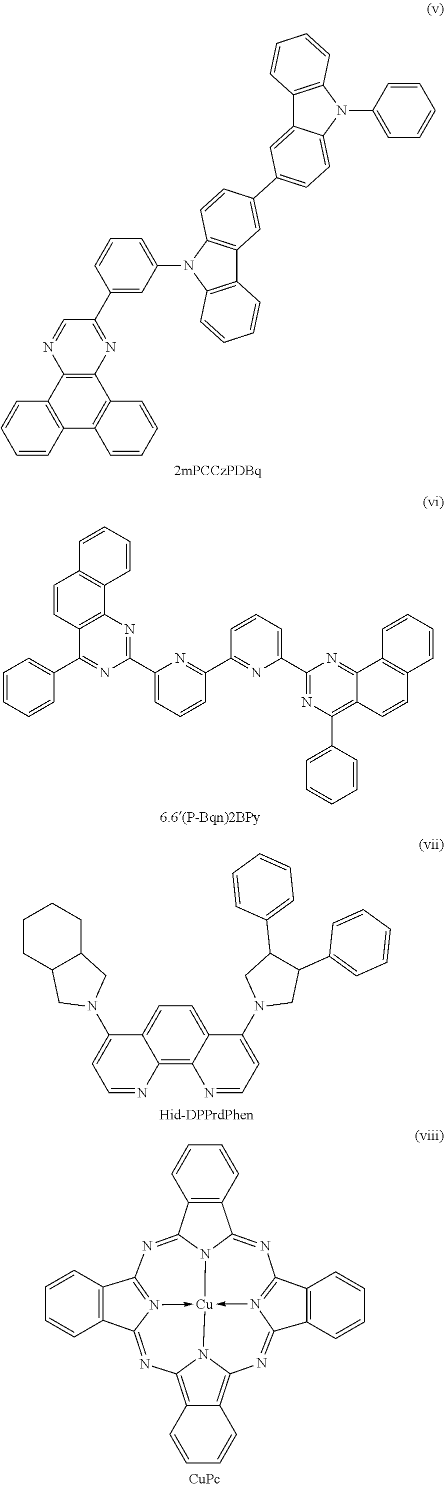

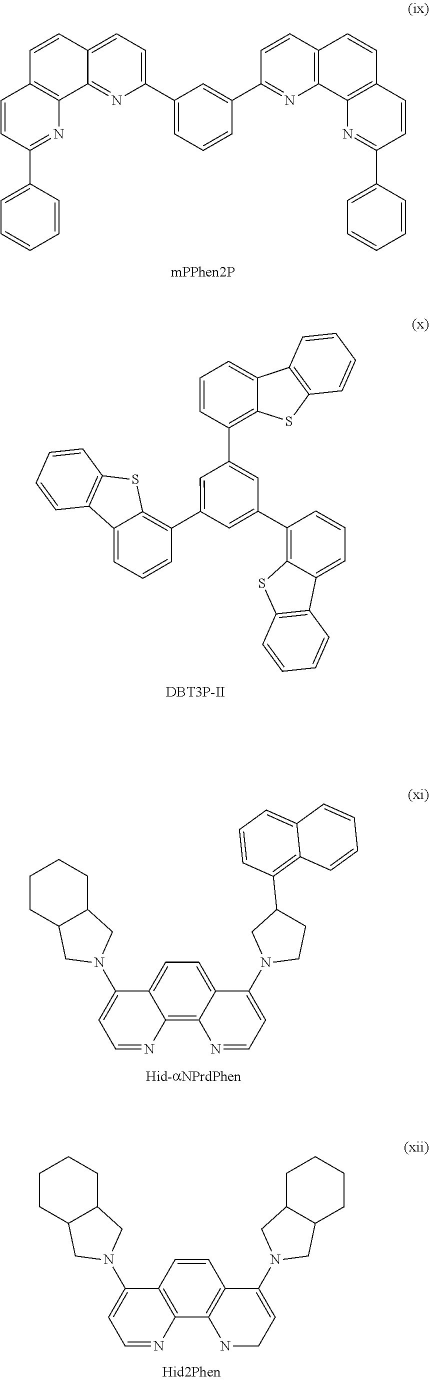 [SMILES: c1ccc(-c2ccc3ccc4ccc(-c5cccc(-c6ccc7ccc8ccc(-c9ccccc9)nc8c7n6)c5)nc4c3n2)cc1 ; c1ccc2c(c1)sc1c(-c3cc(-c4cccc5c4sc4ccccc45)cc(-c4cccc5c4sc4ccccc45)c3)cccc12 ; "DBT3P-II" ; c1ccc2c(C3CCN(c4ccnc5c4ccc4c(N6CC7CCCCC7C6)ccnc45)C3)cccc2c1 ; C1=C(N2CC3CCCCC3C2)c2ccc3c(N4CC5CCCCC5C4)ccnc3c2NC1]

### (Method for Fabricating Light-Emitting Device 2-1)

First, a 100-nm-thick film of an alloy of silver, palladium, and copper (APC: Ag—Pd—Cu) was formed over a substrate by a sputtering method, a 50-nm-thick film of indium tin oxide including silicon oxide (JTSO) serving as a transparent electrode was stacked by a sputtering method, and the stacked films were patterned by a photolithography method, whereby the first electrode was formed. As the patterned first electrode, a plurality of electrodes are provided in a region of 2 mm×2 mm at a resolution of 508 ppi and constitute a first electrode group.

Next, in pretreatment for forming the light-emitting device over the substrate, the substrate surface was washed with water and baking was performed at 200° C. for 1 hour.

After that, the substrate was transferred into a vacuum evaporation apparatus where the internal pressure was reduced to approximately 1×10⁴ Pa, and was subjected to vacuum baking at 170° C. for 30 minutes in a heating chamber of the vacuum evaporation apparatus, and then the substrate was cooled down for approximately 30 minutes.

Then, the substrate was fixed to a holder provided in the vacuum evaporation apparatus such that the surface on which the first electrode 101 was formed faced downward. Over the first electrode 101, N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) above and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer 111 was formed.

Over the hole-injection layer 111, PCBBiF was deposited by evaporation to a thickness of 110 nm, whereby a first hole-transport layer was formed.

Then, over the first hole-transport layer, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (ii) above, 9-(2-naphthyl)-9′-phenyl-3,3′-bi-9H-carbazole (abbreviation: βNCCP) represented by Structural Formula (iii) above, and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) represented by Structural Formula (iv) above were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a first light-emitting layer was formed.

Next, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (v) above was deposited by evaporation to a thickness of 10 nm, whereby a first electron-transport layer was formed.

After the first electron-transport layer was formed, 2,2′-([2,2′-bipyridine]-6,6′-diyl)bis(4-phenylbenzo[h]quinazoline) (abbreviation: 6,6′(P-Bqn)2BPy) represented by Structural Formula (vi) above, 4-(3,4-diphenyl-1-pyrrolidinyl)-7-(2,3,3a,4,5,6,7,7a-octahydro-1H-isoindol-2-yl)-1,10-phenanthroline (abbreviation: Hid-DPPrdPhen) represented by Structural Formula (vii) above, and lithium oxide (Li₂O) were deposited by co-evaporation to a thickness of 5 nm such that the volume ratio of 6,6′(P-Bqn)2BPy to Hid-DPPrdPhen to Li₂O was 0.5:0.5:0.02, whereby a first layer was formed. Then, copper phthalocyanine (abbreviation: CuPc) represented by Structural Formula (viii) above was deposited by evaporation to a thickness of 2 nm, whereby a third layer was formed. Furthermore, PCBBiF and OCHD-003 were deposited by co-evaporation to a thickness of 10 nm such that the weight ratio of PCBBiF to OCHD-003 was 1:0.15, whereby a second layer was formed. Thus, an intermediate layer was formed.

Over the intermediate layer, PCBBiF was deposited by evaporation to a thickness of 50 nm, whereby a second hole-transport layer was formed.

Over the second hole-transport layer, 8mpTP-4mDBtPBfpm, βNCCP, and Ir(5mppy-d3)₂(mbfpypy-d₃) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a second light-emitting layer was formed.

Then, 2mPCCzPDBq was deposited by evaporation to a thickness of 20 nm, and 2,2′-(1,3-phenylene)bis(9-phenyl-1,10-phenanthroline) (abbreviation: mPPhen2P) represented by Structural Formula (ix) above was further deposited by evaporation to a thickness of 20 nm, whereby a second electron-transport layer was formed.

Then, tris(8-quinolinolato)aluminum (abbreviation: Alq₃) was deposited by evaporation to a thickness of 10 nm, whereby a first protective layer was formed.

Then, the substrate provided with the components up to the first protective layer was taken out from the vacuum evaporation apparatus and exposed to the air, and then aluminum oxide was deposited to a thickness of 30 nm by an ALD method using trimethylaluminum (abbreviation: TMA) as a precursor and water vapor as an oxidizer, whereby an aluminum oxide film was formed as a second protective layer.

Over the second protective layer, molybdenum was deposited to a thickness of 50 nm by a sputtering method, whereby a third protective layer was formed.

A photoresist was applied over the third protective layer, subjected to light exposure and development, and processed so as to correspond to the plurality of first electrodes and be independent of each other for each electrode at a resolution of 508 ppi.

The third protective layer was processed using the photoresist as a mask and using an etching gas containing SF₆ and oxygen (O₂), and the second protective layer was processed using the processed third protective layer as a hard mask and using an etching gas containing fluoroform (CHF₃) and helium (H₂). Then, the hole-injection layer, the first hole-transport layer, the first light-emitting layer, the first electron-transport layer, the intermediate layer, the second hole-transport layer, the second light-emitting layer, and the second electron-transport layer were processed using an etching gas containing oxygen (O₂).

After the organic compound layers were processed, the third protective layer was removed using an etching gas containing SF₆ and oxygen (O₂), whereas the second protective layer was left. Then, aluminum oxide was deposited to a thickness of 15 nm by an ALD method, whereby a fourth protective layer was formed.

Next, a layer of a photosensitive high molecular material was formed over the fourth protective layer to overlap with the first electrodes by a photolithography method. After heating was performed at 100° C. in an air atmosphere for 10 minutes, unnecessary portions of the first, second, and fourth protective layers were removed using a mixed acid aqueous solution containing hydrofluoric acid (HF), whereby the second electron-transport layer was exposed. At this time, the layer of the photosensitive high molecular material functions as a resist.

The substrate over which the second electron-transport layer was exposed was transferred into a vacuum evaporation apparatus where the internal pressure was reduced to approximately 1×10⁻⁴ Pa, and was subjected to vacuum baking at 100° C. for 60 minutes in a heating chamber of the vacuum evaporation apparatus.

After that, lithium fluoride (LiF) and ytterbium (Yb) were deposited by co-evaporation to a thickness of 1.5 nm such that the volume ratio of LiF to Yb was 1:0.5, whereby an electron-injection layer was formed. Then, silver (Ag) and magnesium (Mg) were deposited by co-evaporation to a thickness of 15 nm such that the volume ratio of Ag to Mg was 1:0.1, whereby the second electrode 102 was formed. Over the second electrode 102, a film of 4,4′,4″-(benzene-1,3,5-triyl)tri(dibenzothiophene) (abbreviation: DBT3P-II) represented by Structural Formula (x) above was formed to a thickness of 70 nm as a cap layer to improve light extraction efficiency.

Then, the light-emitting device was sealed using a glass substrate in a glove box containing a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. In this manner, the light-emitting device 2-1 was fabricated.

### (Method for Fabricating Light-Emitting Device 2-2)

A light-emitting device 2-2 was fabricated in a manner similar to that of the light-emitting device 2-1 except that Hid-DPPrdPhen used in the first layer of the light-emitting device 2-1 was replaced with 4-(2,3,3a,4,5,6,7,7a-octahydro-1H-isoindol-2-yl)-7-[3-(1-naphthyl)-1-pyrrolidinyl]-1,10-phenanthroline (abbreviation: Hid-αNPrdPhen) represented by Structural Formula (xi) above.

### (Method for Fabricating Comparative Light-Emitting Device 2)

A comparative light-emitting device 2 was fabricated in a manner similar to that of the light-emitting device 2-1 except that Hid-DPPrdPhen used in the first layer of the light-emitting device 2-1 was replaced with 4,7-di(2,3,3a,4,5,6,7,7a-octahydro-1H-isoindol-2-yl)-1,10-phenanthroline (abbreviation: Hid2Phen) represented by Structural Formula (xii) above.

Device structures of the light-emitting devices 2-1 and 2-2 and the comparative light-emitting device 2 are shown below.

TABLE 4

| | Light-emitting | | Comparative |
|---|---|---|---|
| Thickness | device | | light-emitting |
| (nm) | 2-1 | 2-2 | device 2 |

| Cap layer | 70 | DBT3P-II |
|---|---|---|
| Second electrode | 15 | Ag:Mg (1:0.1) |
| | 1.5 | LiF:Yb (1:0.5) |

Processing by photolithography

| Second-electron | 2 | 20 | mPPhen2P | | |
|---|---|---|---|---|---|
| transport layer | 1 | 20 | 2mPCCzPDBq | | |
| Second light-emitting layer | | 40 | 8mpTP-4mDBtPBfpm:βNCCP:Ir(5mppy-d₃)₂(mbfpypy-d₃) | | |
| | | | (0.5:0.5:0.1) | | |
| Second hole-transport layer | | 50 | PCBBiF | | |
| Intermediate | Second | 10 | PCBBiF:OCHD-003 | | |
| layer | layer | | (1:0.15) | | |
| | Third layer | 2 | CuPc | | |
| | First layer | 5 | 6,6′(P-Bqn)2BPy:*2:Li₂O | | |
| | | | (0.5:0.5:0.02) | | |
| First electron-transport layer | | 10 | 2mPCCzPDBq | | |
| First light-emitting layer | | 40 | 8mpTP-4mDBtPBfpm:βNCCP:Ir(5mppy-d₃)₂(mbfpypy-d₃) | | |
| | | | (0.5:0.5:0.1) | | |
| First hole-transport layer | | 110 | PCBBiF | | |
| Hole-injection layer | | 10 | PCBBiF:OCHD-003 | | |
| | | | (1:0.03) | | |
| First electrode | | 50 | ITSO | | |
| | | 100 | APC | | |
| Z | Z | Z | Z | Z | Z |

TABLE 5

| | *2 |
|---|---|
| Light-emitting device 2-1 | Hid-DPPrdPhen |
| Light-emitting device 2-2 | Hid-αNPrdPhen |
| Comparative light-emitting | Hid2Phen |
| device 2 | |

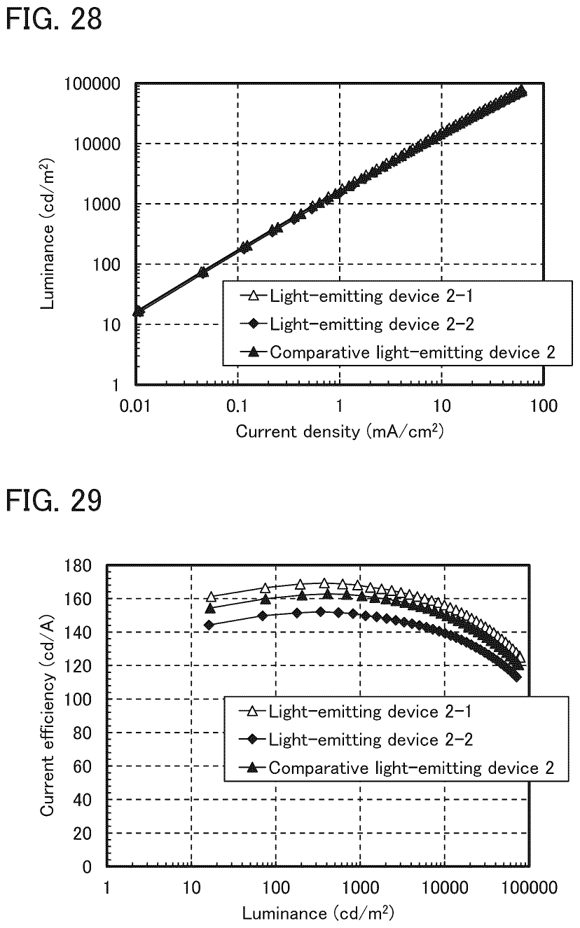

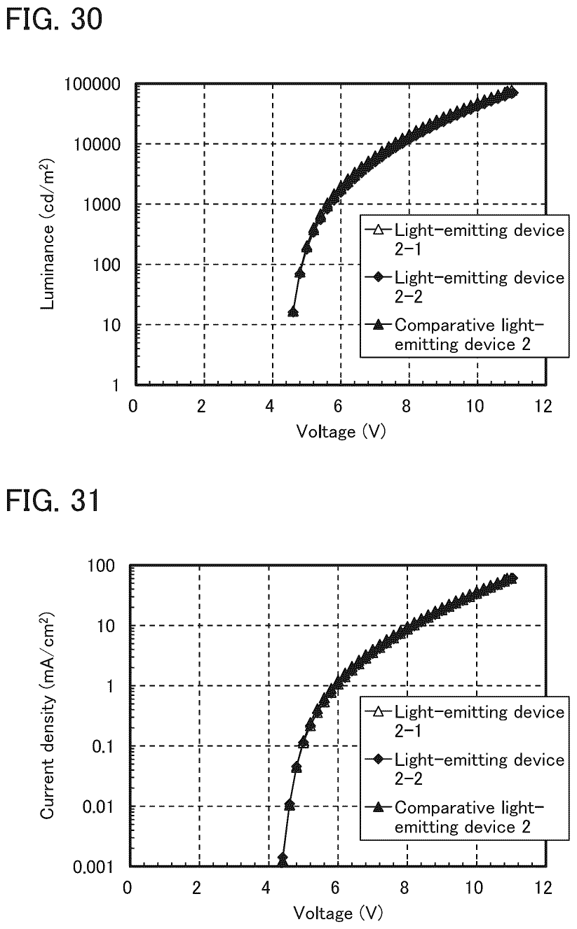

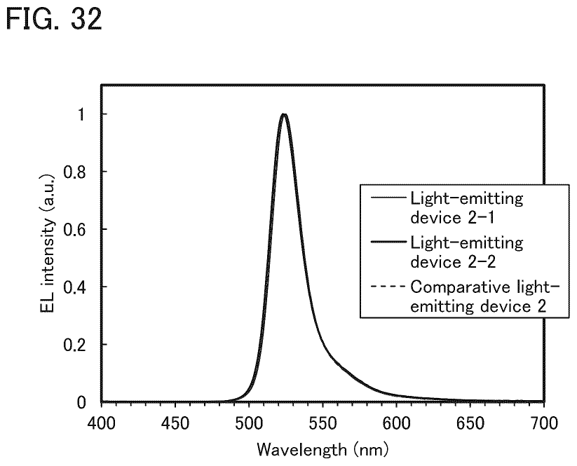

FIG. 28 shows luminance-current density characteristics of the light-emitting devices 2-1 and 2-2 and the comparative light-emitting device 2. FIG. 29 shows current efficiency-luminance characteristics thereof. FIG. 30 shows luminance-voltage characteristics thereof. FIG. 31 shows current density-voltage characteristics thereof. FIG. 32 shows electroluminescence spectra thereof. Table 6 shows the main characteristics of the light-emitting devices 2-1 and 2-2 and the comparative light-emitting device 2 at a luminance of approximately 1000 cd/m². The luminance, CIE chromaticity, and electroluminescence spectra were measured at normal temperature with a spectroradiometer SR-UL1R (TOPCON TECHNOHOUSE).

TABLE 6

| | | | Current | | | Current |
|---|---|---|---|---|---|---|
| | Voltage | Current | density | Chromaticity | Chromaticity | efficiency |
| | (V) | (mA) | (mA/cm²) | x | y | (cd/A) |
| Light-emitting device 2-1 | 6.20 | 0.0229 | 0.584 | 0.211 | 0.737 | 165 |
| Light-emitting device 2-2 | 6.40 | 0.0286 | 0.730 | 0.211 | 0.733 | 147 |
| Comparative light-emitting device 2 | 6.20 | 0.0266 | 0.679 | 0.209 | 0.736 | 159 |

FIGS. 28 to 32 and Table 6 reveal that each light-emitting device has high current efficiency and functions as a tandem light-emitting device even though the device was subjected to processing by a photolithography method involving exposure to the air in the process of fabricating the light-emitting device.

The above results reveal that a light-emitting device in which the organic compound represented by General Formula (G1) in Embodiment 1 is used for a first layer in a tandem light-emitting device exhibits favorable characteristics even after processing by a photolithography method.

### Example 13

### Synthesis Example 11

Described in this example is a method for synthesizing 4-(4-azatricyclo[5.2.2.0,2,6]undecan-4-yl)-7-(2,3,3a,4,5,6,7,7a-octahydro-1H-isoindol-2-yl)-1,10-phenanthroline (abbreviation: Hid-AcuPhen) represented by Structural Formula (122) in Embodiment 1. The structural of Hid-AcuPhen is shown below.

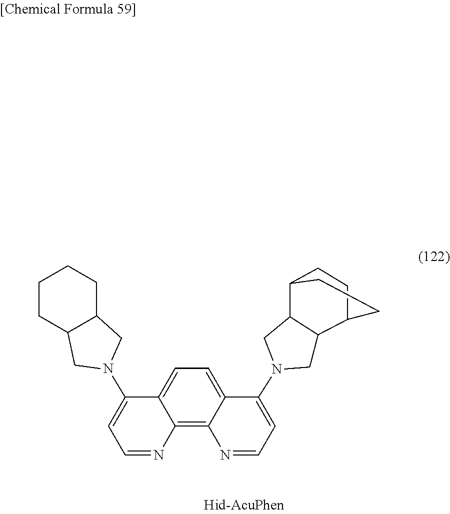 [SMILES: c1cc(N2CC3CCCCC3C2)c2ccc3c(N4CC5C6CCC(CC6)C5C4)ccnc3c2n1]

Into a 50-mL three-neck flask were put 1.00 g (2.96 mmol) of 4-chloro-7-(2,3,3a,4,5,6,7,7a-octahydro-1H-isoindol-2-yl)-1,10-phenanthroline, 0.69 g (3.68 mmol) of 4-azatricyclo[5.2.2.0,2,6]undecane hydrochloride, and 2.00 g (13.1 mmol) of 1,8-diazabicyclo[5.4.0]-7-undecene (DBU). The mixture was stirred at 100° C. under a nitrogen stream for 32 hours. After the stirring, the mixture was cooled down to room temperature. After water was added to the mixture, the mixture was subjected to extraction with dichloromethane. The extracted solution was concentrated to give a target crude product (1.07 g). The synthesis scheme of this synthesis example is shown in Formula (s11-1) below.

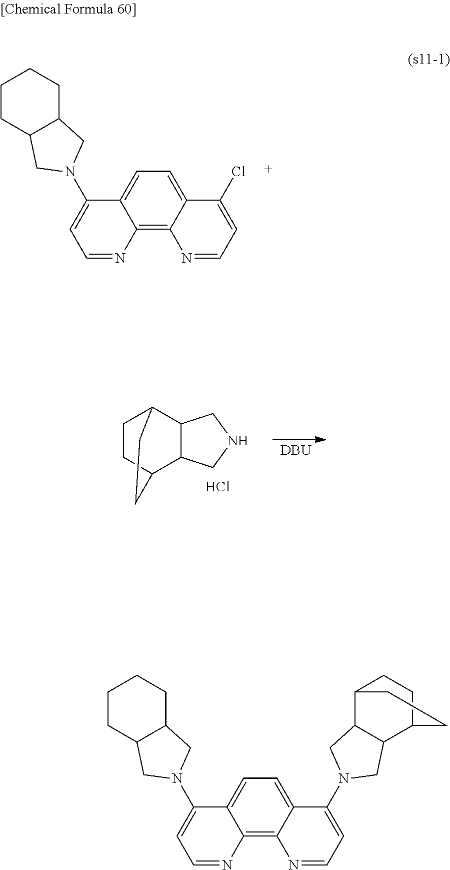

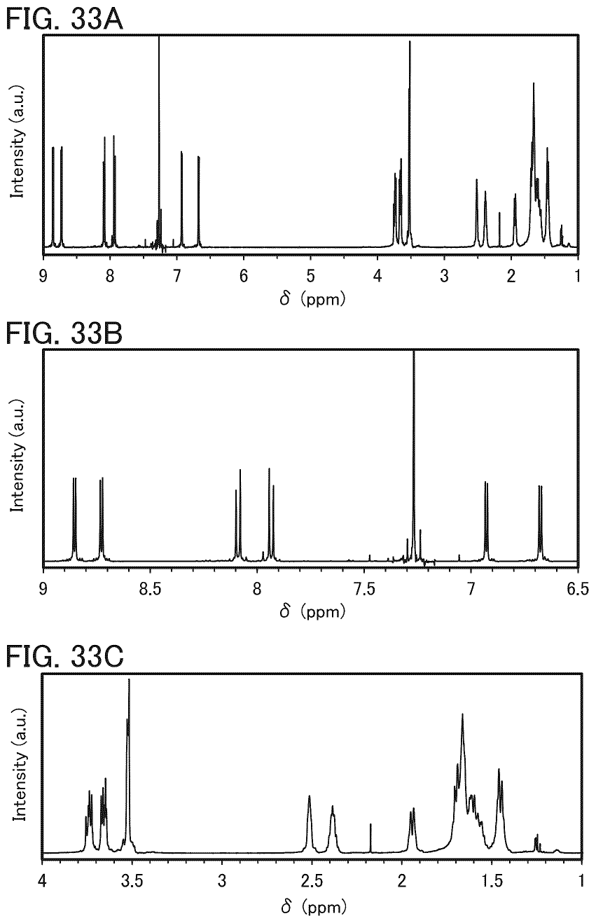

FIGS. 33A to 33C show the ¹H NMR spectrum of Hid-AcuPhen synthesized. FIG. 33B is an enlarged graph of FIG. 33A in the range from 6.5 ppm to 9.0 ppm, and FIG. 33C is an enlarged graph of FIG. 33A in the range from 1.0 ppm to 4.0 ppm. Results of ¹H NMR measurement are shown below. The results reveal that Hid-AcuPhen was obtained.

¹H NMR (500 MHz, CHLOROFORM-D): 6=8.85 (d, J=5.3 Hz, 1H), 8.72 (d, J=5.4 Hz, 1H), 8.08 (d, J=9.6 Hz, 1H), 7.93 (d, J=9.7 Hz, 1H), 6.92 (d, J=5.3 Hz, 1H), 6.67 (d, J=5.4 Hz, 1H), 3.75-3.64 (m, 4H), 3.54-3.48 (m, 4H), 2.54-2.48 (m, 2H), 2.41-2.35 (m, 2H), 1.99-1.89 (m, 2H), 1.74-1.53 (m, 12H), 1.49-1.39 (m, 4H).

### Example 14

### Synthesis Example 12

Described in this example is a method for synthesizing 4-(4-azatricyclo[5.2.1.0,2,6]decan-4-yl)-7-(2,3,3a,4,5,6,7,7a-octahydro-1H-isoindol-2-yl)-1,10-phenanthroline (abbreviation: Hid-AccPhen), which is the organic compound of one embodiment of the present invention. The structure of Hid-AccPhen is shown below.

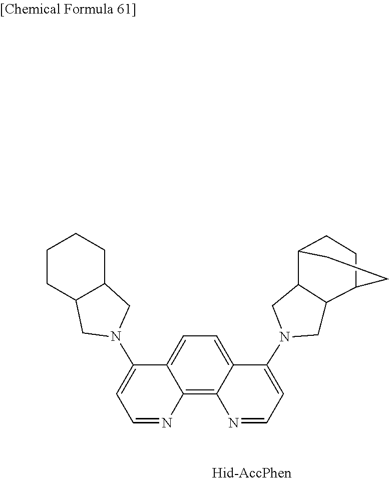 [SMILES: c1cc(N2CC3CCCCC3C2)c2ccc3c(N4CC5C6CCC(CC6)C5C4)ccnc3c2n1]

Into a 50-mL three-neck flask were put 1.0 g (3.0 mmol) of 4-chloro-7-(2,3,3a,4,5,6,7,7a-octahydro-1H-isoindol-2-yl)-1,10-phenanthroline, 0.64 g (3.7 mmol) of 4-azatricyclo[5.2.1.0,2,6]decane hydrochloride, and 1.1 g (7.2 mmol) of 1,8-diazabicyclo[5.4.0]-7-undecene (abbreviation: DBU). The mixture was stirred at 100° C. under a nitrogen stream for 8 hours. After the stirring, the mixture was cooled down to room temperature. After water was added to the mixture, the mixture was subjected to extraction with dichloromethane. The extracted solution was concentrated to give a target substance. The synthesis scheme of Hid-AccPhen is shown in Formula (s12-1) below.

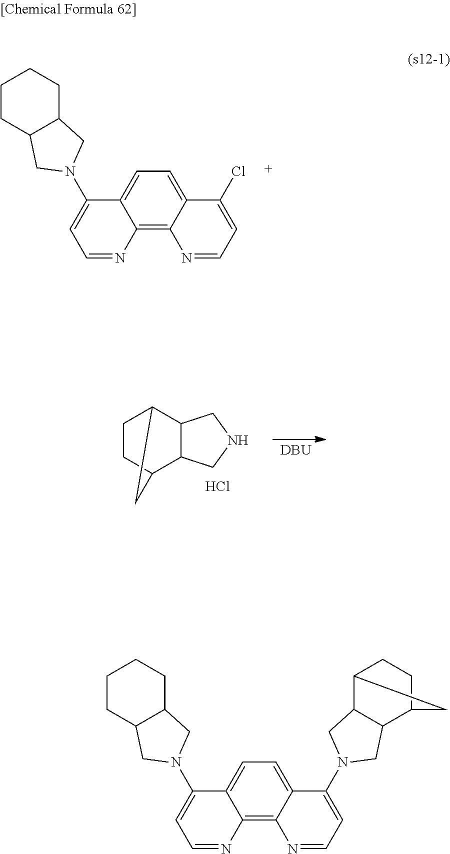

The target substance obtained was subjected to mass measurement using gas chromatography/mass spectrometry (GC/MS). As a result, a signal was observed at m/z 438 while the mass of the target substance was calculated to be 438, revealing that 4-(4-azatricyclo[5.2.1.0,2,6]decan-4-yl)-7-(2,3,3a,4,5,6,7,7a-octahydro-1H-isoindol-2-yl)-1,10-phenanthroline was obtained.

In the GC/MS analysis, mass spectrometry (MS) analysis was carried out by a direct exposure probe (DEP) method using DEP-ISQ 7610 (Thermo Fisher Scientific K.K.). The ion source temperature was set to 200° C. The initial probe current was set to 0 mA and retained for 30 seconds. Then, the probe current was increased at a rate of 20 mA/s for 30 seconds. After that, the probe current was increased to 800 mA and retained for 30 seconds.

This application is based on Japanese Patent Application Serial No. 2024-134263 filed with Japan Patent Office on Aug. 9, 2024, the entire contents of which are hereby incorporated by reference.

What is claimed is:

1. An organic compound represented by General Formula (G1) or (G2): 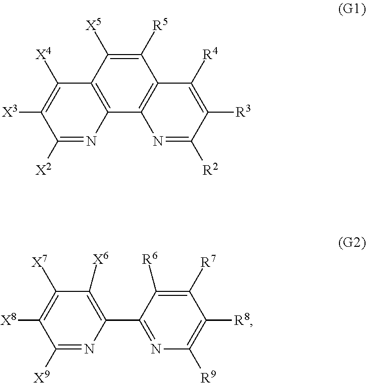
wherein any one of X² to X⁵ or any one of X⁶ to X⁹ represents a halogen or a trifluoromethanesulfonyl group and the others represent hydrogens,
wherein any one of R² to R⁵ or any one of R⁶ to R⁹ represents an aliphatic cyclic amino group represented by General Formula (g1) and the others represent hydrogens,
wherein a carbon to which the halogen or the trifluoromethanesulfonyl group is bonded and a carbon to which the aliphatic cyclic amino group represented by General Formula (g1) is bonded are at line-symmetrical positions in a 1,10-phenanthroline skeleton or a 2,2′-bipyridine skeleton which is a main skeleton in General Formula (G1) or (G2), 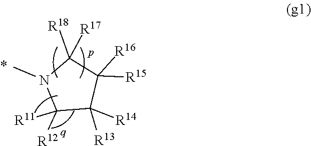
wherein R¹¹ to R¹⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3, and
wherein any two of R¹¹ to R¹⁸ are bonded to each other to form a ring or not bonded to each other.

2. The organic compound according to claim 1, wherein the organic compound is represented by any one of General Formulae (G1-1) to (G1-4): 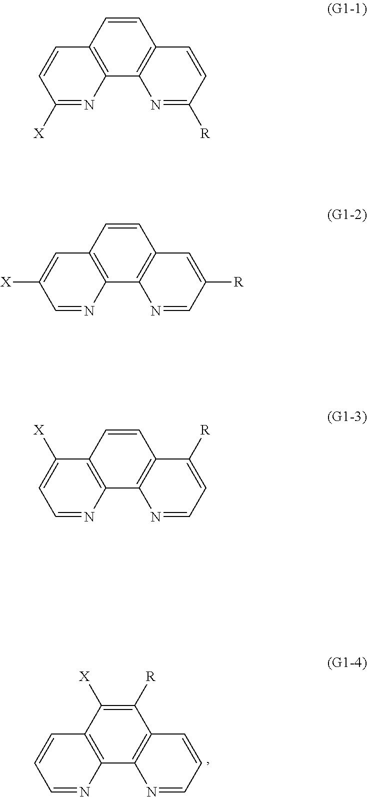
wherein X represents a halogen or a trifluoromethanesulfonyl group,
wherein R represents an aliphatic cyclic amino group represented by General Formula 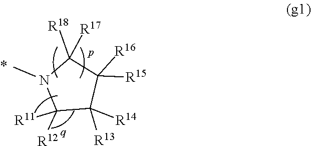
wherein R¹¹ to R¹⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3, and
wherein any two of R¹¹ to R¹⁸ are bonded to each other to form a ring or not bonded to each other.

3. The organic compound according to claim 1, wherein the organic compound is represented by any one of General Formulae (G2-1) to (G2-4): 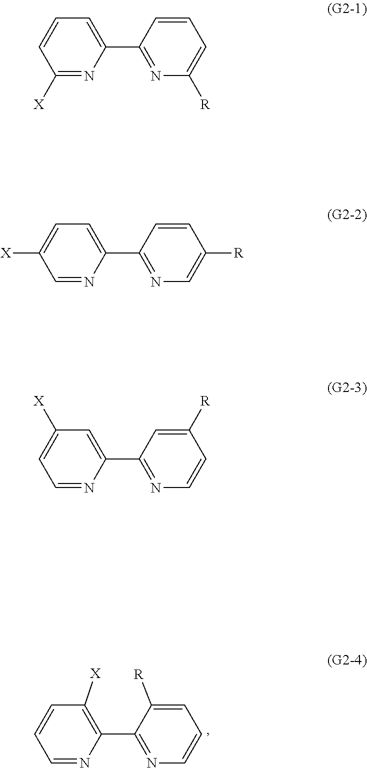
wherein X represents a halogen or a trifluoromethanesulfonyl group,
wherein R represents an aliphatic cyclic amino group represented by General Formula (g1), 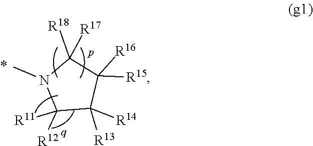
wherein R¹¹ to R¹⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3, and
wherein any two of R¹¹ to R¹⁸ are bonded to each other to form a ring or not bonded to each other.

4. An organic compound represented by General Formula (G3): 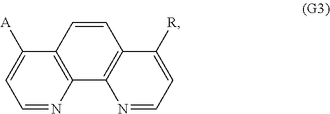
wherein R represents a group by Formula (g1),
wherein A represents a group represented by General Formula (g2) or (g3),
wherein A and R are different substituents, 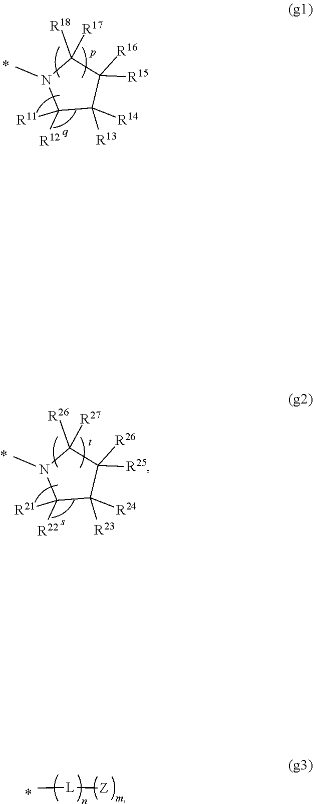
wherein R¹¹ to R¹⁸ and R²¹ to R²⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p, q, s, and t each independently represent 0 to 3,
wherein any two of R¹¹ to R¹⁸ are bonded to each other to form a ring or not bonded to each other,
wherein any two of R²¹ to R²⁸ are bonded to each other to form a ring or not bonded to each other,
wherein Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, a substituted or unsubstituted secondary amino group having 2 to 10 carbon atoms, a cyano group, a halogen, a hydroxy group, an amide group, and a carbonyl group,
wherein m represents an integer of 1 to 3,
wherein L represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and
wherein n represents an integer of 0 to 3.

5. The organic compound according to claim 4, wherein the organic compound is represented by General Formula (G3-1): 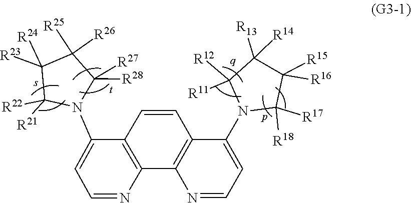
wherein R¹¹ to R¹⁸ and R²¹ to R²⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p, q, s, and t each independently represent 0 to 3,
wherein any two of R¹¹ to R¹⁸ are bonded to each other to form a ring or not bonded to each other,
wherein any two of R²¹ to R²⁸ are bonded to each other to form a ring or not bonded to each other, and
wherein different substituents are bonded to the 4- and 7-positions in a 1,10-phenanthroline skeleton in General Formula (G3-1).

6. The organic compound according to claim 4, wherein the organic compound is represented by General Formula (G3-2): 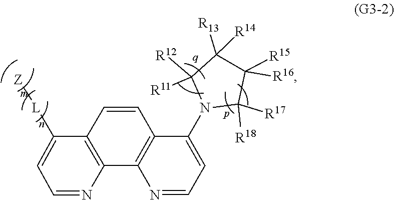
wherein R¹¹ to R¹⁸ each independently represent any one of a hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3,
wherein any two of R¹¹ to R¹⁸ are bonded to each other to form a ring or not bonded to each other,
wherein Z represents any one of a substituted or unsubstituted alkyl group having 1 to 10 carbon atoms, a substituted or unsubstituted cycloalkyl group having 3 to 10 carbon atoms, a substituted or unsubstituted monovalent aromatic hydrocarbon group having 6 to 30 carbon atoms, a substituted or unsubstituted monovalent heteroaromatic ring group having 1 to 30 carbon atoms, a substituted or unsubstituted alkoxy group having 1 to 10 carbon atoms, a substituted or unsubstituted secondary amino group having 2 to 10 carbon atoms, a cyano group, a halogen, a hydroxy group, an amide group, and a carbonyl group,
wherein m represents an integer of 1 to 3,
wherein L represents any one of a substituted or unsubstituted alkylene group having 1 to 10 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and
wherein n represents an integer of 0 to 3.

7. The organic compound according to claim 4, wherein an aliphatic cyclic amino group represented by General Formula (g2) is condensed with an aromatic ring having 6 to 10 carbon atoms.

8. The organic compound according to claim 5, wherein one aliphatic cyclic amino group in General Formula (G3-1) is condensed with an aromatic ring having 6 to 10 carbon atoms.

9. A method for synthesizing a 1,10-phenanthroline derivative or a 2,2′-bipyridine derivative having two different substituents bonded to respective carbons at symmetrical positions, the method comprising:
reacting, using an inorganic base and a solvent, an aliphatic cyclic amine with a 1,10-phenanthroline derivative or a 2,2′-bipyridine derivative having two halogens or trifluoromethanesulfonyl groups bonded to respective carbons at symmetrical positions.

10. The method for synthesizing according to claim 9, wherein the reacting is performed by heating.

11. The method for synthesizing according to claim 9, wherein the inorganic base is potassium carbonate or potassium acetate.

12. The method for synthesizing according to claim 9,
wherein after the reacting, a 1,10-phenanthroline derivative or a 2,2′-bipyridine derivative having the aliphatic cyclic amine bonded to one of the carbons at the symmetrical positions is obtained,
wherein the inorganic base is potassium carbonate or potassium acetate, and
wherein the method further comprises introducing another substituent to the other of the carbons at the symmetrical positions.

13. The method for synthesizing according to claim 9, wherein N-methyl-2-pyrrolidone is used as the solvent.
